SEQUENCE LISTING (12 sequences)

SEQ ID NO: 1 - protein, 221 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 2 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 3 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 4 - protein, 216 aa, synthetic construct
QSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 5 - protein, 445 aa, synthetic construct
EVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 219 aa, synthetic construct
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 444 aa, synthetic construct
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 214 aa, synthetic construct
DIQMTQSPSSLSASVGDRVTITCKASDHINNWLAWYQQKPGQAPRLLISGATSLETGVPSRFSGSGTGKDYTLTISSLQPEDFATYYCQQYWSTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 444 aa, synthetic construct
QVQLVQSGAELKKPGASVKLSCKASGYTFTSYGISWVKQATGQGLEWIGEIYPRSGNTYYNEKFKGRATLTADKSTSTAYMELRSLRSEDSAVYFCARSTTVRPPGIWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 214 aa, synthetic construct
SYVLTQSPSVSVAPGQTARITCGGNNIGSKSVHWYQQKPGQAPVLVVYDDSDRPSGIPERFSASNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 450 aa, synthetic construct
QLLLQESGPGLVKPSETLSLTCTVSGGSLSSSFSYWVWIRQPPGKGLEWIGTIYYSGNTYYNPSLKSRLTISVDTSKNHFSLKLSSVTAADTAVYYCARRAGILTGYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 15 aa, synthetic construct
EPKSCDKTHTCPPCP

(19) United States
(12) Patent Application Publication
(10) Pub. No.: US 2025/0084171 A1
(43) Pub. Date: Mar. 13, 2025

(54) METHODS FOR TREATING MYOSITIS USING FCRN ANTAGONISTS

(71) Applicant: argenx BV, Ghent (BE)

(72) Inventors: Sebastian Paul van der Woning, Ghent (BE); Olivier Boyer, Mont Saint Aignan (FR); Laurent Drouot, Mont Saint Aignan (FR)

(21) Appl. No.: 18/810,851

(22) Filed: Aug. 21, 2024

Related U.S. Application Data
(63) Continuation of application No. PCT/EP2023/054065, filed on Feb. 17, 2023.
(60) Provisional application No. 63/407,369, filed on Sep. 16, 2022.
(60) Provisional application No. 63/365,824, filed on Jun. 3, 2022.
(60) Provisional application No. 63/269,430, filed on Mar. 16, 2022.
(60) Provisional application No. 63/268,312, filed on Feb. 21, 2022.

(51) Int. Cl. C07K 16/28; A61K 39/00; A61K 45/06; A61P 37/06; G01N 33/68

(52) U.S. Cl. CPC C07K 16/283; A61K 45/06; A61P 37/06; G01N 33/6854; A61K 2039/505; A61K 2039/545; C07K 2317/52; G01N 2800/52

(57) ABSTRACT

Provided herein are methods of treating myositis using an effective amount of a human neonatal Fc receptor (FcRn) antagonist. FcRn antagonists for use in the treatment of myositis and for use in the manufacture of a medicament for the treatment of myositis are also provided herein.

### CROSS REFERENCE TO RELATED APPLICATIONS

This application is a Continuation of PCT International Application No. PCT/EP2023/054065, filed on Feb. 17, 2023, which claims the benefit of and priority to U.S. Provisional Application No. 63/407,369 filed on Sep. 16, 2022, U.S. Provisional Application No. 63/365,824 filed on Jun. 3, 2022, U.S. Provisional Application No. 63/269,430 filed on Mar. 16, 2022, and U.S. Provisional Application No. 63/268,312 filed on Feb. 21, 2022, the entire contents of each of which are hereby incorporated by reference in their entirety. SUBMISSION OF SEQUENCE LISTING XML

The content of the following submission on Sequence Listing XML is incorporated herein by reference in its entirety: a computer readable form (CRF) of the Sequence Listing (file name: P234756W000—SEQ LISTING. xml, date created Feb. 2, 2023, size 14544 bytes).

### FIELD

The present disclosure relates to methods of treating myositis, including but not limited to immune-mediated necrotizing myopathy (IMNM), dermatomyositis (DM), polymyositis (PM), and antisynthetase syndrome (ASyS). The methods involve use of an antagonist of human neonatal Fc receptor (FcRn), which in certain embodiments is efgartigimod.

### BACKGROUND

It is estimated that more than 2.5% of the human population is affected by autoantibody-driven autoimmune diseases, in which autoreactive antibodies are directly pathogenic. Idiopathic inflammatory myopathies (IIMs) are a heterogeneous group of diseases that includes subtypes with varying pathologies primarily targeting muscle and/or skin and other organs. In many subsets, there is a potential role of myositis-specific autoantibodies, most of which are immunoglobulin G (IgG) in the disease pathogenesis.

Many patients with IIM have persistent impairment of muscle function, which leads to difficulties in daily life activities and a low health-related quality of life. The typical treatment for IIM is high-dose glucocorticoids combined with immunosuppressive drugs. The deleterious long-term effects of corticosteroids have been well established and include osteoporosis, cataracts, and weight gain. There are no therapies approved by the United States Food and Drug Administration (FDA) or the European regulatory authorities based on results of randomized controlled trials for IMNM and PM. Only 1 licensed treatment (10% intravenous immunoglobulin [IVIg]) is available for adults with DM that was approved based on results of randomized controlled clinical trials.

Accordingly, there is a need in the art for improved IIM treatment options that permit tapering or elimination of corticosteroid treatment.

Therapeutic antagonism of the neonatal Fc receptor (FcRn), a major histocompatibility complex class I-like molecule that is involved in the recycling of immunoglobulin G (IgG) and is thus responsible for the long half-life of IgG, has been explored as a strategy to treat IgG-mediated autoimmune diseases such as generalized myasthenia gravis (gMG), immune thrombocytopenia (ITP), and pemphigus (pemphigus vulgaris (PV) and pemphigus foliaceus (PF)). The remarkable clinical efficacy of FcRn antagonism appears to be directly linked to early removal of pathogenic IgG autoantibodies from circulation.

Given their ability to reduce IgG levels, FcRn antagonists may provide a safer, more effective treatment option for patients with IIM.

### SUMMARY

The instant disclosure demonstrates that FcRn antagonists are highly effective in treating myositis. Accordingly, the instant disclosure is broadly directed to methods for treating myositis with FcRn antagonists.

In an aspect, provided herein is a method for treating myositis in a subject in need thereof, the method comprising administering to the subject an effective amount of a human neonatal Fc receptor (FcRn) antagonist.

In some embodiments, the FcRn antagonist comprises two, three, or four FcRn binding regions.

In some embodiments, the FcRn antagonist comprises or consists of a variant Fc region or FcRn binding fragment thereof. In some embodiments, the variant Fc region or FcRn binding fragment thereof binds to FcRn with a higher affinity at pH 5.5 as compared to a corresponding wild-type Fc region. In some embodiments, the variant Fc region or FcRn binding fragment thereof binds to FcRn with a higher affinity at pH 6.0 as compared to a corresponding wild-type Fc region. In some embodiments, the variant Fc region or FcRn binding fragment thereof binds to FcRn with a higher affinity at pH 7.4 as compared to a corresponding wild-type Fc region.

In some embodiments, the variant Fc region comprises or consists of two Fc domains which form a homodimer or heterodimer. In some embodiments, the variant Fc region comprises or consists of a first Fc domain and a second Fc domain which form a homodimer or heterodimer.

In some embodiments, the FcRn antagonist comprises an Fc region comprising amino acids Y, T, E, K, F, and Y at EU positions 252, 254, 256, 433, 434, and 436, respectively. In some embodiments, the first Fc domain and/or the second Fc domain comprise amino acids Y, T, E, K, and F at EU positions 252, 254, 256, 433, and 434, respectively. In some embodiments, the first Fc domain and/or the second Fc domain comprise amino acids Y, T, E, K, F, and Y at EU positions 252, 254, 256, 433, 434, and 436, respectively.

In some embodiments, the amino acid sequence of at least one of the Fc domains is selected from the group consisting of SEQ ID NO: 1, SEQ ID NO: 2, and SEQ ID NO: 3. In some embodiments, the amino acid sequence of both of the Fc domains is independently selected from the group consisting of SEQ ID NO: 1, SEQ ID NO: 2, and SEQ ID NO: 3.

In some embodiments, the first Fc domain and/or the second Fc domain comprise an amino acid sequence independently selected from the group consisting of SEQ ID NO: 1, SEQ ID NO: 2, and SEQ ID NO: 3. In some embodiments, the first Fc domain and the second Fc domain comprise an amino acid sequence independently selected from the group consisting of SEQ ID NO: 1, SEQ ID NO: 2, and SEQ ID NO: 3. In some embodiments, the FcRn antagonist is efgartigimod.

In some embodiments, the FcRn antagonist is an anti-FcRn antibody. In some embodiments, the anti-FcRn antibody is selected from the group consisting of rozanolixizumab, nipocalimab, orilanolimab, and batoclimab.

In some embodiments, the FcRn antagonist is administered to the subject at a fixed dose of about 20 mg to about 20,000 mg or at a dose of about 0.2 mg/kg to about 200 mg/kg. In some embodiments, the FcRn is administered to the subject at a fixed dose of about 200 mg to about 20,000 mg or at a dose of about 2 mg/kg to about 200 mg/kg.

In some embodiments, the FcRn antagonist is administered to the subject at a fixed dose of 20 mg to 20,000 mg or at a dose of 0.2 mg/kg to 200 mg/kg. In some embodiments, the FcRn is administered to the subject at a fixed dose of 200 mg to 20,000 mg or at a dose of 2 mg/kg to 200 mg/kg.

In some embodiments, the FcRn antagonist is administered subcutaneously once weekly, once every two weeks, once every three weeks, once every four weeks, once monthly, or once every six weeks. In some embodiments, the FcRn antagonist is administered subcutaneously at a fixed dose of about 20 mg to about 20,000 mg once weekly, once every two weeks, once every three weeks, once every four weeks, once monthly, or once every six weeks. In some embodiments, the FcRn antagonist is administered subcutaneously at a fixed dose of about 200 mg to about 20,000 mg once weekly, once every two weeks, once every three weeks, once every four weeks, once monthly or once every six weeks. In some embodiments, the FcRn antagonist is administered subcutaneously at a fixed dose of about 1000 mg or about 2000 mg once weekly, once every two weeks, once every three weeks, once every four weeks, once monthly or once every six weeks. In some embodiments, the FcRn antagonist is first administered subcutaneously at a fixed dose of about 1000 mg twice on the same day.

In some embodiments, the FcRn antagonist is administered subcutaneously at a fixed dose of 20 mg to 20,000 mg once weekly, once every two weeks, once every three weeks, once every four weeks, once monthly, or once every six weeks. In some embodiments, the FcRn antagonist is administered subcutaneously at a fixed dose of 200 mg to 20,000 mg once weekly, once every two weeks, once every three weeks, once every four weeks, once monthly or once every six weeks. In some embodiments, the FcRn antagonist is administered subcutaneously at a fixed dose of 750 mg to 3000 mg once weekly or every two weeks. In some embodiments, the FcRn antagonist is administered subcutaneously at a fixed dose of 1000 mg or 2000 mg once weekly, once every two weeks, once every three weeks, once every four weeks, once monthly or once every six weeks. In some embodiments, the FcRn antagonist is first administered subcutaneously at a fixed dose of 1000 mg twice on the same day.

In some embodiments, the FcRn antagonist is efgartigimod and the efgartigimod is administered subcutaneously at a fixed dose of about 800 to about 1200 mg once weekly. In some embodiments, the FcRn antagonist is efgartigimod and the efgartigimod is administered subcutaneously at a fixed dose of 800 to 1200 mg once weekly. In some embodiments, the efgartigimod is administered subcutaneously at a fixed dose of about 1000 mg once weekly. In some embodiments, the efgartigimod is administered subcutaneously at a fixed dose of 1000 mg once weekly.

In some embodiments, the FcRn antagonist is co-formulated with hyaluronidase and administered subcutaneously. In some embodiments, the hyaluronidase is recombinant human hyaluronidase PH20 (rHuPH20). In some embodiments, the rHuPH20 is administered at about 11,000 U once per week.

In some embodiments, the FcRn antagonist is administered intravenously once weekly or once every two weeks. In some embodiments, the FcRn antagonist is administered intravenously at a dose of from about 0.2 mg/kg to about 200 mg/kg once weekly or once every two weeks. In some embodiments, the FcRn antagonist is administered intravenously at a dose of from about 2 mg/kg to about 200 mg/kg once weekly or once every two weeks. In some embodiments, the FcRn antagonist is administered intravenously at a dose of about 3 mg/kg to about 60 mg/kg once weekly or once every two weeks. In some embodiments, the FcRn antagonist is administered intravenously at a dose of about 10 mg/kg once weekly or once every two weeks. In some embodiments, the FcRn antagonist is administered intravenously at a dose of about 25 mg/kg once weekly or once every two weeks.

In some embodiments, the FcRn antagonist is administered intravenously at a dose of from 0.2 mg/kg to 200 mg/kg once weekly or once every two weeks. In some embodiments, the FcRn antagonist is administered intravenously at a dose of from 2 mg/kg to 200 mg/kg once weekly or once every two weeks. In some embodiments, the FcRn antagonist is administered intravenously at a dose of 3 mg/kg to 60 mg/kg once weekly or once every two weeks. In some embodiments, the FcRn antagonist is administered intravenously at a dose of 10 mg/kg to 30 mg/kg once weekly or every two weeks. In some embodiments, the FcRn antagonist is administered intravenously at a dose of 10 mg/kg once weekly or once every two weeks. In some embodiments, the FcRn antagonist is administered intravenously at a dose of 25 mg/kg once weekly or once every two weeks.

In some embodiments, the FcRn antagonist is administered for 52 weeks or less. In some embodiments, the FcRn antagonist is administered for 24 weeks or less.

In some embodiments, the FcRn antagonist is administered for at least 24 weeks. In some embodiments, the FcRn antagonist is administered for at least 52 weeks.

In some embodiments, the method further comprises administering to the subject a dose of corticosteroid.

In some embodiments, the dose of corticosteroid is decreased after 16 weeks of treatment with the FcRn antagonist. In some embodiments the dose of corticosteroid is decreased after 24 weeks of treatment with the FcRn antagonist.

In some embodiments, the corticosteroid is prednisone. In some embodiments, the dose of prednisone is decreased after 16 weeks of treatment with the FcRn antagonist if the dose of prednisone is administered to the subject at more than 5 mg per day prior to the dose of prednisone being decreased. In some embodiments, following 16 weeks of treatment with the FcRn antagonist, the daily dose of prednisone is decreased by 2.5 mg and the decreased dose is maintained for four weeks. In some embodiments, following 16 weeks of treatment with the FcRn antagonist, the daily dose of prednisone is decreased by 2.5 mg every four weeks for one or more four-week periods. In some embodiments, the prednisone is administered at no more than 20 mg per day.

In some embodiments, the dose of prednisone is decreased after 24 weeks of treatment with the FcRn antagonist if the dose of prednisone is administered to the subject at more than 7.5 mg per day prior to the dose of prednisone being decreased. In some embodiments, following 24 weeks of treatment with the FcRn antagonist, the daily dose of prednisone is decreased by 2.5 mg and the decreased dose is maintained for four weeks. In some embodiments, following 24 weeks of treatment with the FcRn antagonist, the daily dose of prednisone is decreased by 2.5 mg every four weeks for one or more four-week periods.

In some embodiments, the myositis is selected from the group consisting of immune-mediated necrotizing myopathy (IMNM), dermatomyositis (DM), juvenile dermatomyositis (JDM), polymyositis (PM), and antisynthetase syndrome (ASyS).

In some embodiments, the subject is diagnosed with an idiopathic inflammatory myopathy (IIM). In some embodiments, the IIM is selected from the group consisting of IMNM, DM, JDM, PM, and ASyS.

In some embodiments, the myositis is IMNM.

In some embodiments, the subject has anti-signal recognition particle (SRP) autoantibodies. In some embodiments, the subject shows a reduction in serum anti-SRP autoantibody level following administration of the anti-FcRn antagonist. In some embodiments, the reduction in the serum anti-SRP autoantibody level following administration of the anti-FcRn antagonist is at least 10%, at least 25%, at least 50%, at least 75%, at least 80%, at least 90%, at least 95%, at least 96%, at least 97%, at least 98%, or at least 99% of the subject's serum anti-SRP autoantibody level prior to administration of the anti-FcRn antagonist. In some embodiments, anti-SRP autoantibodies are undetectable in the subject's serum after administration of the FcRn antagonist.

In some embodiments, the subject has anti-3-hydroxy-3-methylglutaryl coenzyme A reductase (HMGCR) autoantibodies. In some embodiments, the subject shows a reduction in serum anti-HMGCR autoantibody level following administration of the anti-FcRn antagonist. In some embodiments, the reduction in the serum anti-HMGCR autoantibody level following administration of the anti-FcRn antagonist is at least 10%, at least 25%, at least 50%, at least 75%, at least 80%, at least 90%, at least 95%, at least 96%, at least 97%, at least 98%, or at least 99% of the subject's serum anti-SRP autoantibody level prior to administration of the anti-FcRn antagonist. In some embodiments, anti-HMGCR autoantibodies are undetectable in the subject's serum after administration of the FcRn antagonist.

In some embodiments, the myositis is DM or JDM. In some embodiments, the subject has an active DM skin rash. In some embodiments, the active DM skin rash is selected from the group consisting of Gottron's papules, Gottron's signs, and heliotrope rash.

In some embodiments, the myositis is PM or ASyS. In some embodiments, the subject has a detectable serum level of a myositis-associated antibody (MAA) or a myositis-specific antibody (MSA). In some embodiments, the MSA is an anti-aminoacyl-tRNA synthetase antibody. In some embodiments, the anti-aminoacyl-tRNA synthetase antibody is selected from the group consisting of anti-Jo-1, anti-PL-7, anti-PL-12, anti-EJ, and anti-OJ antibodies. In some embodiments, the MSA is selected from the group consisting of anti-SRP, anti-HMGCR, anti-Mi-2, anti-TIF1, anti-SAE, anti-NXP, and anti-MDA5 antibodies. In some embodiments, the MAA is selected from the group consisting of anti-PM/Scl 75, anti-Ku, anti-snRNP, anti-Ro52 (SSA), anti-Ro/60 (SSA), and anti-La (SSB) antibodies. In some embodiments, the reduction in the serum level of MAA or MSA following administration of the anti-FcRn antagonist is at least 10%, at least 25%, at least 50%, at least 75%, at least 80%, at least 90%, at least 95%, at least 96%, at least 97%, at least 98%, or at least 99% of the subject's serum level of the MAA or MSA prior to administration of the anti-FcRn antagonist. In some embodiments, the serum level of the MAA or MSA are undetectable after administration of the FcRn antagonist.

In some embodiments, the subject shows at least a 20 point improvement in TIS. In some embodiments, the subject shows at least a minimal improvement in TIS within 12 weeks or less. In some embodiments, the subject shows at least a 20 point improvement in TIS within 6 weeks or less.

In some embodiments, the subject shows at least a 40 point improvement in TIS.

In some embodiments, the subject shows at least a 60 point improvement in TIS.

In some embodiments, the subject shows at least a 20% improvement in MMT8 score. In some embodiments, the subject shows at least a 30% improvement in MMT8 score.

In some embodiments, the subject shows at least a 20% decrease in patient's global assessment of disease activity (PGA). In some embodiments, the subject shows at least a 30% decrease in PGA.

In some embodiments, the method further comprises measuring at least one muscle-associated enzyme in the subject, wherein a reduction in at least one muscle-associated enzyme in the subject is indicative of disease improvement. In some embodiments, the at least one muscle-associated enzyme is selected from the group consisting of creatine kinase, alanine aminotransferase, aspartate aminotransaminase, lactate dehydrogenase, and aldolase. In some embodiments, the at least one muscle-associated enzyme is creatine kinase.

In an aspect, an FcRn antagonist for use in the treatment of myositis, wherein the treatment is performed according to any of the methods described herein, is also provided.

In an aspect, an FcRn antagonist for use in the manufacture of a medicament for the treatment of myositis, wherein the treatment is performed according to any of the methods described herein, is also provided.

In an aspect, provided herein is a method for monitoring efficacy of treatment of myositis in a subject following treatment with a first FcRn antagonist, the method comprising: a) measuring in vitro a serum level of a myositis-associated antibody (MAA) or a myositis-specific antibody (MSA) in a blood sample taken from the subject; and b) comparing the serum level of the MAA or the MSA to a reference value associated with myositis in the subject, wherein the treatment is not effective if the serum level of the MAA or the MSA in the sample is greater than or equal to the reference value, and wherein the treatment is effective if the serum level of the MAA or the MSA is less than the reference value.

In an aspect, provided herein is a method of treating myositis in a subject that has received a first FcRn antagonist and is receiving a corticosteroid dosing regimen, the method comprising: a) administering to the subject an effective amount of a second FcRn antagonist; b) measuring in vitro a serum level of an MAA or an MSA in a blood sample taken from the subject; and c) comparing the serum level of the MAA or the MSA to a reference value associated with myositis in the subject, wherein the corticosteroid dosing regimen is maintained if the serum level of the MAA or the MSA in the sample is greater than or equal to the reference value, or wherein the corticosteroid dosing regimen is tapered if the serum level of the MAA or the MSA is less than the reference value.

In an aspect, provided herein is a method for treating myositis in a subject comprising: (a) administering to the subject one or more initial doses of an effective amount of a first FcRn antagonist, (b) administering to the subject one or more further doses of an effective amount of a second FcRn antagonist if the serum level of an MAA or an MSA in the subject after step (a) is greater than or equal to a reference value associated with myositis in the subject, or discontinuing treatment with the first FcRn antagonist if the serum level of the MAA or the MSA in the subject after step (a) is less than a reference value associated with active disease in the subject.

In an aspect, provided herein is a method for treating myositis in a subject, the method comprising: administering to the subject an effective amount of a second FcRn antagonist, wherein the myositis has relapsed in the subject following prior therapy with a first FcRn antagonist and wherein the subject has a serum level of an MAA or an MSA that is greater than or equal to a reference value associated with myositis in the subject.

In an aspect, provided herein is a method for determining if a subject that has previously been treated for myositis using a first FcRn antagonist requires further treatment with a second FcRn antagonist, the method comprising: a) measuring in vitro the serum level of an MAA or an MSA in a blood sample taken from the subject; and b) comparing the serum level of the MAA or the MSA to a reference value associated with myositis in the subject, wherein if the serum level of the MAA or the MSA in the sample is greater than or equal to the reference value, then the subject is in need of further treatment with the second FcRn antagonist.

In some embodiments, the subject was previously treated with the first FcRn antagonist at a fixed dose of about 200 mg to about 20,000 mg or at a dose of about 2 mg/kg to about 200 mg/kg. In some embodiments, the subject was previously treated with the first FcRn antagonist at a fixed dose of 200 mg to 20,000 mg or at a dose of 2 mg/kg to 200 mg/kg.

In some embodiments, the subject was previously treated with the first FcRn antagonist at a dose of about 750 mg to about 3000 mg, administered subcutaneously.

In some embodiments, the subject was previously treated with the first FcRn antagonist at a dose of 750 mg to 3000 mg, administered subcutaneously.

In some embodiments, the subject was previously treated with the first FcRn antagonist at a dose of about 3 mg/kg to about 60 mg/kg, administered intravenously.

In some embodiments, the subject was previously treated with the first FcRn antagonist at a dose of 3 mg/kg to 60 mg/kg, administered intravenously.

In some embodiments, the subject was previously treated with the first FcRn antagonist at a dose of about 10 mg/kg to about 30 mg/kg, administered intravenously.

In some embodiments, the subject was previously treated with the first FcRn antagonist at a dose of 10 mg/kg to 30 mg/kg, administered intravenously.

In some embodiments, the effective amount of the second FcRn antagonist is a higher dose than the previous treatment with the first FcRn antagonist.

In some embodiments, the effective amount of the second FcRn antagonist is a lower dose than the previous treatment with the first FcRn antagonist.

In some embodiments, the effective amount of the second FcRn antagonist is administered at a fixed dose of about 200 mg to about 20,000 mg or at a dose of about 2 mg/kg to about 200 mg/kg. In some embodiments, the effective amount of the second FcRn antagonist is administered at a fixed dose of 200 mg to 20,000 mg or at a dose of 2 mg/kg to 200 mg/kg.

In some embodiments, the effective amount of the second FcRn antagonist is administered subcutaneously at a fixed dose of about 750 mg to about 3000 mg once weekly, once every two weeks, every three weeks, every four weeks, or every six weeks.

In some embodiments, the effective amount of the second FcRn antagonist is administered subcutaneously at a fixed dose of 750 mg to 3000 mg once weekly, once every two weeks, every three weeks, every four weeks, or every six weeks.

In some embodiments, the effective amount of the FcRn second antagonist is administered subcutaneously at a fixed dose of about 1000 mg or about 2000 mg once weekly, once every two weeks, every three weeks, every four weeks, or every six weeks.

In some embodiments, the effective amount of the FcRn second antagonist is administered subcutaneously at a fixed dose of 1000 mg or 2000 mg once weekly, once every two weeks, every three weeks, every four weeks, or every six weeks.

In some embodiments, the first FcRn antagonist and the second FcRn antagonist are each the same FcRn antagonist. In some embodiments, the FcRn antagonist comprises or consists of a variant Fc region or FcRn binding fragment thereof. In some embodiments, the variant Fc region or FcRn binding fragment thereof binds to FcRn with a higher affinity at pH 5.5 as compared to a corresponding wild-type Fc region. In some embodiments, the variant Fc region or FcRn binding fragment thereof binds to FcRn with a higher affinity at pH 6.0 and/or pH 7.4 as compared to a corresponding wild-type Fc region. In some embodiments, the variant Fc region comprises or consists of two Fc domains which form a homodimer or heterodimer. In some embodiments, the FcRn antagonist is an Fc region comprising amino acids Y, T, E, K, F, and Y at EU positions 252, 254, 256, 433, 434, and 436, respectively. In some embodiments, the FcRn antagonist is efgartigimod.

In some embodiments, the first FcRn antagonist and the second FcRn antagonist are each a different FcRn antagonist. In some embodiments, the first FcRn antagonist or the second FcRn antagonist comprises or consists of a variant Fc region or FcRn binding fragment thereof. In some embodiments, the variant Fc region or FcRn binding fragment thereof binds to FcRn with a higher affinity at pH 5.5 as compared to a corresponding wild-type Fc region. In some embodiments, the variant Fc region or FcRn binding fragment thereof binds to FcRn with a higher affinity at pH 6.0 and/or pH 7.4 as compared to a corresponding wild-type Fc region. In some embodiments, the variant Fc region comprises or consists of two Fc domains which form a homodimer or heterodimer. In some embodiments, the first FcRn antagonist or the second FcRn antagonist is an Fc region comprising amino acids Y, T, E, K, F, and Y at EU positions 252, 254, 256, 433, 434, and 436, respectively. In some embodiments, the first FcRn antagonist or the second FcRn antagonist is efgartigimod.

In some embodiments, the amino acid sequence of at least one of the Fc domains is selected from the group consisting of SEQ ID NO: 1, SEQ ID NO: 2, and SEQ ID NO: 3. In some embodiments, the amino acid sequence of both of the Fc domains is independently selected from the group consisting of SEQ ID NO: 1, SEQ ID NO: 2, and SEQ ID NO: 3.

In some embodiments, the first FcRn antagonist is an anti-FcRn antibody and the second FcRn antagonist is efgartigimod.

In some embodiments, the first FcRn antagonist is an anti-FcRn antibody and the second FcRn antagonist comprises the amino acid sequence of SEQ ID NO: 1, 2, or 3.

In some embodiments, the anti-FcRn antibody is rozanolixizumab, nipocalimab, orilanolimab, or batoclimab.

In some embodiments, the patient has not been previously treated with efgartigimod.

In some embodiments, the subject has one or more physical symptoms of myositis following treatment with the first FcRn antagonist.

In some embodiments, the subject has a serum level of an MAA or an MSA that is associated with relapse of myositis. In some embodiments, the MSA is selected from the group consisting of anti-SRP, anti-HMGCR, anti-Mi-2, anti-TIF1, anti-SAE, anti-NXP, anti-MDA5, anti-Jo-1, anti, PL-7, anti-PL-12, anti-EJ, and anti-OJ. In some embodiments, the MAA is selected from the group consisting of anti-PM/Scl 75, anti-Ku, anti-snRNP, anti-Ro52 (SSA), anti-Ro/60 (SSA), and anti-La (SSB).

In some embodiments, the myositis is selected from the group consisting of IMNM, DM, JDM, PM, and ASyS.

### BRIEF DESCRIPTION OF THE DRAWINGS

FIG. 1 is a schematic of a prophylactic efgartigimod treatment protocol in a mouse model of immune-mediated necrotizing myopathy (EIMNM). Groups of n=8 complement C5-deficient C57BL/10 Snj C5^{def} (C5^{-/-}) mice received daily intraperitoneal injections of IgG-depleted human serum as a source of human complement. To prevent xenoimmunization against human proteins, C5^{-/-} mice were pretreated with a single injection of cyclophosphamide (day −1). Disease was induced by injections of 2 mg IgG purified from an anti-HMGCR auto Ab+ IMNM patient or from a healthy donor as control (day 0 and 4). C5^{-/-} mice were treated in a preventive setting with subcutaneous injections of 20 mg/kg efgartigimod (day −1, 2, 4 and 6).

FIG. 2 is a set of graphs showing human IgG and anti-HMGCR titers. Human IgG titers were determined by ELISA (left panels) and anti-HMGCR⁺ autoantibody (aAb) titers were determined by ALBIA (right panels) in sera of C57BL/10 Snj C5^{def} (C5^{-/-}) mice receiving control IgG or anti-HMGCR⁺ IgG with or without efgartigimod. Levels of total IgG or anti-HMGCR IgG aAb were monitored in mouse serum by ELISA and ALBIA, respectively. Data are presented as mean±SD; ***p<0.005 by Mann-Whitney two-tailed test. Experiments were performed in triplicate (Exp 1-3).

FIG. 3 is a set of graphs showing grip strength (left panels) and muscle strength (right panels) in C57BL/10 Snj C5^{def} (C5^{-/-}) mice after injection of control IgG or anti-HMGCR⁺ IgG with or without efgartigimod. Muscle strength was assessed by measurement of gastrocnemius strength upon sciatic nerve electrostimulation (anesthetized animals). Data are presented as mean±SD; *p<0.05, **p<0.01 by Mann-Whitney two-tailed test. Experiments were performed in triplicate (Exp 1-3).

FIG. 4A is a set of four images showing extent of staining of human IgG (with anti-human IgG) in mouse muscle tissue. Upper left: negative control (mice not treated with human IgG); Upper right: mouse treated with total IgG from healthy donor; Lower left: mouse treated with total IgG from an IMNM patient positive for anti-HMGCR autoantibodies; Lower right: mouse treated with efgartigimod plus total IgG from an INM patient positive for anti-HMGCR autoantibodies. FIG. 4B is a set of four images showing extent of staining of nuclei (with DAPI) and human IgG (with anti-human IgG) in mouse muscle tissue. Upper left: negative control (mice not treated with human IgG); Upper right: mouse treated with total IgG from healthy donor; Lower left: mouse treated with total IgG from an IMNM patient positive for anti-HMGCR autoantibodies; Lower right: mouse treated with efgartigimod plus total IgG from an IMNM patient positive for anti-HMGCR autoantibodies.

FIG. 5A is a set of three images showing hematoxylin and eosin (H&E) staining of cryosections of mouse muscle tissue showing necrotic muscle fibers. Left: mouse treated with total IgG from healthy donor (control IgG); Middle: mouse treated with total IgG from an IMNM patient positive for anti-HMGCR autoantibodies (anti-HMGCR⁺ IgG); Right: mouse treated with efgartigimod plus total IgG from an IMNM patient positive for anti-HMGCR autoantibodies. Triangles indicate necrotic cells. FIG. 5B is graph showing the quantification of necrotic muscle fibers in C57BL/10 Snj C5^{def} (C5^{-/-}) mice after injection of control IgG or anti-HMGCR⁺ IgG with or without efgartigimod. Data are presented as mean±SD; *p<0.05, **p<0.01 by Mann-Whitney two-tailed test. FIG. 5C is graph showing the quantification of regenerating muscle fibers in C57BL/10 Snj C5^{def} (C5^{-/-}) mice after injection of control IgG or anti-HMGCR⁺ IgG with or without efgartigimod. Data are presented as mean±SD; *p<0.05, **p<0.01 by Mann-Whitney two-tailed test.

FIG. 6A-6B are sets of schematics of efgartigimod therapeutic treatment protocols in a mouse model of immune-mediated necrotizing myopathy (IMNM). Groups of n=8 Rag2 deficient (Rag2^{-/-}) C57/BL6 mice received daily intraperitoneal injections of IgG-depleted human serum as a source of human complement. The rationale for the use of Rag2^{-/-} mice was prevent xenoimmunization against human proteins. Disease was induced by injections of 2 mg IgG purified from an anti-HMGCR auto Ab+ IMNM patient or from a healthy donor as control (day 0, 4±day 8, 12, 16). Rag2^{-/-} mice were treated in a curative setting (day 8, 11, 15) after disease was induced by anti-HMGCR auto Ab+ IgG injections.

FIG. 7 is a graph showing reduction in human IgG titers in a mouse model of IMNM in response to therapeutic treatment with efgartigimod. Levels of total IgG were monitored in mouse serum by ELISA. Data are presented as mean±SD; **p<0.01, ***p<0.005 by Mann-Whitney two-tailed test.

FIG. 8 is a graph showing reduction in anti-HMGCR titers in a mouse model of IMNM in response to therapeutic treatment with efgartigimod. Levels of anti-HMGCR IgG aAb were monitored in mouse serum by ALBIA. Data are presented as mean±SD; **p<0.01, ***p<0.005 by Mann-Whitney two-tailed test.

FIG. 9 is a graph showing restoration of grip strength in a mouse model of IMNNM in response to therapeutic treatment with efgartigimod. Data are presented as mean±SD; *p<0.05, **p<0.01 by Mann-Whitney two-tailed test.

FIG. 10 is a graph showing restoration of muscle strength in a mouse model of IMNM in response to therapeutic treatment with efgartigimod. Data are presented as mean±SD; *p<0.05 by Mann-Whitney two-tailed test. Muscle strength was assessed by measurement of gastrocnemius strength upon sciatic nerve electrostimulation (anesthetized animals).

FIG. 11A is a set of four images showing extent of staining of human IgG (with anti-human IgG) in muscle tissue of mice in response to therapeutic treatment with efgartigimod. Upper left: negative control (mice not treated with human IgG); Upper right: mouse treated with total IgG from healthy donor; Lower left: mouse treated with total IgG from an IMNM patient positive for anti-HMGCR autoantibodies; Lower right: mouse treated with efgartigimod plus total IgG from an IMNM patient positive for anti-HMGCR autoantibodies. FIG. 11B is a set of four images showing extent of staining of nuclei (with DAPI) and human IgG (with anti-human IgG) in muscle tissue from mice in response to therapeutic treatment with efgartigimod. Upper left: negative control (mice not treated with human IgG); Upper right: mouse treated with total IgG from healthy donor; Lower left: mouse treated with total IgG from an IMNM patient positive for anti-HMGCR autoantibodies; Lower right: mouse treated with efgartigimod plus total IgG from an IMNM patient positive for anti-HMGCR autoantibodies.

FIG. 12 is a set of three images showing hematoxylin and eosin (H&E) staining of cryosections of muscle tissue showing necrotic and regenerating muscle fibers in mice in response to therapeutic treatment with efgartigimod. Left: mouse treated with total IgG from healthy donor (control IgG); Middle: mouse treated with total IgG from an IMNM patient positive for anti-HMGCR autoantibodies (anti-HMGCR⁺ IgG); Right: mouse treated with efgartigimod plus total IgG from an IMNM patient positive for anti-HMGCR autoantibodies. Triangles indicate necrotic cells; stars indicate regenerative cells.

FIG. 13 is a graph showing the quantification of necrotic muscle fibers in Rag2 deficient (Rag2^{-/-}) C57/BL6 mice after injection of control IgG or anti-HMGCR⁺ IgG with or without efgartigimod. Muscle biopsies were taken at day 18. Data are presented as mean±SD; *p<0.05, **p<0.01 by Mann-Whitney two-tailed test.

FIG. 14 is a graph showing the quantification of regenerating muscle fibers in Rag2 deficient (Rag2^{-/-}) C57/BL6 mice after injection of control IgG or anti-HMGCR⁺ IgG with or without efgartigimod. Muscle biopsies were taken at day 18. Data are presented as mean±SD; *p<0.05, **p<0.01 by Mann-Whitney two-tailed test.

### DETAILED DESCRIPTION

The present disclosure provides engineered FcRn antagonists and methods for their use in treating myositis, including immune-mediated necrotizing myopathy (IMNM), dermatomyositis (DM), polymyositis (PM), and antisynthetase syndrome (ASyS). Advantageously, the methods disclosed herein permit rapid treatment of disease, as well as the potential to taper and even discontinue corticosteroids after achieving clinical remission. The instant disclosure demonstrates that FcRn antagonists are highly effective in treating myositis. Treatment with FcRn antagonists significantly reduced myositis-specific autoantibodies and improved muscle strength within nine days in an animal model of immune-mediated necrotizing myopathy (IMNM), an idiopathic inflammatory myopathy.

### Definitions

As used herein, the term “FcRn” refers to a neonatal Fc receptor. Exemplary FcRn molecules include human FcRn encoded by the FCGRT gene as set forth in RefSeq NM 004107. The amino acid sequence of the corresponding protein is set forth in RefSeq NP_004098.

As used herein, the term “FcRn antagonist” refers to any agent that binds specifically to FcRn and inhibits the binding of immunoglobulin to FcRn (e.g., human FcRn). In an embodiment, the FcRn antagonist is an Fc region (e.g., a variant Fc region disclosed herein) that specifically binds to FcRn through the Fc region and inhibits the binding of immunoglobulin to FcRn. In an embodiment, the FcRn antagonist is not a full-length IgG antibody. In an embodiment, the FcRn antagonist comprises an antigen binding site that binds a target antigen and a variant Fc region. In an embodiment, the FcRn antagonist is an Fc fragment comprising or consisting of an Fc region and lacking an antigen binding site. In an embodiment, the term “FcRn antagonist” refers to an antibody or antigen-binding fragment thereof that specifically binds to FcRn via its antigen binding domain or via its Fc region and inhibits the binding of the Fc region of immunoglobulin (e.g., IgG autoantibodies) to FcRn.

As used herein, the terms “antibody” and “antibodies” include full-length antibodies, antigen-binding fragments of full-length antibodies, and molecules comprising antibody CDRs, VH regions, or VL regions. Examples of antibodies include monoclonal antibodies, recombinantly produced antibodies, monospecific antibodies, multi-specific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies (sdAb), monovalent antibodies, single chain antibodies or single-chain Fvs (scFv), camelid antibodies, affibody molecules, humanized antibodies, VHH fragments, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. Antibodies can be of any type (e.g., IgG, IgE, IgM, IgD, IgA, or IgY), any class (e.g., IgG₁, IgG₂, IgG₃, IgG₄, IgA₁ or IgA₂), or any subclass (e.g., IgG₂ₐ or IgG_{2b}) of immunoglobulin molecule.

As used herein, the term “Fc domain” refers to the portion of a single immunoglobulin heavy chain comprising both CH2 and CH3 domains of the antibody. In some embodiments, the Fc domain comprises at least a portion of a hinge (e.g., upper, middle, and/or lower hinge region) region, a CH2 domain, and a CH3 domain. In some embodiments, the Fc domain does not include the hinge region.

As used herein, the term “hinge region” refers to the portion of a heavy chain molecule that joins the CH1 domain to the CH2 domain. This hinge region comprises approximately 25 amino acid residues and is flexible, thus allowing the two N-terminal antigen binding regions to move independently. Hinge regions can be subdivided into three distinct domains: upper, middle, and lower hinge domains. The FcRn antagonists of the instant disclosure can include all or any portion of a hinge region. In some embodiments, the hinge region is from an IgG₁ antibody. In some embodiments, the hinge region comprises the amino acid sequence of EPKSCDKTHTCPPCP (SEQ ID NO: 12).

As used herein, the term “Fc region” refers to the portion of an immunoglobulin formed by the Fc domains of its two heavy chains. The Fc region can be a wild-type Fc region (native Fc region) or a variant Fc region. A native Fc region is homodimeric. The Fc region can be derived from any native immunoglobulin. In some embodiments, the is formed from an IgA, IgD, IgE, or IgG heavy chain constant region. In some embodiments, the Fc region is formed from an IgG heavy chain constant region. In some embodiments, the IgG heavy chain is an IgG₁, IgG₂, IgG₃ or IgG₄ heavy chain constant region. In some embodiments, the Fc region is formed from an IgG₁ heavy chain constant region. In some embodiments, the IgG₁ heavy chain constant region comprises a G1m1(a), qG1m2(x), G1m3(f), or G1m17(z) allotype. See, e.g., Jefferis and Lefranc (2009) mAbs 1(4): 332-338, and de Taeye et al. (2020) Front Immunol. 11:740, incorporated herein by reference in their entirety.

As used herein, the term “variant Fc region” refers to an Fc region with one or more alteration(s) relative to a native Fc region. Alterations can include amino acid substitutions, additions and/or deletions, linkage of additional moieties, and/or alteration of the native glycans. The term encompasses heterodimeric Fc regions where each of the constituent Fc domains is different. The term also encompasses single chain Fc regions where the constituent Fc domains are linked together by a linker moiety.

As used herein the term “FcRn binding fragment” refers to a portion of an Fc region that is sufficient to confer FcRn binding.

As used herein, the term “EU position” refers to the amino acid position in the EU numbering convention for the Fc region described in Edelman, G M et al., Proc. Natl. Acad. USA, 63, 78-85 (1969) and Rabat et al, in “Sequences of Proteins of Immunological Interest,” U.S. Dept. Health and Human Services, 5th edition, 1991.

As used herein, the term “baseline” refers to a measurement (e.g., a frequency of B cells, IgG levels) in a patient, e.g., in a patient's blood or urine, prior to the first administration (e.g., intravenous, or subcutaneous administration) of a treatment (e.g., an FcRn antagonist).

As used herein, the term “autoantibody-mediated disease” refers to any disease or disorder in which the underlying pathology is caused, at least in part, by pathogenic IgG autoantibodies.

As used herein, the term “treat,” “treating,” and “treatment” refer to therapeutic or preventative measures described herein. The methods of “treatment” employ administration of a polypeptide to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “dose” or “dosing” refers to an amount of an agent administered to a subject in a single administration.

As used herein, the terms “fixed dose” or “flat dose” both refer to a dose that does not vary based upon a characteristic (e.g., body mass, e.g., within a set range; sex; age, e.g., within a set range; etc.) of the subject.

As used herein, the term “prednisone equivalent dose” means a dose of prednisone or an equivalent dose of a systemic corticosteroid other than prednisone. Systemic corticosteroids are well-known and include compounds of various potencies and formulations. These are generally formulated as injectables or pills. Examples of commercially available systemic corticosteroids include, without limitation, betamethasone, cortisone, dexamethasone, hydrocortisone, methylprednisolone, prednisone, prednisolone, and triamcinolone.

As used herein, the term “remission” refers to a patient who has no new markers of an autoantibody-mediated disease and the baseline markers of the disease have completely resolved or healed.

As used herein, the term “relapse” or “flare” refers to a patient with an autoantibody-mediated disease who has an appearance of physical symptoms and/or an increase of a marker of the autoantibody-mediated disease after a period of remission of the autoantibody-mediated disease.

As used herein, the term “subject” or “patient” or “participant” includes any human or non-human animal. In an embodiment, the subject or patient or participant is a human or non-human mammal. In an embodiment, the subject or patient or participant is a human.

As used herein, the term “about” or “approximately” when referring to a measurable value, such as a dosage, encompasses variations of 20%, ±15%, +10%, +5%, +1%, or +0.1% of a given value or range, as are appropriate to perform the methods disclosed herein.

### Myositis

Myositis is a group of inflammatory disorders of the skeletal muscle also referred to as idiopathic inflammatory myopathies (IIM). This heterogeneous group of diseases includes many subtypes with varying pathologies. There is evidence that some JIM subtypes, specifically with immune-mediated necrotizing myopathy (IMNM), dermatomyositis (DM), and certain subtypes of polymyositis (PM) such as antisynthetase syndrome (ASyS), are likely driven by IgG autoantibodies (including myositis-specific antibodies [MSAs] and myositis-associated antibodies [MAAs]).

General symptoms of chronic IIM include slow but progressive muscle weakness. Inflammation damages the muscle fibers, which causes weakness, and may affect the arteries and blood vessels that pass through muscle. Other symptoms include fatigue after walking or standing, frequent episodes of tripping or falling, and difficulty swallowing or breathing. Some individuals may have muscle pain or muscles that are tender to touch.

Immune-mediated necrotizing myopathies (IMNM) represent a rare and relatively newly recognized distinct group of inflammatory myopathies. IMNM can occur at any age but usually affect adults. Symptoms are similar to polymyositis and dermatomyositis, with weakness in both the upper and lower body, difficulty rising from low chairs, climbing stairs, or lifting objects. However, the onset of these symptoms can be more severe and sudden, reaching their peak over a period of days or weeks. Other symptoms include fatigue, weight loss, and muscle pain. IMNM are characterized by high creatine kinase levels, and necrosis of skeletal muscle fibers with deposition of C5b-9 membrane attack complex (MAC). Most IMNM patients have autoantibodies (aAbs) directed against signal recognition particle (SRP) or hydroxy-3-methylglutaryl-CoaA reductase (HMGCR). In addition to their role as biomarkers, the relationship observed between aAb titers and disease severity suggests that these aAbs could be pathogenic and therefore the central players in EIMNM pathophysiology.

Polymyositis (PM) affects skeletal muscles and is rarely seen in children. Generally, onset occurs between age 30 and 60. Symptoms vary considerably from person to person, which can make it difficult to diagnose. Untreated progressive muscle weakness may lead to difficulty swallowing, speaking, rising from a sitting position, climbing stairs, lifting objects, or reaching overhead. Some people with polymyositis may also develop arthritis, shortness of breath, heart arrhythmias (irregular heartbeats), or congestive heart failure (when the heart is no longer able to pump out enough oxygen-rich blood). Antisynthetase syndrome (ASyS) is a polymyositis subtype associated with anti-tRNA synthetase autoantibodies (such as anti-Jo-1, anti-PL-7, anti-PL-12, anti-EJ, and anti-OJ) and one or more of Raynaud's phenomenon, arthritis, interstitial lung disease, fever that is not attributable to another cause, or evidence of myositis.

Dermatomyositis (DM) is characterized by a skin rash that precedes or accompanies progressive muscle weakness. The rash appears patchy and characteristically develops on the eyelids and on muscles used to extend or straighten joints (such as knuckles, elbows, knees, and toes). Rashes may also occur on the face, neck, shoulders, upper chest, back, and other locations and may be accompanied by swelling in the affected areas. One form the rashes take is called “heliotrope” (a purplish color) or lilac, but may also be red. It can occur around the eyes along with swelling, but also occurs on the upper chest or back what is called the “shawl” (around the neck) or “V-sign” above the breasts and may also occur on the face, upper arms, thighs, or hands. Another form the rash takes is called Gottron's sign which are red or violet, sometimes scaly, slightly raised papules that erupt on any of the finger joints (the metacarpophalangeal joints or the interphalangeal joints) The rash sometimes occurs without obvious muscle involvement and often becomes more evident with sun exposure.

Adults with dermatomyositis may experience weight loss or a low-grade fever, have inflamed lungs, and be sensitive to light. Adult dermatomyositis, unlike polymyositis, may accompany tumors of the breast, lung, female genitalia, or bowel. Children and adults with dermatomyositis may develop calcium deposits under the skin or in the muscle (calcinosis). Raised, reddish bumps (Gottron's papules) may appear on the knuckles. Calcinosis most often occurs one to three years after disease onset but may occur many years later.

Anti-TIF1 antibody (human transcriptional intermediary factor) is the most common autoantibody found in children with juvenile dermatomyositis. It is also found in adults with dermatomyositis, and high levels of the antibody are associated with an increased risk of cancer-associated DM in adults. Other autoantibodies specific for DM include anti-NXP (nuclear matrix protein) antibodies, anti-SAE (anti-small ubiquitin like modifier activating enzyme heterodimer) antibodies, and anti-IFN-induced melanoma differentiation-associated protein 5 (MDA5).

In some cases of polymyositis and dermatomyositis, distal muscles may be affected as the disease progresses. Polymyositis and dermatomyositis may be associated with collagen-vascular or autoimmune diseases such as lupus. Polymyositis may also be associated with infectious disorders such as HIV, which causes AIDS.

Anti-PM/Scl is associated with lung problems and an overlap of polymyositis and scleroderma. Anti-Ro/SSA antibodies (such as anti-Ro52 (SSA) and anti-Ro60 (SSA)) are the most prevalent MAA in myositis and frequently occurs together with anti-ARS antibodies or other MAAs (such as anti-Ku, anti-snRNP, and anti-La (SSB)).

Many patients with IIM have persistent impairment of muscle function, which leads to difficulties in daily life activities and a low health-related quality of life. The typical treatment for IIM is high-dose glucocorticoids combined with immunosuppressive drugs. The deleterious long-term effects of corticosteroids have been well established and include osteoporosis, cataracts, and weight gain. There are no therapies approved by the United States Food and Drug Administration (FDA) or the European regulatory authorities based on results of randomized controlled trials for IMNM and PM. Only one licensed treatment (10% intravenous immunoglobulin [IVIg]) is available for adults with DM that was approved based on results of randomized controlled clinical trials.

The total improvement score (TIS) is endorsed by the American College of Rheumatology [ACR] and European League Against Rheumatism [EULAR] to monitor JIM symptoms. This score is the weighted sum of improvement in 6 core set measures (CSMs) for disease activity: physician global assessment of disease activity (MDGA), patient global assessment of disease activity (PGA), manual muscle testing-8 (MMT8), health assessment questionnaire disability index (HAQ-DI), muscle enzymes, and extramuscular disease activity (assessed by the Myositis Disease Activity Assessment Tool [MDAAT]).

The TIS comprises CSMs scored both by the patient and the physician, considering the muscle manifestations and the extramuscular disease activity and physical function. It also includes objective disease activity parameters, i.e., the activity of the muscle enzymes. The International Myositis Assessment and Clinical Studies Group (IMACS) recommends the minimal TIS as the primary endpoint in IIM therapeutic studies. The criteria may be used as a continuous outcome measure, using the TIS, or as a categorical outcome of improvement (minimal, moderate, or major improvement). IMACS considers the threshold of minimal TIS to differentiate between treatment groups in clinical studies as clinically significant. To date, several phase 3 studies in patients with IIM have used the TIS as a primary endpoint (NCT02728752, NCT03981744, NCT04044690, and NCT04999020).

The absolute percentage change from baseline in each measure with varying weights is combined to obtain a TIS on a scale from 0 to 100. Higher scores indicate greater improvement. Improvements are defined by the following increases in TIS:
Minimal improvement—at least 20 points;
Moderate improvement—at least 40 points;
Major improvement—at least 60 points.

### MDGA

The MDGA is a tool that measures the physician's global evaluation of the participant's overall disease activity, defined as potentially reversible pathology or physiology resulting from IIM. The physician rates disease activity on the MDGA using a 10-cm VAS. Overall disease activity is rated by drawing a vertical mark on a 10-cm VAS from the left end of the line (no evidence of disease activity), midpoint of the line (moderate disease activity), and the right end of the line (extremely active or severe disease activity).

### PGA

The PGA is a tool that measures a patient's global evaluation of their overall disease activity at the time of assessment using a 10-cm VAS. The patient rates their overall disease activity by drawing a vertical mark on a 10-cm VAS from the left end of the line (no evidence of disease activity) to the right end of the line (extremely active or severe disease activity).

### MMT8

The MMT8 is a physician assessment of muscle strength in a set of 8 designated muscles tested bilaterally (proximal muscles [deltoids, biceps, gluteus maximus, gluteus medius, quadriceps] and distal muscles [wrist extensors and ankle dorsiflexors], potential score 0 to 140) and axially (neck flexors, potential score 0 to 10).

### HAQ-DI

The HAQ-DI assesses physical function and is recommended by IMACS as a CSM in therapeutic studies reporting on myositis disease activity. The patient assesses their usual abilities within the past week in 8 categories: dressing and grooming, arising, walking, reach, eating, hygiene, grip, and activities. Each category has 2 to 3 questions scored by the participant using a 4-point scale from 0 (without any difficulty) to 3 (unable to do). Scores may be adjusted based on the patient's responses to separate questions regarding their need for aids and devices or help from another person. A VAS may be included in the questionnaire. Respondents are asked to mark how much pain they have had because of their illness in the past week from 0 (no pain) to 100 (severe pain).

### Muscle Enzymes

Muscle-associated enzymes include creatine kinase (CK), alanine aminotransferase (ALT), aspartate aminotransaminase (AST), lactate dehydrogenase (LDH), and aldolase. These enzymes are measured in patient blood samples using validated methods. Reduction in prevalence of at least one of these enzymes is indicative of improvement in myositis.

### MDAAT

The MDAAT is a combined tool that includes the MYOACT, which is a series of physician's assessments of disease activity of various organ systems modified from the Vasculitis Activity Index, and the MITAX, which is modified from the British Isles Lupus Assessment Group approach to assess disease activity in lupus. Among the MYOACT components of the MDAAT assessments, the extramuscular global assessment is a CSM of the TIS and is a tool used by the physician for an overall evaluation of disease activity in all the extramuscular organ systems. This assessment specifically excludes muscle disease activity. The physician uses a 10-cm VAS to rate the participant's overall disease activity in all extramuscular systems within the past 4 weeks that are caused by active JIM disease. The MITAX component of the MDAAT assesses disease activity of various organ systems by assessing 26 clinical features within the past 4 weeks that are caused by active disease, using a 5 point scale: 0=not present in the past 4 weeks; 1=improving; 2=the same; 3=worse; 4=new.

### CDASI

CDASI is a skin-specific outcome measure used to assess disease in patients with DM. Disease in 15 different anatomical locations is rated using 3 activity measures (erythema, scale, erosion/ulceration) and 2 damage measures (poikiloderma, calcinosis). The 3 activity measures on the 15 anatomical locations can add up to 90 points; Gottron's papules on the hands, periungual, and alopecia can add up to 10 points. The 2 damage measures on the 15 anatomical locations can add up to 30 points with 2 additional points for Gottron's hands. CDASI also assesses the presence and severity of Gottron's papules, periungual changes, and alopecia. The resulting activity and damage scores range from 0 to 100 and 0 to 32, respectively. Higher scores indicate greater disease severity.

### FcRn Antagonists

FcRn antagonists that are useful in the methods and uses provided herein include any molecule that binds to and inhibits FcRn, including, but not limited to, any anti-FcRn antibody, any anti-FcRn binding region, or any Fc domain or Fc region.

In some embodiments, the FcRn antagonists disclosed herein comprise two, three, or four FcRn binding regions, such as an Fc region.

Any Fc region can be altered to produce a variant Fc region for use in the methods disclosed herein. In general, an Fc region, or FcRn-binding fragment thereof, is from a human immunoglobulin. It is understood, however, that the Fc region may be derived from an immunoglobulin of any other mammalian species, including for example, a camelid species, a rodent (e.g., a mouse, rat, rabbit, guinea pig) or non-human primate (e.g., chimpanzee, macaque) species. Moreover, the Fc region or portion thereof may be derived from any immunoglobulin class, including IgM, IgG, IgD, IgA, and IgE, and any immunoglobulin isotype, including IgG1, IgG₂, IgG₃, and IgG₄. In an embodiment, the Fc region is an IgG Fc region (e.g., a human IgG region). In an embodiment, the Fc region is an IgG1 Fc region (e.g., a human IgG1 region). In an embodiment, the Fc region is a chimeric Fc region comprising portions of several different Fc regions. Suitable examples of chimeric Fc regions are set forth in US 2011/0243966A1, which is incorporated herein by reference in its entirety. A variety of Fc region gene sequences (e.g., human constant region gene sequences) are available in the form of publicly accessible deposits.

An Fc region can be further truncated or internally deleted to produce a minimal FcRn-binding fragment thereof. The ability of an Fc-region fragment to bind to FcRn can be determined using any art recognized binding assay e.g., ELISA.

To enhance the manufacturability of the FcRn antagonists disclosed herein, it is preferable that the constituent Fc regions do not comprise any non-disulfide bonded cysteine residues. Accordingly, in an embodiment, the Fc regions do not comprise a free cysteine residue.

Any Fc variant, or FcRn-binding fragment thereof, that binds specifically to FcRn with increased affinity and reduced pH dependence relative to the native (i.e., wild-type) Fc region can be used in the methods disclosed herein. In an embodiment, the variant Fc region comprises amino acid alterations, substitutions, insertions and/or deletions that confer the desired characteristics. In an embodiment, the FcRn antagonist comprises or consists of a variant Fc region, or FcRn binding fragment thereof, which binds to FcRn with a higher affinity at pH 5.5 as compared to a corresponding wild-type Fc region. In some embodiments, the FcRn antagonist comprises or consists of a variant Fc region, or FcRn binding fragment thereof, which binds to FcRn with a higher affinity at pH 6.0 and/or at pH 7.4 as compared to a corresponding wild-type Fc region. In some embodiments, the FcRn antagonist comprises a variant Fc region, or FcRn binding fragment thereof, which binds to FcRn with a higher affinity at both acidic and neutral pH.

In some embodiments, the variant Fc region is derived from the Fc region of any native immunoglobulin. In some embodiments, the native immunoglobulin is a human immunoglobulin. In some embodiments, the immunoglobulin is IgA, IgD, IgE, or IgG. In some embodiments, the immunoglobulin is IgG. In some embodiments, the immunoglobulin is human IgA, human IgD, human IgE, or human IgG. In some embodiments, the immunoglobulin is human IgG. In some embodiments, the IgG is IgG1, IgG2, IgG3, or IgG4. In some embodiments, the human IgG is human IgG1, human IgG2, human IgG3, or human IgG4. In some embodiments, the variant Fc region varies from the human IgG₁ Fc region. In some embodiments, the human IgG1 Fc region comprises a G1m1(a), G1m2(x), G1m3(f), or G1m17(z) allotype.

In an embodiment, the variant Fc region, or FcRn binding fragment thereof consists of two Fc domains. In an embodiment, the FcRn antagonist is an Fc region comprising amino acids Y, T, E, K, F, and Y at EU positions 252, 254, 256, 433, 434, and 436, respectively.

In an embodiment, the variant Fc region comprises or consists of a first Fc domain and a second Fc domain which form a homodimer or heterodimer. In an embodiment, the first Fc domain and/or the second Fc domain comprise amino acids Y, T, E, K, and F at EU positions 252, 254, 256, 433, and 434, respectively. In an embodiment, the first Fc domain and/or the second Fc domain comprise amino acids Y, T, E, K, F, and Y at EU positions 252, 254, 256, 433, 434, and 436, respectively.

In an embodiment, the first Fc domain and/or the second Fc domain comprise an amino acid sequence independently selected from the group consisting of SEQ ID NO: 1, SEQ ID NO: 2, and SEQ ID NO: 3. In an embodiment, the first Fc domain and the second Fc domain comprise an amino acid sequence independently selected from the group consisting of SEQ ID NO: 1, SEQ ID NO: 2, and SEQ ID NO: 3.

In an embodiment, the amino acid sequence of the Fc domains of the variant Fc region comprises the amino acid sequence of SEQ ID NO: 1. In an embodiment, the amino acid sequence of the Fc domains of the variant Fc region consists of the amino acid sequence of SEQ ID NO: 1. In an embodiment, the amino acid sequence of the Fc domains of the variant Fc region comprises the amino acid sequence of SEQ ID NO: 2. In an embodiment, the amino acid sequence of the Fc domains of the variant Fc region consists of the amino acid sequence of SEQ ID NO: 2. In an embodiment, the amino acid sequence of the Fc domains of the variant Fc region comprises the amino acid sequence of SEQ ID NO: 3. In an embodiment, the amino acid sequence of the Fc domains of the variant Fc region consists of the amino acid sequence of SEQ ID NO: 3.

In an embodiment, the FcRn antagonist consists of a variant Fc region, wherein the variant Fc region comprises two Fc domains, wherein the amino acid sequence of each of the Fc domains is independently selected from SEQ ID NO: 1, SEQ ID NO: 2, or SEQ ID NO: 3.

In certain embodiments, the variant Fc region is a heterodimer, where the constituent Fc domains are different from each other. Methods of producing Fc heterodimers are known in the art (see e.g., U.S. Pat. No. 8,216,805, which is incorporated by reference herein in its entirety). In an embodiment, the FcRn antagonist consists of a variant Fc region, wherein the variant Fc region consists of two Fc domains which form a heterodimer, wherein the amino acid sequence of each of the Fc domains is independently selected from SEQ ID NO: 1, SEQ ID NO: 2, or SEQ ID NO: 3. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 1 and the amino acid sequence of the second Fc domain consists of or comprises the amino acid sequence of SEQ ID NO: 2 or SEQ ID NO: 3. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 2 and the amino acid sequence of the second Fc domain consists of or comprises the amino acid sequence of SEQ ID NO: 1 or SEQ ID NO: 3. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 3 and the amino acid sequence of the second Fc domain consists of or comprises the amino acid sequence of SEQ ID NO: 1 or SEQ ID NO: 2.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 1.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 2.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 3.

In an embodiment, the FcRn antagonist is efgartigimod (CAS Registry No. 1821402-21-4). The term “efgartigimod” as used herein is interchangeable with “efgartigimod-alfa.” In some embodiments, efgartigimod is efgartigimod-alfa fcab.

TABLE 1
Amino acid sequences of variant Fc regions

| SEQ | |
|---|---|
| ID | |
| NO: | Amino Acid Sequence |
| 1 | CPPCPAPELLGGPSVFLFPPKPKDTLYITREPEVTCVVVDVSHED |
| | PEVKFNWYVDGVEVHNAKTKPREEQYNSTYRVVSVLTVLHQDWLN |
| | GKEYKCKVSNKALPAPIEKTISKAKGQPREPQVYTLPPSRDELTK |
| | NQVSLTCLVKGFYPSDIAVEWESNGQPENNYKTTPPVLDSDGSFF |
| | LYSKLTVDKSRWQQGNVFSCSVMHEALKFHYTQKSLSLSPG |
| 2 | DKTHTCPPCPAPELLGGPSVFLFPPKPKDTLYITREPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSR |
| | DELTKNQVSLTCLVKGFYPSDIAVEWESNGQPENNYKTTPPVLDS |
| | DGSFFLYSKLTVDKSRWQQGNVFSCSVMHEALKFHYTQKSLSLSP |
| | GK |
| 3 | DKTHTCPPCPAPELLGGPSVFLFPPKPKDTLYITREPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSR |
| | DELTKNQVSLTCLVKGFYPSDIAVEWESNGQPENNYKTTPPVLDS |
| | DGSFFLYSKLTVDKSRWQQGNVFSCSVMHEALKFHYTQKSLSLSP |
| | G |

In an embodiment, the anti-FcRn antibody is rozanolixizumab (UCB7665), nipocalimab (M281), orilanolimab (ALXN1830/SYNT001), or batoclimab (IMVT-1401/RVT1401/IBM9161).

In an embodiment, an antibody that binds specifically to FcRn and inhibits the binding of the Fc region of immunoglobulin to FcRn is nipocalimab, also known as M281. Nipocalimab is a full-length “Fc dead” IgG1 monoclonal antibody. Nipocalimab has been administered as an intravenous infusion in Phase 2 clinical trials for the treatment of myasthenia gravis (MG), warm autoimmune hemolytic anemia (WAIHA), and hemolytic disease of fetus and newborn (HDFN). Nipocalimab comprises the light chain (SEQ ID NO: 4) and heavy chain (SEQ ID NO: 5) sequences set forth in Table 2 below:

TABLE 2
Heavy chain and light chain sequences of
nipocalimab

| SEQ | |
|---|---|
| ID | |
| NO: | Amino Acid Sequence |
| 4 | QSALTQPASVSGSPGQSITISCTGTGSDVGSYNLVSWYQQHPGKA |
| | PKLMIYGDSERPSGVSNRFSGSKSGNTASLTISGLQAEDEADYYC |
| | SSYAGSGIYVFGTGTKVTVLGQPKAAPSVTLFPPSSEELQANKAT |
| | LVCLISDFYPGAVTVAWKADSSPVKAGVETTTPSKQSNNKYAASS |
| | YLSLTPEQWKSHKSYSCQVTHEGSTVEKTVAPTECS |
| 5 | EVQLLESGGGLVQPGGSLRLSCAASGFTFSTYAMGWVRQAPGKGL |
| | EWVSSIGASGSQTRYADSVKGRFTISRDNSKNTLYLQMNSLRAED |
| | TAVYYCARLAIGDSYWGQGTMVTVSSASTKGPSVFPLAPSSKSTS |
| | GGTAALGCLVKDYFPEPVTVSWNSGALTSGVHTFPAVLQSSGLYS |
| | LSSVVTVPSSSLGTQTYICNVNHKPSNTKVDKKVEPKSCDKTHTC |
| | PPCPAPELLGGPSVFLFPPKPKDTLMISRTPEVTCVVVDVSHEDP |
| | EVKFNWYVDGVEVHNAKTKPREEQYASTYRVVSVLTVLHQDWLNG |
| | KEYKCKVSNKALPAPIEKTISKAKGQPREPQVYTLPPSREEMTKN |
| | QVSLTCLVKGFYPSDIAVEWESNGQPENNYKTTPPVLDSDGSFFL |
| | YSKLTVDKSRWQQGNVFSCSVMHEALHNHYTQKSLSLSPG |

In an embodiment, an antibody that binds specifically to FcRn and inhibits the binding of the Fc region of immunoglobulin to FcRn is rozanolixizumab, also known as UCB 7665. Rozanolixizumab is a full-length humanized IgG₄ monoclonal antibody. Rozanolixizumab has been administered as a subcutaneous infusion in ongoing clinical trials for MG, immune thrombocytopenia (FTP), and chronic inflammatory demyelinating polyneuropathy (CIDP). Rozanolixizumab comprises the light chain (SEQ ID NO: 6) and heavy chain (SEQ ID NO: 7) sequences set forth in Table 3 below:

TABLE 3
Heavy chain and light chain sequences of
rozanolixizumab

| SEQ | |
|---|---|
| ID | |
| NO: | Amino Acid Sequence |
| 6 | DIQMTQSPSSLSASVGDRVTITCKSSQSLVGASGKTYLYWLFQKP |
| | GKAPKRLIYLVSTLDSGIPSRFSGSGSGTEFTLTISSLQPEDFAT |
| | YYCLQGTHFPHTFGQGTKLEIKRTVAAPSVFIFPPSDEQLKSGTA |
| | SVVCLLNNFYPREAKVQWKVDNALQSGNSQESVTEQDSKDSTYSL |
| | SSTLTLSKADYEKHKVYACEVTHQGLSSPVTKS FNRGEC |
| 7 | EVPLVESGGGLVQPGGSLRLSCAVSGFTFSNYGMVWVRQAPGKGL |
| | EWVAYIDSDGDNTYYRDSVKGRFTISRDNAKSSLYLQMNSLRAED |
| | TAVYYCTTGIVRPFLYWGQGTLVTVSSASTKGPSVFPLAPCSRST |
| | SESTAALGCLVKDYFPEPVTVSWNSGALTSGVHTFPAVLQSSGLY |
| | SLSSVVTVPSSSLGTKTYTCNVDHKPSNTKVDKRVESKYGPPCPP |
| | CPAPEFLGGPSVFLFPPKPKDTLMISRTPEVTCVVVDVSQEDPEV |
| | QFNWYVDGVEVHNAKTKPREEQFNSTYRVVSVLTVLHQDWLNGKE |
| | YKCKVSNKGLPSSIEKTISKAKGQPREPQVYTLPPSQEEMTKNQV |
| | SLTCLVKGFYPSDIAVEWESNGQPENNYKTTPPVLDSDGSFFLYS |
| | RLTVDKSRWQEGNVFSCSVMHEALHNHYTQKSLSLSLGK |

In an embodiment, an antibody that binds specifically to FcRn and inhibits the binding of the Fe region of immunoglobulin to FcRn is orilanolimab, also known as SYNT001. Orilanolimab is another full-length humanized IgG₄ monoclonal antibody. Orilanolimab has been administered as an intravenous infusion in Phase 2 clinical trials for treatment of WAIHA. Orilanolimab comprises the light chain (SEQ ID NO: 8) and heavy chain (SEQ TD NO: 9) sequences set forth in Table 4 below:

TABLE 4
Heavy chain and light chain sequences of
orilanolimab

| SEQ | |
|---|---|
| ID | |
| NO: | Amino Acid Sequence |
| 8 | DIQMTQSPSSLSASVGDRVTITCKASDHINNWLAWYQQKPGQAPR |
| | LLISGATSLETGVPSRFSGSGTGKDYTLTISSLQPEDFATYYCQQ |
| | YWSTPYTFGGGTKVEIKRTVAAPSVFIFPPSDEQLKSGTASVVCL |
| | LNNFYPREAKVQWKVDNALQSGNSQESVTEQDSKDSTYSLSSTLT |
| | LSKADYEKHKVYACEVTHQGLSSPVTKSFNRGEC |
| 9 | QVQLVQSGAELKKPGASVKLSCKASGYTFTSYGISWVKQATGQGL |
| | EWIGEIYPRSGNTYYNEKFKGRATLTADKSTSTAYMELRSLRSED |
| | SAVYFCARSTTVRPPGIWGTGTTVTVSSASTKGPSVFPLAPCSRS |
| | TSESTAALGCLVKDYFPEPVTVSWNSGALTSGVHTFPAVLQSSGL |
| | YSLSSVVTVPSSSLGTKTYTCNVDHKPSNTKVDKRVESKYGPPCP |
| | PCPAPEFLGGPSVFLFPPKPKDTLMISRTPEVTCVVVDVSQEDPE |
| | VQFNWYVDGVEVHNAKTKPREEQFNSTYRVVSVLTVLHQDWLNGK |
| | EYKCKVSNKGLPSSIEKTISKAKGQPREPQVYTLPPSQEEMTKNQ |
| | VSLTCLVKGFYPSDIAVEWESNGQPENNYKTTPPVLDSDGSFFLY |
| | SRLTVDKSRWQEGNVFSCSVM HEALHNHYTQKSLSLSLG |

In an embodiment, an antibody that binds specifically to FcRn and inhibits the binding of the Fc region of immunoglobulin to FcRn is batoclimab, also known as IMVT1401/RVT1401/HBM9161. Batoclimab is another full-length “Fc dead” IgG1 monoclonal antibody. Batoclimab has been administered as a subcutaneous injection in ongoing Phase 2 clinical trials for treatment of MG and Graves' ophthalmopathy. Batoclimab comprises the light chain (SEQ ID NO: 10) and heavy chain (SEQ ID NO: 11) sequences set forth in Table 5 below:

TABLE 5
Heavy chain and light chain sequences of
batoclimab

| SEQ | |
|---|---|
| ID | |
| NO: | Amino Acid Sequence |
| 10 | SYVLTQSPSVSVAPGQTARITCGGNNIGSKSVHWYQQKPGQAPVL |
| | VVYDDSDRPSGIPERFSASNSGNTATLTISRVEAGDEADYYCQVW |
| | DSSSDHVVFGGGTKLTVLGQPKAAPSVTLFPPSSEELQANKATLV |
| | CLISDFYPGAVTVAWKADSSPVKAGVETTTPSKQSNNKYAASSYL |
| | SLTPEQWKSHRSYSCQVTHEGSTVEKTVAPTECS |
| 11 | QLLLQESGPGLVKPSETLSLTCTVSGGSLSSSFSYWVWIRQPPGK |
| | GLEWIGTIYYSGNTYYNPSLKSRLTISVDTSKNHFSLKLSSVTAA |
| | DTAVYYCARRAGILTGYLDSWGQGTLVTVSSASTKGPSVFPLAPS |
| | SKSTSGGTAALGCLVKDYFPEPVTVSWNSGALTSGVHTFPAVLQS |
| | SGLYSLSSVVTVPSSSLGTQTYICNVNHKPSNTKVDKRVEPKSCD |
| | KTHTCPPCPAPEAAGGPSVFLFPPKPKDTLMISRTPEVTCVVVDV |
| | SHEDPEVKFNWYVDGVEVHNAKTKPREEQYNSTYRVVSVLTVLHQ |
| | DWLNGKEYKCKVSNKALPAPIEKTISKAKGQPREPQVYTLPPSRE |
| | EMTKNQVSLTCLVKGFYPSDIAVEWESNGQPENNYKTTPPVLDSD |
| | GSFFLYSKLTVDKSRWQQGNV FSCSVMHEALHNHYTQKSLSLSP |
| | G |

### Pharmaceutical Compositions

In an aspect, the instant disclosure provides pharmaceutical compositions comprising an FcRn antagonist for use in methods of treating myositis. In certain embodiments, these compositions comprise or consist of a variant Fc region, or FcRn-binding fragment thereof, that binds specifically to FcRn, particularly human FcRn, with increased affinity and reduced pH dependence relative to a native Fc region. In other embodiments, the FcRn antagonist composition is an antibody or antigen-binding fragment thereof that binds specifically to FcRn via its antigen binding domain and inhibits the binding of Fc region of immunoglobulin to FcRn. In general, these FcRn antagonists inhibit the binding of Fc-containing agents (e.g., antibodies and immunoadhesins) to FcRn in vivo, which results in an increased rate of degradation of the Fc-containing agents and, concomitantly, a reduced serum level of these agents.

In an embodiment, the FcRn antagonist is efgartigimod. Efgartigimod (ARGX-113) is a modified human immunoglobulin (Ig) gamma (IgG) 1-derived Fc of the za allotype that binds with nanomolar affinity to human FcRn. Efgartigimod encompasses the IgG1 Fc region (encompassing residues of SEQ ID NO: 2) and has been engineered using ABDEG™ technology to increase its affinity for FcRn at both physiological and acidic pH. Vaccaro C et al., Nat Biotechnol. 2005; 23(10):1283. See also U.S. Pat. No. 10,316,073, the contents of which is incorporated by reference herein in its entirety. The increased affinity for FcRn of efgartigimod at both acidic and physiological pH results in a blockage of FcRn-mediated recycling of IgGs.

Efgartigimod has a molecular weight of about 54 kDa, which is about one-third the molecular weight of full-length IgG (MW ca. 150 kDa). Thus, 10 mg efgartigimod is about 185 nmol, such that a dose of 10 mg efgartigimod/kg body weight corresponds to about 185 nmol efgartigimod/kg body weight, and a dose of 25 mg efgartigimod/kg of body weight corresponds to about 462.5 nmol efgartigimod/kg body weight. In contrast, a dose of 10 mg full-length IgG antibody/kg body weight corresponds to about 67 nmol/kg body weight. Furthermore, a 1000 mg fixed dose of efgartigimod corresponds to a fixed dose of about 18500 nmol of efgartigimod while a 2000 mg fixed dose of efgartigimod corresponds to a fixed dose of about 37,000 nmol of efgartigimod.

Due to its increased affinity for FcRn at both acidic and neutral pH, efgartigimod blocks the FcRn/IgG complex from forming, which results in degradation of endogenous IgGs, including autoantibodies that cause IgG-mediated autoimmune diseases. This blocking of FcRn by efgartigimod results in a rapid and profound reduction in autoantibody levels, which underlies the therapeutic strategy for the treatment of autoimmune indications where IgG autoantibodies are expected to have a central role in the disease pathology, e.g., conditions such as myositis.

Efgartigimod is under development for both the intravenous (IV) and subcutaneous (SC) administration route. For SC administration, in certain embodiments, efgartigimod may be administered alone. Alternatively, for SC administration, in certain embodiments, efgartigimod may be administered co-formulated with hyaluronidase, for example, in particular, rHuPH20. The co-formulated material will allow SC dosing of larger volumes.

rHuPH20 is the active ingredient of Halozyme's commercial product HYLENEX® recombinant (hyaluronidase human injection), referred to as HYLENEX®, which was approved by FDA for marketed use in the U.S. in December 2005. HYLENEX® is a tissue permeability modifier indicated as an adjuvant in SC fluid administration for achieving hydration, to increase the dispersion and absorption of other injected drugs, and in SC urography, for improving resorption of radiopaque agents.

rHuPH20 is a recombinant enzyme human hyaluronidase produced by genetically engineered Chinese hamster ovary (CHO) cells containing a deoxyribonucleic plasmid encoding a soluble fragment of human hyaluronidase (posterior head protein 20 [PH20]).

The HZ202 rHuPH20 DS is currently registered in HYLENEX® and other biologic drug products co-formulated with rHuPH20 DS. As such, in certain embodiments HZ202 rHuPH20 DS is used in the efgartigimod/rHuPH20 co-formulated product for SC administration (i.e., efgartigimod PH20 SC).

SC injection volumes are typically limited to 2.5 mL due to concerns regarding injection pain associated with larger volumes. It has been demonstrated that rHuPH20 offers a solution to the volume limitation associated with fast SC injections. rHuPH20 acts locally and transiently to depolymerize hyaluronan, a gel-like substance found in the subcutaneous layer of the skin. This results in decreased resistance to fluid flow and may increase dispersion and absorption of injected medicines and fluids, allowing for larger volume to be injected with limited swelling or pain. It has been shown that rHuPH20 allows for the fast absorption of a relatively large volume (10 mL) when administered SC. Shpilberg O et al., Br J Cancer. 2013; 109(6):1556-1561. Very little injection site swelling was observed when 10 mL of IgG solution was administered SC using rHuPH20 at 2000 U/mL, whereas a large injection site swelling was observed when 10 mL of IgG solution was injected without rHuPH20. Shpilberg O et al., Br J Cancer. 2013; 109(6):1556-1561.

rHuPH20 is transiently acting and is not systematically absorbed. It has been demonstrated to exert no long-term local effects. rHuPH20 has a half-life in the skin of less than 30 minutes. Hyaluronan levels in subcutaneous tissues return to normal within 24 to 48 hours because of the rapid natural turnover of hyaluronan.

rHuPH20 is approved for SC administration in co-formulations with other active ingredients (RITUXAN HYCELA®/MABTHERA® SC [rituximab] for Non-Hodgkin's lymphoma (NHL) and chronic lymphocytic leukemia (CLL) and HERCEPTIN HYLECTATM/HERCEPTIN® SC [trastuzumab]) in the US and Europe with an enzyme concentration of 2000 U/mL and an injectable volume that ranges from 5 to 13.4 mL.

In some embodiments, the pharmaceutical formulation comprises an FcRn antagonist in an amount from about 20 mg to about 20,000 mg. In some embodiments, the pharmaceutical formulation comprises an FcRn antagonist in an amount from about 200 mg to about 20,000 mg. In some embodiments, the pharmaceutical formulation comprises an FcRn antagonist in an amount from about 300 mg to about 6000 mg. In some embodiments, the pharmaceutical formulation comprises an FcRn antagonist in an amount from about 750 mg to about 3000 mg. In some embodiments, the pharmaceutical formulation comprises an FcRn antagonist in an amount from about 1000 mg to about 2500 mg. In some embodiments, the pharmaceutical formulation comprises an FcRn antagonist in an amount from about 1000 mg to about 2000 mg.

In some embodiments, the pharmaceutical formulation comprises an FcRn antagonist in an amount from 20 mg to 20,000 mg. In some embodiments, the pharmaceutical formulation comprises an FcRn antagonist in an amount from 200 mg to 20,000 mg. In some embodiments, the pharmaceutical formulation comprises an FcRn antagonist in an amount from 300 mg to 6000 mg. In some embodiments, the pharmaceutical formulation comprises an FcRn antagonist in an amount from 750 mg to 3000 mg. In some embodiments, the pharmaceutical formulation comprises an FcRn antagonist in an amount from 1000 mg to 2500 mg. In some embodiments, the pharmaceutical formulation comprises an FcRn antagonist in an amount from 1000 mg to 2000 mg.

In some embodiments, the pharmaceutical formulation comprises about 1000 mg or about 2000 mg of an FcRn antagonist. In some embodiments, the pharmaceutical formulation comprises 1000 mg or 2000 mg of an FcRn antagonist. In some embodiments, the FcRn antagonist is efgartigimod.

In some embodiments, the pharmaceutical formulation comprises efgartigimod in an amount from about 800 mg to about 1200 mg. In some embodiments, the pharmaceutical formulation comprises about 1000 mg efgartigimod. In some embodiments, the pharmaceutical formulation comprises 1000 mg efgartigimod.

In some embodiments, the pharmaceutical formulation comprises from about 10 mg/mL to about 200 mg/mL efgartigimod. In some embodiments, the pharmaceutical formulation comprises from 10 mg/mL to 200 mg/mL efgartigimod.

In some embodiments, the pharmaceutical formulation comprises about 20 mg/mL efgartigimod. In some embodiments, the pharmaceutical formulation comprises 20 mg/mL efgartigimod.

In some embodiments, the pharmaceutical formulation comprises about 180 mg/ml efgartigimod. In some embodiments, the pharmaceutical formulation comprises 180 mg/ml efgartigimod.

In some embodiments, the pharmaceutical formulation further comprises hyaluronidase. In some embodiments, the hyaluronidase is recombinant human hyaluronidase PH20 (rHuPH20).

The hyaluronidase can be present in the pharmaceutical formulation in any suitable amount. In an embodiment, the amount of hyaluronidase enzyme is from about 1000 U/ml to about 3000 U/ml. In an embodiment, the amount of hyaluronidase enzyme is about 1000 U/mL, about 1500 U/mL, about 2000 U/mL, about 2500 U/mL, or about 3000 U/mL. In an embodiment, the amount of hyaluronidase enzyme is 2000 U/mL.

In some embodiments, the rHuPH20 is present in the pharmaceutical formulation in an amount of about 11,000 U. In some embodiments, the rHuPH20 is present in the pharmaceutical formulation in an amount of 11,000 U.

In any of the above embodiments, the pharmaceutical formulation may be a unit dosage form.

In an embodiment, the unit dosage form comprises the FcRn antagonist as a dry formulation for dissolution such as a lyophilized powder, freeze-dried powder, or water-free concentrate. In an embodiment, the dry formulation is comprised in a hermetically sealed container such as a vial, an ampoule, or a sachet.

In an embodiment, the unit dosage form comprises the FcRn antagonist as a liquid formulation, e.g., injection or infusion solution. In an embodiment, the liquid formulation is comprised in a hermetically sealed container such as a vial, a sachet, a pre-filled syringe, a pre-filled autoinjector, or a cartridge for a reusable syringe or applicator.

In an embodiment, the unit dosage per vial may contain 0.5 ml, 1 ml, 2 ml, 3 ml, 4 ml, 5 ml, 6 ml, 7 ml, 8 ml, 9 ml, 10 ml, 15 ml, or 20 ml of an FcRn antagonist ranging from about 500 to about 2500 mg or from about 1000 mg to about 2000 mg. In an embodiment, these preparations can be adjusted to a desired concentration by adding a sterile diluent to each vial.

The formulations disclosed herein include bulk drug compositions useful in the manufacture of pharmaceutical compositions (e.g., compositions that are suitable for administration to a subject or patient) which can be used in the preparation of unit dosage forms. In an embodiment, a composition of the invention is a pharmaceutical composition. Such compositions comprise a prophylactically or therapeutically effective amount of one or more prophylactic or therapeutic agents (e.g., an FcRn antagonist of the invention or other prophylactic or therapeutic agent), and a pharmaceutically acceptable carrier. In an embodiment, the pharmaceutical compositions are formulated to be suitable for subcutaneous administration to a subject.

### Methods

In an aspect, methods for treating myositis using an FcRn antagonist are provided. In certain embodiments, the myositis is one or more of the following: immune-mediated necrotizing myopathy (IMNM), dermatomyositis (DM), juvenile dermatomyositis (JDM), polymyositis (PM), and antisynthetase syndrome (ASyS). In certain embodiments, the myositis is IMNM. In certain embodiments, the FcRn antagonist is efgartigimod. An important goal and feature of the methods disclosed herein is the reduction or even the elimination of the use of potentially toxic agents such as corticosteroids (e.g., prednisone) in the treatment of myositis. Another important goal and feature of the methods disclosed herein is rapid onset of disease control. Yet another important goal and feature of the methods disclosed herein is achievement of long-lasting complete remission on minimal treatment, preferably without the use of potentially toxic agents such as corticosteroids (e.g., prednisone). Effective treatment of myositis using an FcRn antagonist may include at least one of the elements of the group consisting of: muscle strength improvement, muscle strength stabilization, QoL (Quality of Life) improvement, pulmonary function improvement, pulmonary function stabilization, safety and/or tolerability of the FcRn antagonist, steroid sparing effect and pain improvement.

In certain embodiments, the myositis may be characterized as severe or non-severe as classified by the physician's global evaluation of disease activity (MDGA). In certain embodiments, the myositis may be characterized as severe myositis (e.g., MDGA score of ≥5). In other embodiments, the myositis may be characterized as non-severe myositis (e.g., MDGA score of <5).

In some embodiments, the myositis may by characterized by an MMT8 score of <142/150. In some embodiments, the myositis may be characterized by one or more of the following: a MDGA score of ≥2, a PGA score of ≥2, an extramuscular global score of ≥2, an HAQ-DI score of ≥0.25, and muscle enzyme (such as CK, aldolase, lactate dehydrogenase (LDH), AST, or ALT) ≥1.5 times the upper limit of normal.

In some embodiments, the myositis is IMNM and may be characterized as anti-signal recognition particle (SRP) positive, anti-3-hydroxy-3-methylglutaryl coenzyme A reductase (HMGCR) positive, or both anti-SRP positive and anti-HMGCR positive.

In some embodiments, the myositis is PM (including ASyS) and may be characterized as positive for at least one anti-aminoacyl-tRNA synthetase (such as anti-Jo-1, anti-PL-7, anti-PL-12, anti-EJ, and/or anti-OJ antibodies).

In some embodiments, the myositis is DM or JDM and may be characterized by active DM skin rash (such as Gottron's papules, Gottron's signs, and/or heliotrope rash).

In some embodiments, the FcRn antagonist is administered at a fixed dose of about 20 mg to about 20,000 mg. In some embodiments, the FcRn antagonist is administered at a fixed dose of about 200 mg to about 20,000 mg. In some embodiments, the FcRn antagonist is administered at a fixed dose of about 300 mg to about 6000 mg. In some embodiments, the FcRn antagonist is administered at a fixed dose of about 750 mg to about 3000 mg. In some embodiments, the FcRn antagonist is administered at a fixed dose of about 1000 mg to about 2500 mg. In some embodiments, the FcRn antagonist is administered at a fixed dose of about 1000 mg to about 2000 mg. In some embodiments, the FcRn antagonist is efgartigimod.

In some embodiments, the FcRn antagonist is administered at a fixed dose of 20 mg to 20,000 mg. In some embodiments, the FcRn antagonist is administered at a fixed dose of 200 mg to 20,000 mg. In some embodiments, the FcRn antagonist is administered at a fixed dose of 300 mg to 6000 mg. In some embodiments, the FcRn antagonist is administered at a fixed dose of 750 mg to 3000 mg. In some embodiments, the FcRn antagonist is administered at a fixed dose of 1000 mg to 2500 mg. In some embodiments, the FcRn antagonist is administered at a fixed dose of 1000 mg to 2000 mg. In some embodiments, the FcRn antagonist is efgartigimod.

In some embodiments, the FcRn antagonist is administered at a fixed dose of about 20 mg, about 50 mg, about 100 mg, about 200 mg, about 250 mg, about 300 mg, about 500 mg, about 750 mg, about 1000 mg, about 1500 mg, about 2000 mg, about 2500 mg, about 3000 mg, about 4000 mg, about 5000 mg, about 6000 mg, about 7000 mg, about 8000 mg, about 9000 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg. In some embodiments, the FcRn antagonist is efgartigimod.

In some embodiments, the FcRn antagonist is administered at a fixed dose of 20 mg, 50 mg, 100 mg, 200 mg, 250 mg, 300 mg, 500 mg, 750 mg, 1000 mg, 1500 mg, 2000 mg, 2500 mg, 3000 mg, 4000 mg, 5000 mg, 6000 mg, 7000 mg, 8000 mg, 9000 mg, 10,000 mg, 11,000 mg, 12,000 mg, 13,000 mg, 14,000 mg, 15,000 mg, 16,000 mg, 17,000 mg, 18,000 mg, 19,000 mg, or 20,000 mg. In some embodiments, the FcRn antagonist is efgartigimod.

In some embodiments, the FcRn antagonist is administered at a dose of about 0.2 mg/kg to about 200 mg/kg. In some embodiments, the FcRn antagonist is administered at a dose of about 2 mg/kg to about 200 mg/kg. In some embodiments, the FcRn antagonist is administered at a dose of about 2 mg/kg to about 120 mg/kg. In some embodiments, the FcRn antagonist is administered at a dose of about 3 mg/kg to about 60 mg/kg. In some embodiments, the FcRn antagonist is administered at a dose of about 10 mg/kg to about 25 mg/kg. In some embodiments, the FcRn antagonist is efgartigimod.

In some embodiments, the FcRn antagonist is administered at a dose of 0.2 mg/kg to 200 mg/kg. In some embodiments, the FcRn antagonist is administered at a dose of about 2 mg/kg to about 200 mg/kg. In some embodiments, the FcRn antagonist is administered at a dose of 2 mg/kg to 120 mg/kg. In some embodiments, the FcRn antagonist is administered at a dose of 3 mg/kg to 60 mg/kg. In some embodiments, the FcRn antagonist is administered at a dose of 10 mg/kg to 25 mg/kg. In some embodiments, the FcRn antagonist is efgartigimod.

In some embodiments, the FcRn antagonist is administered at a dose of about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12.5 mg/kg, about 15 mg/kg, about 17.5 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, or about 200 mg/kg. In some embodiments, the FcRn antagonist is efgartigimod.

In some embodiments, the FcRn antagonist is administered at a dose of 0.2 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, or 200 mg/kg. In some embodiments, the FcRn antagonist is efgartigimod.

In some embodiments, the FcRn antagonist is administered intravenously. In some embodiments, the FcRn antagonist is administered intravenously once weekly, once every two weeks, once every three weeks, once every four weeks, once monthly, or once every six weeks. In some embodiments, the FcRn antagonist is efgartigimod.

In some embodiments, the FcRn antagonist is administered intravenously once weekly or once every two weeks at a dose of about 0.2 mg/kg to about 200 mg/kg. In some embodiments, the FcRn antagonist is administered intravenously once weekly or once every two weeks at a dose of about 2 mg/kg to about 200 mg/kg. In some embodiments, the FcRn antagonist is administered intravenously once weekly or once every two weeks at a dose of about 2 mg/kg to about 120 mg/kg. In some embodiments, the FcRn antagonist is administered intravenously once weekly or once every two weeks at a dose of about 3 mg/kg to about 60 mg/kg. In some embodiments, the FcRn antagonist is administered intravenously once weekly or once every two weeks at a dose of about 10 mg/kg to about 25 mg/kg. In some embodiments, the FcRn antagonist is efgartigimod.

In some embodiments, the FcRn antagonist is administered intravenously once weekly or once every two weeks at a dose of about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12.5 mg/kg, about 15 mg/kg, about 17.5 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, or about 200 mg/kg. In some embodiments, the FcRn antagonist is efgartigimod.

In some embodiments, the FcRn antagonist is administered intravenously once weekly or once every two weeks at a dose of 0.2 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, or 200 mg/kg. In some embodiments, the FcRn antagonist is efgartigimod.

In some embodiments, the FcRn antagonist is administered intravenously once weekly or once every two weeks at a dose of about 10 mg/kg to about 30 mg/kg. In some embodiments, the FcRn antagonist is administered intravenously once weekly or once every two weeks at a dose of about 10 mg/kg to about 25 mg/kg. In some embodiments, the FcRn antagonist is administered intravenously once weekly or once every two weeks at a dose of about 10 mg/kg. In some embodiments, the FcRn antagonist is administered intravenously once weekly or once every two weeks at a dose of about 15 mg/kg. In some embodiments, the FcRn antagonist is administered intravenously once weekly or once every two weeks at a dose of about 20 mg/kg. In some embodiments, the FcRn antagonist is administered intravenously once weekly or once every two weeks at a dose of about 25 mg/kg. In some embodiments, the FcRn antagonist is administered intravenously once weekly or once every two weeks at a dose of about 30 mg/kg. In some embodiments, the FcRn antagonist is administered intravenously once weekly or once every two weeks at a dose of 10 mg/kg to 30 mg/kg. In some embodiments, the FcRn antagonist is administered intravenously once weekly or once every two weeks at a dose of 10 mg/kg to 25 mg/kg. In some embodiments, the FcRn antagonist is administered intravenously once weekly or once every two weeks at a dose of 10 mg/kg. In some embodiments, the FcRn antagonist is administered intravenously once weekly or once every two weeks at a dose of 15 mg/kg. In some embodiments, the FcRn antagonist is administered intravenously once weekly or once every two weeks at a dose of 20 mg/kg. In some embodiments, the FcRn antagonist is administered intravenously once weekly or once every two weeks at a dose of 25 mg/kg. In some embodiments, the FcRn antagonist is administered intravenously once weekly or once every two weeks at a dose of 30 mg/kg. In some embodiments, the FcRn antagonist is efgartigimod.

In some embodiments, the FcRn antagonist is administered subcutaneously. In some embodiments, the FcRn antagonist is administered subcutaneously once weekly, once every two weeks, once every three weeks, once every four weeks, once monthly, or once every six weeks. In some embodiments, the FcRn antagonist is efgartigimod.

In some embodiments, FcRn antagonist is administered subcutaneously at a fixed dose of about 20 mg to about 20,000 mg. In some embodiments, FcRn antagonist is administered subcutaneously at a fixed dose of about 100 mg to about 10,000 mg once weekly, once every two weeks, once every three weeks, once every four weeks, once monthly, or once every six weeks. In some embodiments, the FcRn antagonist is administered subcutaneously at a fixed dose of 750 mg to 3000 mg once weekly, once every two weeks, once every three weeks, once every four weeks, once monthly, or once every six weeks. In some embodiments, the FcRn antagonist is administered subcutaneously at a fixed dose of 1000 mg to 2000 mg once weekly, once every two weeks, once every three weeks, once every four weeks, once monthly, or once every six weeks. In some embodiments, the FcRn antagonist is efgartigimod.

In some embodiments, the FcRn antagonist is administered subcutaneously at a fixed dose of about 20 mg, about 50 mg, about 100 mg, about 250 mg, about 500 mg, about 750 mg, about 1000 mg, about 1500 mg, about 2000 mg, about 3000 mg, about 4000 mg, about 5000 mg, about 6000 mg, about 7000 mg, about 8000 mg, about 9000 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg once weekly, once every two weeks, once every three weeks, once every four weeks, once monthly, or once every six weeks. In some embodiments, the FcRn antagonist is efgartigimod.

In some embodiments, the FcRn antagonist is administered subcutaneously at a fixed dose of 20 mg, 50 mg, 100 mg, 250 mg, 500 mg, 750 mg, 1000 mg, 1500 mg, 2000 mg, 3000 mg, 4000 mg, 5000 mg, 6000 mg, 7000 mg, 8000 mg, 9000 mg, 10,000 mg, 11,000 mg, 12,000 mg, 13,000 mg, 14,000 mg, 15,000 mg, 16,000 mg, 17,000 mg, 18,000 mg, 19,000 mg, or 20,000 mg once weekly, once every two weeks, once every three weeks, once every four weeks, once monthly, or once every six weeks. In some embodiments, the FcRn antagonist is administered subcutaneously at a fixed dose of 1000 mg or 2000 mg once weekly, once every two weeks, once every three weeks, once every four weeks, once monthly, or once every six weeks. In some embodiments, the FcRn antagonist is efgartigimod.

In some embodiments, the FcRn antagonist is administered subcutaneously once weekly or every two weeks at a fixed dose of about 750 mg to about 3000 mg. In some embodiments, the FcRn antagonist is administered subcutaneously once weekly or every two weeks at a fixed dose of about 1000 mg to about 2000 mg. In some embodiments, the FcRn antagonist is administered subcutaneously at a fixed dose of about 1000 mg or about 2000 mg once weekly or every two weeks. In some embodiments, the FcRn antagonist is efgartigimod.

In some embodiments, the FcRn antagonist is administered subcutaneously once weekly or every two weeks at a fixed dose of 750 mg to 3000 mg. In some embodiments, the FcRn antagonist is administered subcutaneously once weekly or every two weeks at a fixed dose of 1000 mg to 2000 mg. In some embodiments, the FcRn antagonist is administered subcutaneously at a fixed dose of 1000 mg or 2000 mg once weekly or every two weeks. In some embodiments, the FcRn antagonist is efgartigimod.

In some embodiments, the FcRn antagonist is first administered subcutaneously at a fixed dose of about 1000 mg twice on the same day. In some embodiments, the FcRn antagonist is first administered subcutaneously at a fixed dose of 1000 mg twice on the same day. In some embodiments, the FcRn antagonist is efgartigimod.

In some embodiments, the FcRn antagonist is administered subcutaneously once weekly at a fixed dose of about 750 mg to about 1750 mg. In some embodiments, the FcRn antagonist is administered subcutaneously once weekly at a fixed dose of about 800 mg to about 1200 mg. In some embodiments, the FcRn antagonist is administered subcutaneously once weekly at a fixed dose of about 750 mg. In some embodiments, the FcRn antagonist is administered subcutaneously once weekly at a fixed dose of about 800 mg. In some embodiments, the FcRn antagonist is administered subcutaneously once weekly at a fixed dose of about 1000 mg. In some embodiments, the FcRn antagonist is administered subcutaneously once weekly at a fixed dose of about 1200 mg. In some embodiments, the FcRn antagonist is administered subcutaneously once weekly at a fixed dose of about 1250 mg. In some embodiments, the FcRn antagonist is administered subcutaneously once weekly at a fixed dose of about 1500 mg. In some embodiments, the FcRn antagonist is administered subcutaneously once weekly at a fixed dose of about 1750 mg. In some embodiments, the FcRn antagonist is efgartigimod.

In some embodiments, the FcRn antagonist is administered subcutaneously once weekly at a fixed dose of 750 mg to 1750 mg. In some embodiments, the FcRn antagonist is administered subcutaneously once weekly at a fixed dose of 800 mg to 1200 mg. In some embodiments, the FcRn antagonist is administered subcutaneously once weekly at a fixed dose of 750 mg. In some embodiments, the FcRn antagonist is administered subcutaneously once weekly at a fixed dose of 800 mg. In some embodiments, the FcRn antagonist is administered subcutaneously once weekly at a fixed dose of 1000 mg. In some embodiments, the FcRn antagonist is administered subcutaneously once weekly at a fixed dose of 1200 mg. In some embodiments, the FcRn antagonist is administered subcutaneously once weekly at a fixed dose of 1250 mg. In some embodiments, the FcRn antagonist is administered subcutaneously once weekly at a fixed dose of 1500 mg. In some embodiments, the FcRn antagonist is administered subcutaneously once weekly at a fixed dose of 1750 mg. In some embodiments, the FcRn antagonist is efgartigimod.

In some embodiments, the FcRn antagonist is administered subcutaneously once weekly at a dose of about 10 mg/kg to about 25 mg/kg. In some embodiments, the FcRn antagonist is administered subcutaneously once weekly at a dose of about 10 mg/kg. In some embodiments, the FcRn antagonist is administered subcutaneously once weekly at a dose of about 15 mg/kg. In some embodiments, the FcRn antagonist is administered subcutaneously once weekly at a dose of about 20 mg/kg. In some embodiments, the FcRn antagonist is administered subcutaneously once weekly at a dose of about 25 mg/kg. In some embodiments, the FcRn antagonist is efgartigimod.

In some embodiments, the FcRn antagonist is administered subcutaneously once weekly at a dose of 10 mg/kg to 25 mg/kg. In some embodiments, the FcRn antagonist is administered subcutaneously once weekly at a dose of 10 mg/kg. In some embodiments, the FcRn antagonist is administered subcutaneously once weekly at a dose of 15 mg/kg. In some embodiments, the FcRn antagonist is administered subcutaneously once weekly at a dose of 20 mg/kg. In some embodiments, the FcRn antagonist is administered subcutaneously once weekly at a dose of 25 mg/kg. In some embodiments, the FcRn antagonist is efgartigimod.

In some embodiments, the FcRn antagonist is first administered intravenously and is subsequently administered subcutaneously. In some embodiments, the FcRn antagonist is first administered intravenously and is subsequently administered subcutaneously at fixed dose of 100 mg to 10,000 mg once weekly, once every two weeks, once every three weeks, once every four weeks, once monthly, or once every six weeks. In some embodiments, the FcRn antagonist is first administered intravenously and is subsequently administered subcutaneously at fixed dose of 1000 mg or 2000 mg once weekly, once every two weeks, once every three weeks, once every four weeks, once monthly, or once every six weeks. In some embodiments, the FcRn antagonist is efgartigimod.

In some embodiments, one or more doses of the FcRn antagonist are administered intravenously and subsequent doses of the FcRn antagonist are administered subcutaneously. In some embodiments, one or more doses of the FcRn antagonist are administered intravenously and subsequent doses of the FcRn antagonist are administered subcutaneously at fixed dose of 100 mg to 10,000 mg once weekly, once every two weeks, once every three weeks, once every four weeks, once monthly, or once every six weeks. In some embodiments, one or more doses of the FcRn antagonist are administered intravenously and subsequent doses of the FcRn antagonist are administered subcutaneously at fixed dose of 1000 mg or 2000 mg once weekly, once every two weeks, once every three weeks, once every four weeks, once monthly, or once every six weeks. In some embodiments, the FcRn antagonist is efgartigimod.

In some embodiments, the FcRn antagonist is administered for 6, 12, 24, 39, or 52 weeks or less. In some embodiments, the FcRn antagonist is administered for 24 weeks or less. In some embodiments, the FcRn antagonist is administered for 52 weeks or less. In some embodiments, the FcRn antagonist is administered for at least 6, 12, 24, 39, or 52 weeks. In some embodiments, the FcRn antagonist is administered for at least 24 weeks. In some embodiments, the FcRn antagonist is administered for at least 52 weeks.

In some embodiments, the FcRn antagonist is rozanolixizumab. In some embodiments, rozanolixizumab is administered subcutaneously or intravenously. In some embodiments, rozanolixizumab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, rozanolixizumab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, the FcRn antagonist is nipocalimab. In some embodiments, nipocalimab is administered subcutaneously or intravenously. In some embodiments, nipocalimab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, nipocalimab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, the FcRn antagonist is orilanolimab. In some embodiments, orilanolimab is administered subcutaneously or intravenously. In some embodiments, orilanolimab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, orilanolimab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered intravenously at a dose of about 30 mg/kg once weekly for three weeks and then at a dose of 10 mg/kg administered intravenously every other week.

In some embodiments, the FcRn antagonist is batoclimab. In some embodiments, batoclimab is administered subcutaneously or intravenously. In some embodiments, batoclimab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, batoclimab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In an embodiment, the subject was previously treated with a corticosteroid and/or an immunosuppressive agent and/or an antimalarial. In an embodiment, the subject was previously treated with prednisone. In an embodiment, the corticosteroid is an oral corticosteroid. Examples of oral corticosteroids include, but are not limited to, betamethasone, cortisone, dexamethasone, hydrocortisone, methylprednisolone, prednisone, prednisolone, and triamcinolone. In an embodiment, the immunosuppressive agent is selected from methotrexate, azathioprine, mycophenolate mofetil, mycophenolic acid, tacrolimus, cyclosporine, leflunomide, or mixoribine. In an embodiment, the antimalarial is selected from hydroxychloroquine, quinacrine, or chloroquine.

In an embodiment, the subject was previously treated with prednisone at a dose of 5 mg/day to 20 mg/day. In an embodiment, the subject was previously treated with prednisone at a dose of 7.5 mg/day to 15 mg/day. In an embodiment, the subject was previously treated with prednisone at a dose of 8 mg/day to 15 mg/day. In an embodiment, the subject was previously treated with prednisone at a dose of 10 mg/day to 15 mg/day.

In an embodiment, the method further comprises administering to the subject an effective amount of a corticosteroid and/or an immunosuppressive agent and/or an antimalarial. In an embodiment, the corticosteroid is an oral corticosteroid. Examples of oral corticosteroids include, but are not limited to, betamethasone, cortisone, dexamethasone, hydrocortisone, methylprednisolone, prednisone, prednisolone, and triamcinolone. In an embodiment, the immunosuppressive agent is selected from methotrexate, azathioprine, mycophenolate mofetil, mycophenolic acid, tacrolimus, cyclosporine, leflunomide, or mixoribine. In an embodiment, the antimalarial is selected from hydroxychloroquine, quinacrine, or chloroquine.

In an embodiment, the effective amount of the corticosteroid is administered at a dose of 2.5 mg/day to 20 mg/day. In an embodiment, the effective amount of the corticosteroid is administered at a dose of 5 mg/day to 20 mg/day. In an embodiment, the effective amount of the corticosteroid is administered at a dose of 7.5 mg/day to 20 mg/day. In an embodiment, the effective amount of the corticosteroid is administered at a dose of 8 mg/day to 15 mg/day. In an embodiment, the effective amount of the corticosteroid is administered at a dose of 10 mg/day to 15 mg/day. In an embodiment, the effective amount of the corticosteroid is administered at a dose of about 20 mg per day. In an embodiment, the effective amount of the corticosteroid is administered at a dose of about 10 mg per day. In an embodiment, the effective amount of the corticosteroid is administered at a dose of about 7.5 mg per day. In an embodiment, the effective amount of the corticosteroid is administered at a dose of about 5 mg/day. In an embodiment, the effective amount of the corticosteroid is administered at a dose of about 2.5 mg/day. In an embodiment, the effective amount of the corticosteroid is administered at a dose of about 5 mg/kg/day. In an embodiment, the effective amount of the corticosteroid is administered at a dose of about 3 mg/kg/day. In an embodiment, the effective amount of the corticosteroid is administered at a dose of about 2 mg/kg/day. In an embodiment, the effective amount of the corticosteroid is administered at a dose of about 1 mg/kg/day. In an embodiment, the effective amount of the corticosteroid is administered at a dose of about 0.5 mg/kg/day. In an embodiment, the effective amount of the corticosteroid is administered at a dose of about 0.4 mg/kg/day. In an embodiment, the effective amount of the corticosteroid is administered at a dose of about 0.3 mg/kg/day. In an embodiment, the effective amount of the corticosteroid is administered at a dose of about 0.25 mg/kg/day. In an embodiment, the effective amount of the corticosteroid is administered at a dose of about 0.2 mg/kg/day. In an embodiment, the effective amount of the corticosteroid is administered at a dose of about 0.1 mg/kg/day.

In an embodiment, the corticosteroid dosing regimen is tapered in the subject after treatment with the FcRn antagonist. In an embodiment, tapering the corticosteroid regimen is lowering the dose or lowering the dosing frequency of the corticosteroid. In an embodiment, the dose of corticosteroid is decreased after 16 weeks of treatment with the FcRn antagonist. In an embodiment, the dose of corticosteroid is decreased after 24 weeks of treatment with the FcRn antagonist. In an embodiment, the dose of corticosteroid is decreased after 16 weeks of treatment with the FcRn antagonist if the dose of corticosteroid is administered to the subject at more than 5 mg/day prior to the dose of corticosteroid being decreased. In an embodiment, the dose of corticosteroid is decreased after 24 weeks of treatment with the FcRn antagonist if the dose of corticosteroid is administered to the subject at more than 5 mg/day prior to the dose of corticosteroid being decreased. In an embodiment, the dose of corticosteroid is decreased after 16 weeks of treatment with the FcRn antagonist if the dose of corticosteroid is administered to the subject at more than 7.5 mg/day prior to the dose of corticosteroid being decreased. In an embodiment, the dose of corticosteroid is decreased after 24 weeks of treatment with the FcRn antagonist if the dose of corticosteroid is administered to the subject at more than 7.5 mg/day prior to the dose of corticosteroid being decreased. In some embodiments, the dose of prednisone is decreased by 2.5 mg/day (or equivalent OCS) after 16 weeks of treatment with the FcRn antagonist. In some embodiments, the dose of prednisone is decreased by 2.5 mg/day (or equivalent OCS) after 24 weeks of treatment with the FcRn antagonist. In some embodiments, each daily dose of prednisone (or equivalent OCS) is maintained for at least two weeks before the dose is further decreased. In some embodiments, each daily dose of prednisone (or equivalent OCS) is maintained for at least three weeks before the dose is further decreased. In some embodiments, each daily dose of prednisone (or equivalent OCS) is maintained for at least four weeks before the dose is further decreased. In some embodiments, each daily dose of prednisone (or equivalent OCS) is maintained for four weeks before the dose is further decreased.

In an embodiment, the first corticosteroid tapering dose may be less than a 2.5-mg dose reduction. In an embodiment, for a baseline dose of 18 to 20 mg, the first decreased dose is 17.5 mg. In an embodiment, for a baseline dose of 16 to 17.5 mg, the first decreased dose is 15 mg. In an embodiment, for a baseline dose of 13 to 15 mg, the first decreased dose is 12.5 mg. In an embodiment, for a baseline dose of 11 to 12.5 mg, the first decreased dose is 10 mg. In some embodiments, for a baseline dose of 8 to 9 mg, the first decreased dose is 7.5 mg. In an embodiment, for a baseline dose of 6 to 7 mg, the first decreased dose is 5 mg. In some embodiments, the dose of corticosteroid is maintained for four weeks before the dose is further decreased. In some embodiments, the dose of corticosteroid is maintained for four weeks and then decreased by 2.5 mg. In some embodiments, each corticosteroid tapering dose is maintained for four weeks and then further decreased by 2.5 mg. In some embodiments, each corticosteroid tapering dose is maintained for four weeks and then further decreased by 2.5 mg until the dose of corticosteroid is ≤5 mg prednisone/day or equivalent. In some embodiments, each corticosteroid tapering dose is maintained for four weeks and then further decreased by 2.5 mg until the dose of corticosteroid is zero (i.e., discontinuation of corticosteroid therapy).

In some embodiments, the corticosteroid is administered at ≤20 mg prednisone/day or equivalent. In some embodiments, the corticosteroid dose is ≤7.5 mg prednisone/day or equivalent. In some embodiments, the corticosteroid dose is ≤5 mg prednisone/day or equivalent.

In some embodiments, the FcRn antagonist is administered in an induction phase and a maintenance phase. In certain embodiments, during the induction phase, the FcRn antagonist is administered once weekly or more frequently, e.g., twice a week or every other day. In certain embodiments, during the induction phase, the FcRn antagonist is administered less frequently than once weekly, e.g., once every other week. In certain embodiments, (i) during the induction phase the FcRn antagonist is administered once weekly concurrently with corticosteroid and/or immunosuppressant and/or antimalarial; and (ii) during the maintenance phase the FcRn antagonist dose is decreased and/or the FcRn antagonist dosing interval is lengthened, e.g., to once every two weeks, with or without concurrent corticosteroid and/or immunosuppressant and/or antimalarial. In some embodiments, the corticosteroid dose is decreased and/or the corticosteroid dosing interval is lengthened during the maintenance phase. In some embodiments, (i) during the induction phase the FcRn antagonist is administered once weekly at a dose of 1000 mg; and (ii) during the maintenance phase the FcRn antagonist is administered once every two weeks at a dose of 1000 mg.

In an embodiment, the subject has a serum level of a pathogenic IgG autoantibody that is associated with myositis (e.g., a myositis-associated antibody (MAA) or a myositis-specific antibody (MSA)). In an embodiment, the pathogenic IgG autoantibody is an anti-HMGCR antibody, an anti-SRP antibody, or an anti-aminoacyl-tRNA synthetase antibody. In an embodiment, the level of a pathogenic IgG autoantibody is measured by ELISA or addressable laser bead immunoassay (ALBIA). In an embodiment, the serum level of a pathogenic IgG autoantibody is compared to baseline levels in the subject.

In an embodiment, the myositis is immune-mediated necrotizing myopathy (EIMNM), dermatomyositis (DM), juvenile dermatomyositis (JDM), polymyositis (PM), or antisynthetase syndrome (ASyS). In some embodiments, the myositis is IMNM.

In some embodiments, treatment of myositis is characterized by the reduction of autoantibodies. In some embodiments, the autoantibodies are anti-SRP autoantibodies. In some embodiments, the prevalence of anti-SRP autoantibodies is reduced by at least 10%, at least 25%, at least 50%, at least 75%, at least 80%, at least 90%, at least 95%, at least 96%, at least 97%, at least 98%, or at least 99% after administering one or more of the treatments described herein. In some embodiments, anti-SRP autoantibodies are undetectable in the subject after administering one or more of the treatments described herein. In some embodiments, the treatment is administration of an effective amount of an FcRn antagonist. In some embodiments, the treatment is administration of an effective amount of efgartigimod. In some embodiments, the myositis is IMNM.

In some embodiments, the autoantibodies are anti-HMGCR autoantibodies. In some embodiments, the prevalence of anti-HMGCR autoantibodies is reduced by at least 10%, at least 25%, at least 50%, at least 75%, at least 80%, at least 90%, at least 95%, at least 96%, at least 97%, at least 98%, or at least 99% after administering one or more of the treatments described herein. In some embodiments, anti-HMGCR autoantibodies are undetectable in the subject after administering one or more of the treatments described herein. In some embodiments, the treatment is administration of an effective amount of an FcRn antagonist. In some embodiments, the treatment is administration of an effective amount of efgartigimod. In some embodiments, the myositis is IMNM.

In some embodiments, treatment of myositis is characterized by total improvement score (TIS). In some embodiments, the TIS is at least 5, at least 10, at least 15, at least 20, at least 25, at least 30, at least 35, at least 40, at least 45, at least 50, at least 55, at least 60, at least 65, or at least 70 points after administering one or more of the treatments described herein. In some embodiments, minimal clinical improvement (TIS ≥20) is observed within ≤24 weeks, ≤12 weeks, or ≤6 weeks after administering one or more of the treatments described herein. In some embodiments, the treatment is administration of an effective amount of an FcRn antagonist. In some embodiments, the treatment is administration of an effective amount of efgartigimod.

In some embodiments, treatment of myositis is characterized by muscle strength improvement. In some embodiments, muscle strength improvement is measured by MMT8. In some embodiments, the MMT8 score is increased by at least 5%, at least 10%, at least 15%, at least 20%, at least 25%, at least 30%, at least 35%, at least 40%, at least 45%, or at least 50% after administering one or more of the treatments described herein. In some embodiments, the treatment is administration of an effective amount of an FcRn antagonist. In some embodiments, the treatment is administration of an effective amount of efgartigimod.

In some embodiments, treatment of myositis is characterized by decrease in patient's global assessment of disease activity (PGA). In some embodiments, the PGA is decreased by at least 5%, at least 10%, at least 15%, at least 20%, at least 25%, at least 30%, at least 35%, at least 40%, at least 45%, at least 50%, at least 55%, or at least 60% after administering one or more of the treatments described herein. In some embodiments, the treatment is administration of an effective amount of an FcRn antagonist. In some embodiments, the treatment is administration of an effective amount of efgartigimod.

In some embodiments, treatment of myositis is characterized by decrease in physician's global assessment of disease activity (MDGA). In some embodiments, the MDGA is decreased by at least 5%, at least 10%, at least 15%, at least 20%, at least 25%, at least 30%, at least 35%, at least 40%, at least 45%, at least 50%, at least 55%, or at least 60% after administering one or more of the treatments described herein. In some embodiments, the treatment is administration of an effective amount of an FcRn antagonist. In some embodiments, the treatment is administration of an effective amount of efgartigimod.

In some embodiments, treatment of myositis is characterized by improvement in extra-muscular global assessment of disease activity. In some embodiments, the extra-muscular global assessment of disease activity is improved by at least 5%, at least 10%, at least 15%, at least 20%, at least 25%, at least 30%, at least 35%, at least 40%, at least 45%, at least 50%, at least 55%, or at least 60% after administering one or more of the treatments described herein. In some embodiments, the treatment is administration of an effective amount of an FcRn antagonist. In some embodiments, the treatment is administration of an effective amount of efgartigimod.

In some embodiments, treatment of myositis is characterized by improvement in pain and/or fatigue reported by a subject. In some embodiments, improvement in pain and/or fatigue in a subject is determined by subject-reported numerical score (NRS). In some embodiments, pain and/or fatigue is improved by at least 5%, at least 10%, at least 15%, at least 20%, at least 25%, at least 30%, at least 35%, at least 40%, at least 45%, at least 50%, at least 55%, or at least 60% after administering one or more of the treatments described herein. In some embodiments, the treatment is administration of an effective amount of an FcRn antagonist. In some embodiments, the treatment is administration of an effective amount of efgartigimod.

In some embodiments, treatment of myositis is characterized by improvement in physical functioning. In some embodiments, improvement in physical functioning is determined by HAQ-DI. In some embodiments, physical functioning is improved by at least 5%, at least 10%, at least 15%, at least 20%, at least 25%, at least 30%, at least 35%, at least 40%, at least 45%, at least 50%, at least 55%, or at least 60% after administering one or more of the treatments described herein. In some embodiments, the treatment is administration of an effective amount of an FcRn antagonist. In some embodiments, the treatment is administration of an effective amount of efgartigimod.

In some embodiments, treatment of myositis is characterized by reduction of muscle necrosis. In some embodiments, muscle necrosis is reduced by at least 5%, at least 10%, at least 15%, at least 20%, at least 25%, at least 30%, at least 35%, at least 40%, at least 45%, at least 50%, at least 55%, or at least 60% after administering one or more of the treatments described herein. In some embodiments, the treatment is administration of an effective amount of an FcRn antagonist. In some embodiments, the treatment is administration of an effective amount of efgartigimod.

In some embodiments, treatment of myositis is characterized by regeneration of muscle fibers. In some embodiments, regeneration of muscle fibers is determined by the presence of centronucleated fibers, wherein an increase in centronucleated fibers indicates regeneration of muscle fibers. In some embodiments, regenerating muscle fibers are increased by at least 5%, at least 10%, at least 15%, at least 20%, at least 25%, at least 30%, at least 35%, at least 40%, at least 45%, at least 50%, at least 55%, or at least 60% after administering one or more of the treatments described herein. In some embodiments, the treatment is administration of an effective amount of an FcRn antagonist. In some embodiments, the treatment is administration of an effective amount of efgartigimod.

In some embodiments, treatment of myositis, in particular dermatomyositis (DM), is characterized by decrease in CDASI. In some embodiments, the CDASI is decreased by at least 5%, at least 10%, at least 15%, at least 20%, at least 25%, at least 30%, at least 35%, at least 40%, at least 45%, at least 50%, at least 55%, or at least 60% after administering one or more of the treatments described herein. In some embodiments, the treatment is administration of an effective amount of an FcRn antagonist. In some embodiments, the treatment is administration of an effective amount of efgartigimod.

In an aspect, provided herein is a method for monitoring treatment of myositis in a subject following treatment with an FcRn antagonist, the method comprising: a) measuring a muscle enzyme, an autoantibody, or both in a blood sample taken from the subject; and b) comparing the muscle enzyme value, autoantibody value, or both to a reference value associated with the autoantibody-mediated disease in the subject, wherein the subject is in remission from the autoantibody-mediated disease if the a muscle enzyme, an autoantibody, or both in the sample is lower than or equal to the reference value.

In an aspect, provided herein is a method for monitoring efficacy of treatment of myositis in a subject following treatment with a first FcRn antagonist, the method comprising: a) measuring in vitro a serum level of a myositis-associated antibody (MAA) or a myositis-specific antibody (MSA) in a blood sample taken from the subject; and b) comparing the serum level of the MAA or the MSA to a reference value associated with myositis in the subject, wherein the treatment is not effective if the serum level of the MAA or the MSA in the sample is greater than or equal to the reference value, and wherein the treatment is effective if the serum level of the MAA or the MSA in the sample is less than the reference value.

In an aspect, provided herein is a method of treating myositis in a subject that has received a first FcRn antagonist and is receiving a corticosteroid dosing regimen, the method comprising: a) administering to the subject an effective amount of a second FcRn antagonist; b) measuring in vitro a serum level of an MAA or an MSA in a blood sample taken from the subject; and c) comparing the serum level of the MAA or the MSA to a reference value associated with myositis in the subject, wherein the corticosteroid dosing regimen is maintained if the serum level of the MAA or the MSA in the sample is greater than or equal to the reference value, or wherein corticosteroid dosing regimen is tapered if the serum level of the MAA or the MSA is less than the reference value.

In an aspect, provided herein is a second FcRn antagonist for use in a method of treating myositis in a subject that has received a first FcRn antagonist and is receiving a corticosteroid dosing regimen, wherein: a) an effective amount of the second FcRn antagonist is administered to the subject; b) a serum level of an MAA or an MSA in a blood sample taken from the subject is measured in vitro; and c) the serum level of the MAA or the MSA is compared to a reference value associated with myositis in the subject, wherein the corticosteroid dosing regimen is maintained if the serum level of the MAA or the MSA in the sample is greater than or equal to the reference value, and wherein corticosteroid dosing regimen is tapered if the serum level of the MAA or the MSA is less than the reference value.

In some embodiments, the corticosteroid dose regimen is tapered to a lower dose amount or a lower dosing frequency. In an embodiment, the method further comprises administering to the subject an effective amount of the second FcRn antagonist if the serum level of the MAA or the MSA in the sample is greater than or equal to the reference value. In an embodiment, the method further comprises administering to the subject an effective amount of a second FcRn antagonist if the serum level of the MAA or the MSA in the sample is greater than or equal to the reference value.

In an aspect, provided herein is a method for treating myositis in a subject comprising: (a) administering to the subject one or more initial doses of an effective amount of a first FcRn antagonist, (b) administering to the subject one or more further doses of an effective amount of a second FcRn antagonist if the serum level of an MAA or an MSA in the subject after step (a) is greater than or equal to a reference value associated with myositis in the subject, or discontinuing treatment with the first FcRn antagonist if the serum level of the MAA or the MSA in the subject after step (a) is less than a reference value associated with active disease in the subject.

In an aspect, provided herein is an FcRn antagonist for use in a method of treating an myositis in a subject, wherein (a) one or more initial doses of an effective amount of a first FcRn antagonist is administered to the subject, and (b) one or more further doses of an effective amount of a second FcRn antagonist is administered to the subject if the serum level of an MAA or an MSA in the subject after step (a) is greater than or equal to a reference value associated with myositis in the subject or the first FcRn antagonist is discontinued if the serum level of the MAA or the MSA in the subject after step (a) is less than a reference value associated with myositis in the subject.

In an embodiment, the effective amount of the first FcRn antagonist is a dose of about 10 mg/kg to about 30 mg/kg, administered intravenously.

In an embodiment, the effective amount of the first FcRn antagonist is a dose of about 750 mg to about 3000 mg, administered subcutaneously.

In an aspect, provided herein is a method for determining if a subject that has previously been treated for myositis using a first FcRn antagonist requires further treatment with a second FcRn antagonist, the method comprising: a) measuring in vitro a serum level of an MAA or an MSA in a blood sample taken from the subject; and b) comparing the serum level of the MAA or the MSA to a reference value associated with myositis in the subject, wherein if the serum level of the MAA or the MSA in the sample is greater than or equal to the reference value, then the subject is need of further treatment with the second FcRn antagonist.

In an aspect, provided herein is a method for treating myositis in a subject comprising: administering to the subject an effective amount of a second FcRn antagonist, wherein the myositis has relapsed in the subject following prior therapy with a first FcRn antagonist and wherein the subject has a serum level of an MAA or an MSA that is greater than or equal to a reference value associated with myositis in the subject.

In an aspect, provided herein is a second FcRn antagonist for use in a method of treating myositis in a subject, wherein the myositis has relapsed in the subject following prior therapy with a first FcRn antagonist and wherein the subject has a serum level of an MAA or an MSA that is greater than or equal to a reference value associated with myositis in the subject.

In an aspect, provided herein is a method for treating myositis in a subject comprising administering to the subject an effective amount of a second FcRn antagonist, wherein the myositis has relapsed in the subject following prior therapy with a first FcRn antagonist, and wherein the effective amount of the FcRn antagonist is determined based on the serum level of an MAA or an MSA in a blood sample taken from the subject.

In an aspect, provided herein is a second FcRn antagonist for use in a method of treating myositis in a subject, wherein the myositis has relapsed in the subject following prior therapy with a first FcRn antagonist, the method comprising administering to the subject an effective amount of a second FcRn antagonist, wherein the effective amount is determined based on the serum level of an MAA or an MSA in a blood sample taken from the subject.

In an aspect, provided herein is a method for monitoring treatment efficacy in a subject following treatment with a first FcRn antagonist, wherein the subject has myositis, the method comprising: a) measuring in vitro a serum level of an MAA or an MSA in a blood sample taken from the subject; and b) comparing the serum level of the MAA or the MSA to a reference value associated with myositis in the subject, wherein the treatment is not effective if the serum level of the MAA or the MSA in the sample is greater than or equal to the reference value, or wherein the treatment is effective if the serum level of the MAA or the MSA is less than the reference value.

In an aspect, provided herein is a method for monitoring remission of myositis in a subject following treatment with a first FcRn antagonist, the method comprising: a) measuring in vitro a serum level of an MAA or an MSA in a blood sample taken from the subject; and b) comparing the serum level of the MAA or the MSA to a reference value associated with myositis in the subject, wherein the subject is in remission from myositis if the serum level of the MAA or the MSA in the sample is lower than or equal to the reference value.

MAAs and MSAs are typically not present in serum of healthy individuals (i.e., an individual not suffering from myositis or other autoantibody-mediated disease). Thus, in an embodiment, the reference value is an undetectable serum level of an MAA or an MSA.

In some embodiments, the reference value is about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the maximum serum level of the MAA or the MSA measured in the subject prior to receiving any treatment for myositis. In an embodiment, the reference value is greater than about 60% of the maximum serum level of the MAA or the MSA measured in the subject prior to receiving any treatment for myositis.

In some embodiments, the reference value is 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%6, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the maximum serum level of the MAA or the MSA measured in the subject prior to receiving any treatment for myositis. In an embodiment, the reference value is greater than 60% of the maximum serum level of the MAA or the MSA measured in the subject prior to receiving any treatment for myositis.

In some embodiments, the reference value is about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the mean serum level of the MAA or the MSA measured in the subject prior to receiving any treatment for myositis. In an embodiment, the reference value is greater than about 60% of the mean serum level of the MAA or the MSA measured in the subject prior to receiving any treatment for myositis.

In an embodiment, the reference value is 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the mean serum level of the MAA or the MSA measured in the subject prior to receiving any treatment for myositis. In an embodiment, the reference value is greater than 60% of the mean serum level of the MAA or the MSA measured in the subject prior to receiving any treatment for myositis.

In an embodiment, the reference value is about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 5⁶%, %57% 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% higher than the lowest serum level of the MAA or the MSA measured in the subject following treatment with the first FcRn antagonist for myositis.

In an embodiment, the reference value is 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% higher than the lowest serum level of the MAA or the MSA measured in the subject following treatment with the first FcRn antagonist for myositis.

In an embodiment, the reference value is about 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, or 6.9-fold more than the lowest serum level of the MAA or the MSA measured in the subject following treatment with the first FcRn antagonist for myositis.

In an embodiment, the reference value is 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, or 6.9-fold more than the lowest serum level of the MAA or the MSA measured in the subject following treatment with the first FcRn antagonist for myositis.

In an embodiment, the subject was previously treated with a first FcRn antagonist at a dose of about 10 mg/kg to about 30 mg/kg, administered intravenously. In an embodiment, the subject was previously treated with a first FcRn antagonist at a dose of about 10 mg/kg, administered intravenously. In an embodiment, the subject was previously treated with a first FcRn antagonist at a dose of about 15 mg/kg, administered intravenously. In an embodiment, the subject was previously treated with a first FcRn antagonist at a dose of about 20 mg/kg, administered intravenously. In an embodiment, the subject was previously treated with a first FcRn antagonist at a dose of about 25 mg/kg, administered intravenously. In an embodiment, the subject was previously treated with a first FcRn antagonist at a dose of about 30 mg/kg, administered intravenously.

In an embodiment, the subject was previously treated with a first FcRn antagonist at a dose of about 10 mg/kg to about 30 mg/kg once weekly, administered intravenously. In an embodiment, the subject was previously treated with a first FcRn antagonist at a dose of about 10 mg/kg once weekly, administered intravenously. In an embodiment, the subject was previously treated with a first FcRn antagonist at a dose of about 15 mg/kg once weekly, administered intravenously. In an embodiment, the subject was previously treated with a first FcRn antagonist at a dose of about 20 mg/kg once weekly, administered intravenously. In an embodiment, the subject was previously treated with a first FcRn antagonist at a dose of about 25 mg/kg once weekly, administered intravenously. In an embodiment, the subject was previously treated with a first FcRn antagonist at a dose of about 30 mg/kg once weekly, administered intravenously.

In an embodiment, the subject was previously treated with a first FcRn antagonist at a dose of about 10 mg/kg to about 30 mg/kg once every two weeks, administered intravenously. In an embodiment, the subject was previously treated with a first FcRn antagonist at a dose of about 10 mg/kg once every two weeks, administered intravenously. In an embodiment, the subject was previously treated with a first FcRn antagonist at a dose of about 15 mg/kg once every two weeks, administered intravenously. In an embodiment, the subject was previously treated with a first FcRn antagonist at a dose of about 20 mg/kg once every two weeks, administered intravenously. In an embodiment, the subject was previously treated with a first FcRn antagonist at a dose of about 25 mg/kg once every two weeks, administered intravenously. In an embodiment, the subject was previously treated with a first FcRn antagonist at a dose of about 30 mg/kg once every two weeks, administered intravenously.

In an embodiment, the subject was previously treated with a first FcRn antagonist at a dose of about 750 mg to about 3000 mg, administered subcutaneously. In an embodiment, the subject was previously treated with a first FcRn antagonist at a dose of about 750 mg, administered subcutaneously. In an embodiment, the subject was previously treated with a first FcRn antagonist at a dose of about 1000 mg, administered subcutaneously. In an embodiment, the subject was previously treated with a first FcRn antagonist at a dose of about 1250 mg, administered subcutaneously. In an embodiment, the subject was previously treated with a first FcRn antagonist at a dose of about 1500 mg, administered subcutaneously. In an embodiment, the subject was previously treated with a first FcRn antagonist at a dose of about 1750 mg, administered subcutaneously.

In an embodiment, the subject was previously treated with a first FcRn antagonist at a dose of about 750 mg to about 3000 mg once weekly, administered subcutaneously. In an embodiment, the subject was previously treated with a first FcRn antagonist at a dose of about 750 mg once weekly, administered subcutaneously. In an embodiment, the subject was previously treated with a first FcRn antagonist at a dose of about 1000 mg once weekly, administered subcutaneously. In an embodiment, the subject was previously treated with a first FcRn antagonist at a dose of about 1250 mg once weekly, administered subcutaneously. In an embodiment, the subject was previously treated with a first FcRn antagonist at a dose of about 1500 mg once weekly, administered subcutaneously. In an embodiment, the subject was previously treated with a first FcRn antagonist at a dose of about 1750 mg once weekly, administered subcutaneously.

In an embodiment, the subject was previously treated with a first FcRn antagonist at a dose of about 750 mg to about 3000 mg once every two weeks, administered subcutaneously. In an embodiment, the subject was previously treated with a first FcRn antagonist at a dose of about 750 mg once every two weeks, administered subcutaneously. In an embodiment, the subject was previously treated with a first FcRn antagonist at a dose of about 1000 mg once every two weeks, administered subcutaneously. In an embodiment, the subject was previously treated with a first FcRn antagonist at a dose of about 1250 mg once every two weeks, administered subcutaneously. In an embodiment, the subject was previously treated with a first FcRn antagonist at a dose of about 1500 mg once every two weeks, administered subcutaneously. In an embodiment, the subject was previously treated with a first FcRn antagonist at a dose of about 1750 mg once every two weeks, administered subcutaneously.

In an embodiment, the subject was also previously treated with a corticosteroid or an immunosuppressive agent. In an embodiment, the subject was previously treated with prednisone.

In an embodiment, the subject was previously treated with prednisone at a dose of 5 mg/day to 20 mg/day. In an embodiment, the subject was previously treated with prednisone at a dose of 7.5 mg/day to 15 mg/day. In an embodiment, the subject was previously treated with prednisone at a dose of 8 mg/day to 15 mg/day. In an embodiment, the subject was previously treated with prednisone at a dose of 10 mg/day to 15 mg/day.

In an embodiment, the subject was previously treated with prednisone at a dose of <5 mg/kg/day. In an embodiment, the subject was previously treated with prednisone at a dose of <3 mg/kg/day. In an embodiment, the subject was previously treated with prednisone at a dose of <2 mg/kg/day. In an embodiment, the subject was previously treated with prednisone at a dose of <1 mg/kg/day. In an embodiment, the subject was previously treated with prednisone at a dose of <0.5 mg/kg/day. In an embodiment, the subject was previously treated with prednisone at a dose of <0.4 mg/kg/day. In an embodiment, the subject was previously treated with prednisone at a dose of <0.3 mg/kg/day. In an embodiment, the subject was previously treated with prednisone at a dose of <0.2 mg/kg/day. In an embodiment, the subject was previously treated with prednisone at a dose of <0.1 mg/kg/day.

In an embodiment, the effective amount of a second FcRn antagonist is a higher dose than the previous treatment with the first FcRn antagonist. In an embodiment, the effective amount of a second FcRn antagonist is a lower dose than the previous treatment with the first FcRn antagonist.

In an embodiment, the effective amount of the second FcRn antagonist is administered more frequently compared to the previous treatment with the first FcRn antagonist. In an embodiment, the effective amount of the second FcRn antagonist is administered less frequently compared to the previous treatment with the first FcRn antagonist.

In an embodiment, the effective amount of the second FcRn antagonist is administered intravenously at a dose of about 10 mg/kg to about 30 mg/kg once weekly, every two weeks, every three weeks, every four weeks, or every six weeks. In an embodiment, the effective amount of the second FcRn antagonist is administered intravenously at a dose of 10 mg/kg once weekly, every two weeks, every three weeks, every four weeks, or every six weeks. In an embodiment, the effective amount of the second FcRn antagonist is administered intravenously at a dose of 15 mg/kg once weekly, every two weeks, every three weeks, every four weeks, or every six weeks. In an embodiment, the effective amount of the second FcRn antagonist is administered intravenously at a dose of 20 mg/kg once weekly, every two weeks, every three weeks, every four weeks, or every six weeks. In an embodiment, the effective amount of the second FcRn antagonist is administered intravenously at a dose of 25 mg/kg once weekly, every two weeks, every three weeks, every four weeks, or every six weeks. In an embodiment, the effective amount of the second FcRn antagonist is administered intravenously at a dose of 30 mg/kg once weekly, every two weeks, every three weeks, every four weeks, or every six weeks.

In an embodiment, the effective amount of the second FcRn antagonist is administered intravenously at a dose of about 10 mg/kg once weekly, every two weeks, every three weeks, every four weeks, or every six weeks. In an embodiment, the effective amount of the second FcRn antagonist is administered intravenously at a dose of about 15 mg/kg once weekly, every two weeks, every three weeks, every four weeks, or every six weeks. In an embodiment, the effective amount of the second FcRn antagonist is administered intravenously at a dose of about 20 mg/kg once weekly, every two weeks, every three weeks, every four weeks, or every six weeks. In an embodiment, the effective amount of the second FcRn antagonist is administered intravenously at a dose of about 25 mg/kg once weekly, every two weeks, every three weeks, every four weeks, or every six weeks. In an embodiment, the effective amount of the second FcRn antagonist is administered intravenously at a dose of about 30 mg/kg once weekly, every two weeks, every three weeks, every four weeks, or every six weeks.

In an embodiment, the effective amount of the second FcRn antagonist is administered subcutaneously at a fixed dose of about 750 mg to about 3000 mg once weekly, every two weeks, every three weeks, every four weeks, or every six weeks. In an embodiment, the effective amount of the FcRn second antagonist is administered subcutaneously at a fixed dose of 1000 mg or 2000 mg once weekly, every two weeks, every three weeks, every four weeks, or every six weeks. In an embodiment, the effective amount of the second FcRn antagonist is administered subcutaneously at a fixed dose of about 750 mg once weekly, every two weeks, every three weeks, every four weeks, or every six weeks. In an embodiment, the effective amount of the second FcRn antagonist is administered subcutaneously at a fixed dose of about 1000 mg once weekly, every two weeks, every three weeks, every four weeks, or every six weeks. In an embodiment, the effective amount of the second FcRn antagonist is administered subcutaneously at a fixed dose of about 1250 mg once weekly, every two weeks, every three weeks, every four weeks, or every six weeks. In an embodiment, the effective amount of the second FcRn antagonist is administered subcutaneously at a fixed dose of about 1500 mg once weekly, every two weeks, every three weeks, every four weeks, or every six weeks. In an embodiment, the effective amount of the second FcRn antagonist is administered subcutaneously at a fixed dose of about 1750 mg once weekly, every two weeks, every three weeks, every four weeks, or every six weeks.

In an embodiment, the effective amount of the second FcRn antagonist is administered subcutaneously at a fixed dose of 750 mg once weekly, every two weeks, every three weeks, every four weeks, or every six weeks. In an embodiment, the effective amount of the second FcRn antagonist is administered subcutaneously at a fixed dose of 1000 mg once weekly, every two weeks, every three weeks, every four weeks, or every six weeks. In an embodiment, the effective amount of the second FcRn antagonist is administered subcutaneously at a fixed dose of 1250 mg once weekly, every two weeks, every three weeks, every four weeks, or every six weeks. In an embodiment, the effective amount of the second FcRn antagonist is administered subcutaneously at a fixed dose of 1500 mg once weekly, every two weeks, every three weeks, every four weeks, or every six weeks. In an embodiment, the effective amount of the second FcRn antagonist is administered subcutaneously at a fixed dose of 1750 mg once weekly, every two weeks, every three weeks, every four weeks, or every six weeks.

In an embodiment, the method further comprises administering to the subject an effective amount of a corticosteroid or an immunosuppressive agent. In an embodiment, the effective amount of the corticosteroid is administered at a dose of about 20 mg/day. In an embodiment, the effective amount of the corticosteroid is administered at a dose of about 15 mg/day. In an embodiment, the effective amount of the corticosteroid is administered at a dose of about 10 mg/day. In an embodiment, the effective amount of the corticosteroid is administered at a dose of about 7.5 mg/day. In an embodiment, the effective amount of the corticosteroid is administered at a dose of about 5 mg/day. In an embodiment, the effective amount of the corticosteroid is administered at a dose of about 4 mg/day. In an embodiment, the effective amount of the corticosteroid is administered at a dose of about 3 mg/day. In an embodiment, the effective amount of the corticosteroid is administered at a dose of about 2 mg/day. In an embodiment, the effective amount of the corticosteroid is administered at a dose of about 1 mg/day. In an embodiment, the effective amount of the corticosteroid is administered at a dose of about 0.5 mg/day.

In an embodiment, the effective amount of the corticosteroid is administered at a dose of about 5 mg/kg/day. In an embodiment, the effective amount of the corticosteroid is administered at a dose of about 3 mg/kg/day. In an embodiment, the effective amount of the corticosteroid is administered at a dose of about 2 mg/kg/day. In an embodiment, the effective amount of the corticosteroid is administered at a dose of about 1 mg/kg/day. In an embodiment, the effective amount of the corticosteroid is administered at a dose of about 0.75 mg/kg/day. In an embodiment, the effective amount of the corticosteroid is administered at a dose of about 0.5 mg/kg/day. In an embodiment, the effective amount of the corticosteroid is administered at a dose of about 0.4 mg/kg/day. In an embodiment, the effective amount of the corticosteroid is administered at a dose of about 0.3 mg/kg/day. In an embodiment, the effective amount of the corticosteroid is administered at a dose of about 0.25 mg/kg/day. In an embodiment, the effective amount of the corticosteroid is administered at a dose of about 0.2 mg/kg/day. In an embodiment, the effective amount of the corticosteroid is administered at a dose of about 0.1 mg/kg/day. In an embodiment, the effective amount of the corticosteroid is administered at a dose of about 0.05 mg/kg/day.

In an embodiment, the subject the corticosteroid dosing regimen is tapered based on the serum level of an MAA or an MSA in a subject with myositis. In an embodiment, tapering the corticosteroid regimen is lowering the dose or lowering the dosing frequency of the corticosteroid. In an embodiment, the tapered corticosteroid dose is ≤5 mg prednisone/day or equivalent. In an embodiment, the tapered corticosteroid dose is <2 mg prednisone/kg/day or equivalent. In an embodiment, the tapered corticosteroid dose is less than or equal to about 1.5, 1.0, 0.75, 0.5, or 0.2 mg prednisone/kg/day or equivalent. In an embodiment, the tapered corticosteroid dose is <0.5 mg prednisone/kg/day or equivalent. In an embodiment, the tapered corticosteroid dose is ≤0.1 mg prednisone/kg/day or equivalent.

In an embodiment, the serum level of the MAA or the MSA is measured by ELISA or addressable laser bead immunoassay (ALBIA).

In an embodiment, the MSA is selected from the group consisting of anti-SRP, anti-HMGCR, anti-Mi-2, anti-TIF1, anti-SAE, anti-NXP, anti-MDA5, anti-Jo-1, anti, PL-7, anti-PL-12, anti-EJ, and anti-OJ.

In an embodiment, the MAA is selected from the group consisting of anti-PM/Scl 75, anti-Ku, anti-snRNP, anti-Ro52 (SSA), anti-Ro/60 (SSA), and anti-La (SSB).

In an embodiment, the first FcRn antagonist and the second FcRn antagonist are each the same FcRn antagonist. In an embodiment, the first FcRn antagonist and the second FcRn antagonist are each a different FcRn antagonist.

In an embodiment, the FcRn antagonist is an Fc region comprising amino acids Y, T, E, K, F, and Y at EU positions 252, 254, 256, 433, 434, and 436, respectively. In an embodiment, the first FcRn antagonist or the second FcRn antagonist is an Fc region comprising amino acids Y, T, E, K, F, and Y at EU positions 252, 254, 256, 433, 434, and 436, respectively.

In an embodiment, the FcRn antagonist is efgartigimod. In an embodiment, the first FcRn antagonist or the second FcRn antagonist is efgartigimod.

In an embodiment, the FcRn antagonist comprises the amino acid sequence of SEQ ID NO: 1, 2, or 3. In an embodiment, the first FcRn antagonist or the second FcRn antagonist comprises the amino acid sequence of SEQ ID NO: 1, 2, or 3.

In an embodiment, the FcRn antagonist is an anti-FcRn antibody. In an embodiment, the first FcRn antagonist is an anti-FcRn antibody. In an embodiment, the second FcRn antagonist is an anti-FcRn antibody.

In an embodiment, the anti-FcRn antibody is rozanolixizumab (UCB7665), nipocalimab (M281), orilanolimab (ALXN1830/SYNT001), or batoclimab (IMVT-1401/RVT1401/IBM9161).

In an embodiment, the first FcRn antagonist is an anti-FcRn antibody and the second FcRn antagonist is efgartigimod. In an embodiment, the first FcRn antagonist is an anti-FcRn antibody and the second FcRn antagonist comprises the amino acid sequence of SEQ ID NO: 1, 2, or 3. In an embodiment, the anti-FcRn antibody is rozanolixizumab (UCB7665), nipocalimab (M281), orilanolimab (ALXN1830/SYNT001), or batoclimab (IMVT-1401/RVT1401/IBM9161). In an embodiment, the patient has not been previously treated with efgartigimod.

In an embodiment, the first FcRn antagonist is selected from the group consisting of rozanolixizumab (UCB7665), nipocalimab (M281), orilanolimab (ALXN1830/SYNT001), or batoclimab (IMVT-1401/RVT1401/HBM9161) and the second FcRn antagonist is an Fc region comprising amino acids Y, T, E, K, F, and Y at EU positions 252, 254, 256, 433, 434, and 436, respectively. In an embodiment, the first FcRn antagonist is selected from the group consisting of rozanolixizumab (UCB7665), nipocalimab (M281), orilanolimab (ALXN1830/SYNT001), or batoclimab (IMVT-1401/RVT1401/HBM9161) and the second FcRn antagonist is efgartigimod.

### EXAMPLES

The following examples are offered by way of illustration, and not by way of limitation.

### Example 1: Development of a Humanized Mouse Model of Immune-Mediated Necrotizing Myopathy (IMNM)

Immune-mediated necrotizing myopathies (IMNM) represent a distinct group of inflammatory myopathies. IMNM are characterized by high creatine kinase levels, and necrosis of skeletal muscle fibers with deposition of C5b-9 membrane attack complex (MAC). Most IMNM patients have autoantibodies (aAbs) directed against signal recognition particle (SRP) or hydroxy-3-methylglutaryl-CoaA reductase (HMGCR). In addition to their role as biomarkers, the relation observed between aAb titers and disease severity has early suggested that these aAb could be pathogenic and therefore the central players in EIMNM pathophysiology.

In vitro experiments show that anti-SRP and anti-HMGCR aAb cause muscle fiber atrophy and impair the fusion of myoblasts, therefore limiting their regenerative capacity. Whereas they are normally intracellular proteins, SRP and HMGCR proteins can be found at the surface of myotubes exposed to purified patient-derived aAb. Passive transfer of IgG from anti-SRP⁺ or anti-HMGCR⁺ IMNM patients to mice induce a muscle deficiency accompanied with IgG and complement deposition on muscle fibers and some level of muscle necrosis. Disease is less pronounced in complement-deficient mice and augmented in mice supplemented with human complement. Together, these results establish the pathogenic role of aAb in IMNM.

Passive transfer of IgG from anti-SRP⁺ or anti-HMGCR⁺ IMNM patients plus human complement to complement C5-deficient mice now represent a useful humanized model of IMNM to evaluate preclinically the efficacy of candidate therapies.

### Example 2: Efficacy Testing of Prophylactic Efgartigimod Treatment in a Humanized Mouse Model of Immune-Mediated Necrotizing Myopathy (IMNM)

Immune-mediated necrotizing myopathy (IMNM) is a severe form of myositis characterized by muscle weakness and elevated creatine kinase levels in serum. On biopsy, necrosis/regeneration of skeletal muscle fibers and deposition of C5b-9 membrane attack complex (MAC) are associated with varying levels of immune cell infiltration. The most frequent autoantibody (aAb) in IMNM patients is directed against hydroxymethylglutaryl-Coenzyme A reductase (HMGCR). Anti-HMGCR aAb are pathogenic and induce disease after adoptive transfer to mice by a mechanism partly involving complement.

Efgartigimod is an IgG1 Fc fragment targeting the neonatal Fc receptor (FcRn). The ability of efgartigimod to prevent muscle weakness and loss of grip strength in the humanized murine model of IMNM described in Example 1 was evaluated.

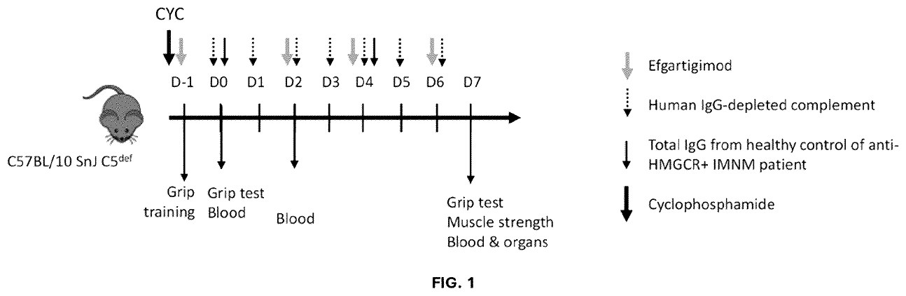

C57BL/10 Snj C5^{def} (C5^{-/-}) mice were treated with total IgG from either an anti-HMGCR-positive IMNM patient or from a healthy donor, as illustrated in the scheme in FIG. 1. Mice (n=8) were dosed with 20 mg/kg efgartigimod by subcutaneous injection (SC), one day before the first injection with 2 mg purified total IgG from an anti-HMGCR-positive IMNM patient. All mice were dosed daily with 100 μL human IgG-depleted complement, as this potentiates the induction of disease by human IMNM autoantibodies anti-HMGCR and anti-SRP (Bergua et al. Ann Rheum Dis, 2019, 78(1): p. 131-139). To prevent xenoimmunization against human proteins, C5^{-/-} mice were pretreated with a single injection of cyclophosphamide (day −1). To demonstrate that grip strength and muscle strength, is reduced by anti-HMGCR, a control group of mice was injected with 2 mg purified total IgG from healthy donors, which should not have an effect on grip strength and muscle strength. Muscle strength was assessed by measurement of gastrocnemius strength upon sciatic nerve electrostimulation (anesthetized animals). Experiments were performed in triplicate.

Total human IgG was measured on day 0, 2 and 7 using the AssayMax™ Human IgG ELISA Kit (Assaypro LLC) according to the manufacturer's protocol.

Anti-HMGCR autoantibody (aAb) titers were measured on day 0, 3, and 7 using an Addressable laser bead immunoassay (ALBIA) was used for the detection and quantification of anti-HMGCR auto antibodies (aAbs) (ALBIA-HMCGR) (Drouot et al., Arthritis Res Ther, 2014, 16(1):p. R39), similar to the ALBIA for anti-SRP antibodies (ALBIA-SRP) described by Benveniste et al. (Arthritis Rheum, 2011, 63(7): p. 1961-71). Briefly, 10 g of recombinant HMGCR C-domain were coupled to 1.25×10⁶ fluorescent Bio-Plex™ COOH-microspheres (Bio-Rad, Hercules, CA, USA) with the Bio-Plex™ amine coupling kit (Bio-Rad) according to manufacturer's protocol. After coupling, coated beads were either used immediately or stored at −20° C. in the dark. When indicated, a human recombinant SRP or intrinsic factor protein (Diarect AG, Freiburg, Germany) was used instead of HMGCR as negative control. Immediately prior to use, HMGCR-coated beads were vigorously agitated for 30 s. Then, a 10 μl volume containing 1,250 beads was added to 100 μl of serum from patients or controls (diluted in Dulbecco's phosphate-buffered saline (DPBS) plus 1% fetal bovine serum) in Multiscreen 96-well plates (Millipore, Bedford, MA, USA). Plates were incubated for 2 h at room temperature in the dark on a plate shaker at 650 rpm. Blank (no serum, secondary antibody only), negative control (anti-HMGCR negative serum) and positive controls (highly positive human anti-HMGCR Ab serum or rabbit anti-HMGCR Abs with appropriate secondary antibody) were included in every assay. Beads were collected by filtration under vacuum and washed twice with 150 μl DPBS containing 0.1% Tween-20. Biotinylated mouse anti-human IgG- or isotype (IgG1, IgG₂, IgG₃ or IgG₄)-specific secondary Ab (Southern Biotech, Birmingham, AL, USA) were added at 1/2,000 dilution and incubated for 1 h at room temperature under shaking. After washing, beads were incubated with 50 μl of streptavidin-R-phycoerythrin (Qiagen, Venlo, The Netherlands) at 1/1,000 dilution for 15 min. Finally, beads were resuspended in 100 μl of DPBS and mean fluorescence intensity (MFI) was determined on a Bio-Plex™ apparatus using the Bio-Plex™ Manager software 4.0 (Bio-Rad). The analytical sensitivity of detection of ALBIA-HMGCR was determined by triplicate dosage of serial dilutions of rabbit anti-HMGCR antibodies. For each well, after subtraction of negative control MFI value, mean MFI and standard deviation (SD) were calculated. The analytical specificity of detection of ALBIA-HMGCR was determined by using high-level human anti-HMGCR, anti-SRP or anti-intrinsic factor positive sera at 1/1,000 dilution. Sera were incubated with purified HMGCR, SRP or intrinsic factor beads for 1 h at room temperature and MFI was determined as described above. Specific inhibition of ALBIA-HMGCR was performed using increasing concentrations of recombinant HMGCR protein, with 1/2,000 dilution of the anti-HMGCR serum used as 100% reference. Percent inhibition was calculated as (1−(MFI pre-adsorbed serum/MFI serum))×100. Homologous and heterologous inhibition of ALBIA-HMGCR was further performed by pre-absorption of three anti-HMGCR, three anti-SRP or three anti-intrinsic factor positive sera with 100 μg/ml of recombinant HMGCR, SRP or intrinsic factor protein. All patients' sera were initially assayed at a 1/500 dilution. The anti-HMGCR Ab levels were determined at a 1/D dilution using the following formula: (MFIserum/MFIcalibrator)×(level of calibrator)×D/500. The calibrator is a human highly positive anti-HMGCR serum (the same throughout the study) whose level was arbitrarily set to 100 arbitrary units (AU)/mL). When sample MFI at 1/500 dilution was higher than 80% of calibrator MFI, further dilutions were performed and the first dilution yielding an MFI inferior to 80% of calibrator MFI was retained for calculation of level.

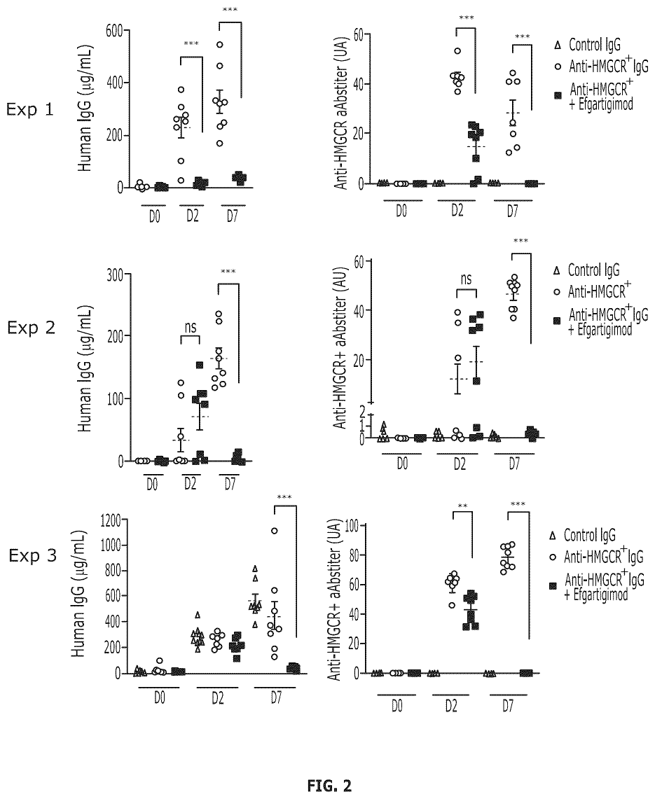

Prophylactic treatment with 20 mg/kg efgartigimod SC significantly reduced the levels of human IgG and anti-HMGCR in recipient mice (FIG. 2). In 2 out of 3 experiments, this reduction was already significant on day 2 after human IgG injection. In all 3 experiments, the reduction of human IgG and anti-HMGCR was significantly reduced on day 7.

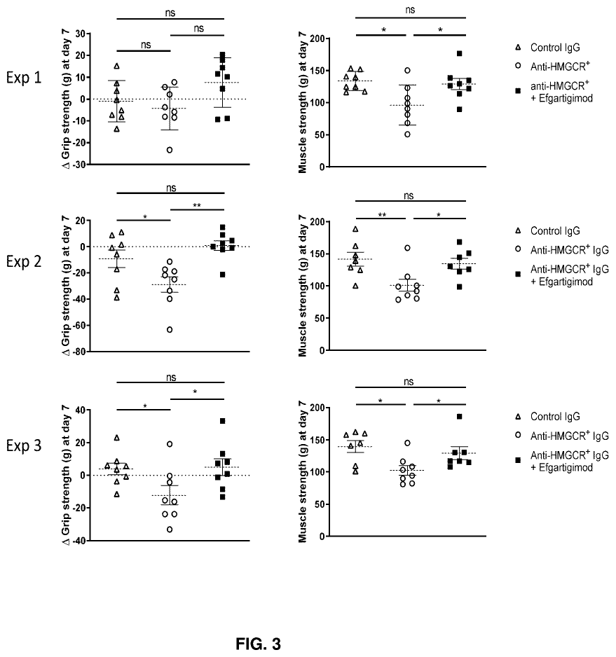

Injection of total IgG from an anti-HMGCR positive patient resulted in a significant loss in grip strength as compared to the mice injected with total IgG from healthy donors (FIG. 3) in 2 out of 3 replicate experiments. In all 3 experiments, injection of total IgG from an anti-HMGCR positive patient resulted in a significant loss in muscle strength. Prophylactic treatment with 20 mg/kg efgartigimod SC prevented anti-HMGCR-induced loss of grip strength and loss of muscle strength.

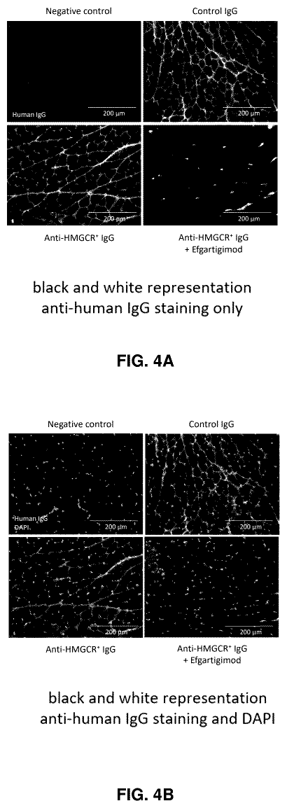

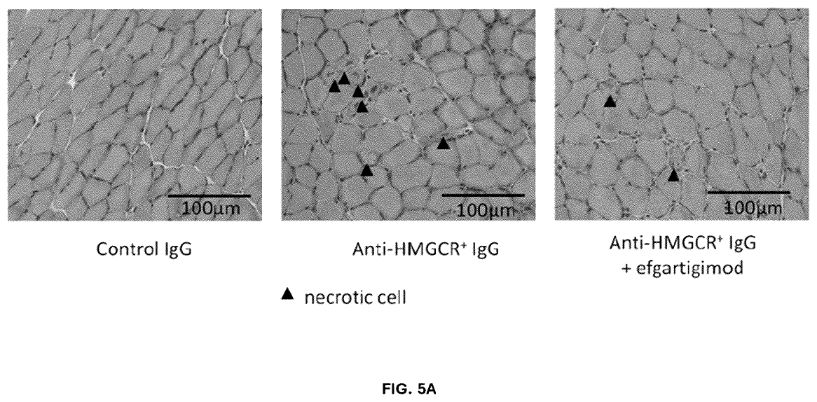

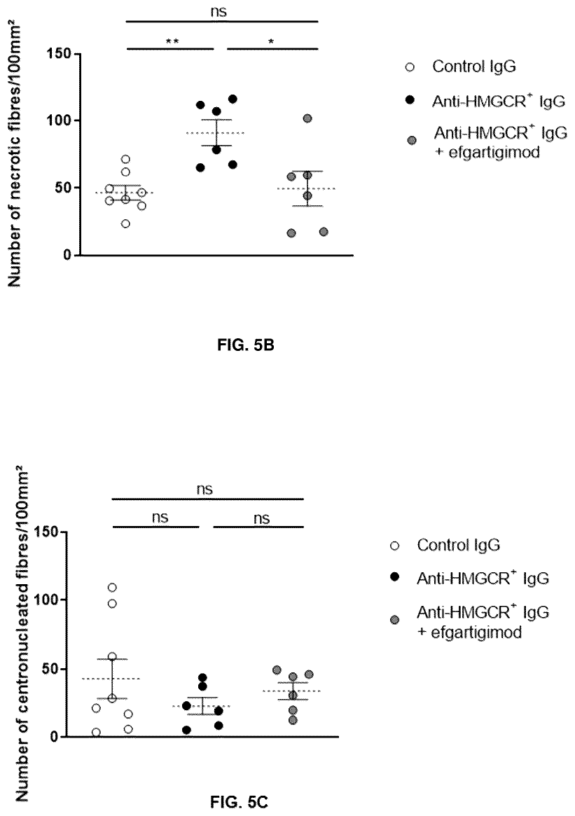

At the end of the in-life phase of the prophylactic experiment the mice were sacrificed and muscle tissue was stained with DAPI (nuclei) and anti-human IgG to stain for human IgG bound to muscle tissue. Mice treated with either total IgG from healthy donors or total IgG from an EIMNM patient positive for anti-HMGCR stained positive for human IgG bound to muscle tissue, while mice treated with efgartigimod and total IgG from an IMNM patient positive for anti-HMGCR stained substantially negative for human IgG (FIGS. 4A-4B). Haematoxylin and Eosin (H&E) staining of cryosections of the gastroenemius muscle isolated from mice treated with total IgG from healthy individuals showed no necrotic cells (FIGS. 5A-5B). Muscle tissue of mice treated with total IgG from anti-HMGCR positive IMNM patients, however, did show necrotic cells (triangles). Necrotic myofibres are defined as pale and/or hyalinized muscle fibres combined with loss of sarcolemmal integrity/coarse appearance. Prophylactic treatment of mice dosed with anti-HMGCR with efgartigimod resulted in less muscle cell necrosis. No regenerating muscle fibers were detected in mice treatment prophylactically with efgartigimod which may be attributed to the time point studied (day 7) being too soon for regeneration to occur.

These experiments demonstrate that injection of patient-derived anti-HMGCR causes loss of grip strength and muscle strength in recipient mice. Muscle weakness is the main symptom patients with IMNM suffer from. Prophylactic treatment with efgartigimod significantly reduced anti-HMGCR levels, and thereby prevented anti-HMGCR-induced loss of grip strength and muscle strength. Furthermore, prophylactic treatment with efgartigimod reduced or prevented myofiber necrosis.

### Example 3: Efficacy Testing of Therapeutic Efgartigimod Treatment in a Humanized Mouse Model of Immune-Mediated Necrotizing Myopathy (IMNM)

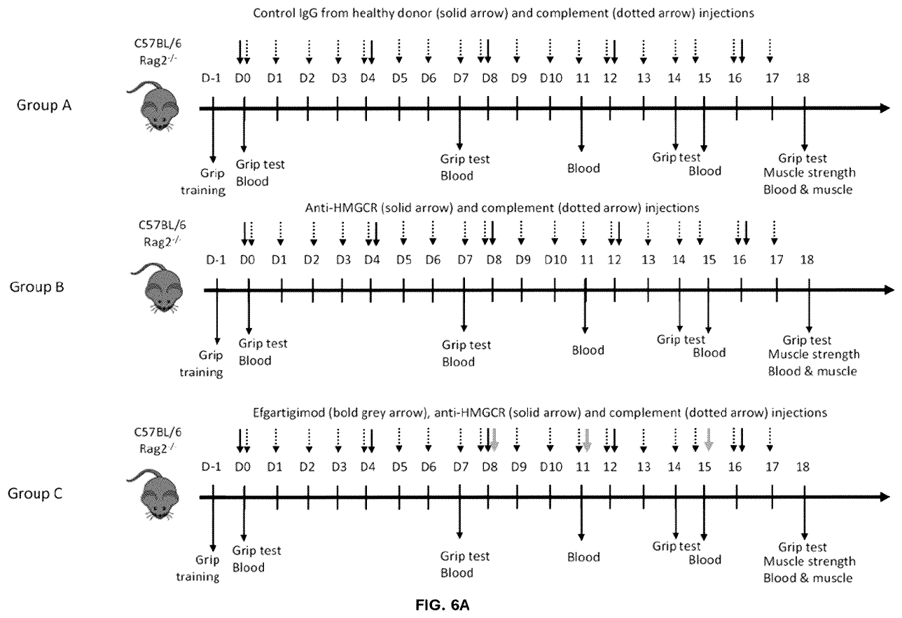

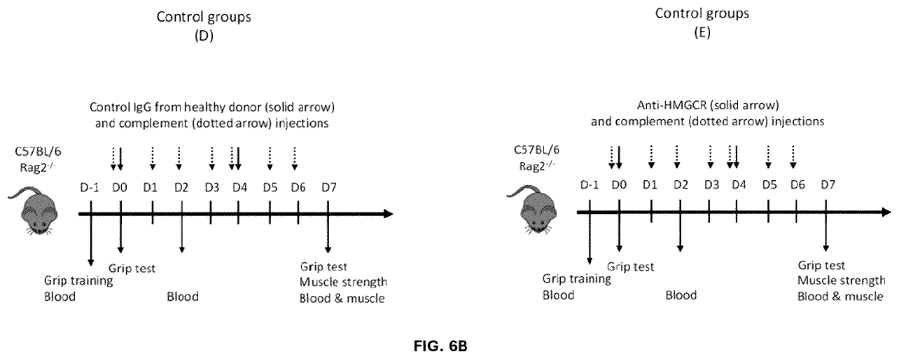

To investigate whether efgartigimod can restore anti-HMGCR-induced muscle weakness and loss of grip strength, C57BL6 Rag2^{-/-} mice were treated with total IgG from either an anti-HMGCR-positive IMNM patient or healthy donor, as illustrated in the schemes in FIGS. 6A-6B. Group A (8 mice) was dosed intraperitoneally (i.p.) with 2 mg control IgG derived from healthy human donors. Group B (8 mice) was dosed i.p. with 2 mg purified total IgG from an anti-HMGCR-positive IMNM patient. Group C was dosed i.p. with 2 mg purified total IgG from an anti-HMGCR-positive IMNM patient. As of day 8, after onset of disease, mice of Group C were treated with efgartigimod 20 mg/kg SC. Control groups D and E were dosed with 2 mg total IgG from either a healthy donor (group D) or from an anti-HMGCR-positive EIMNM patient (group E). All mice were dosed daily with 100 μL human IgG-depleted complement. The rationale for the use of Rag2^{-/-} mice is to prevent xenoimmunization against human proteins.

Muscle strength was measured at day 7 (control groups D and E) and at day 18 (groups A, B and C) by electrostimulation of the open sciatic nerve as described in Example 2. Grip strength was measured at day 7, 14 and 18 as described in Example 2. Total human IgG was measured on day 0, 7, 11 15 and 18 using the AssayMax™ Human IgG ELISA Kit according to the manufacturer's protocol as described in Example 2. Anti-HMGCR titers were measured on day 0, 7, 11, 15 and 18, using the Addressable laser bead immunoassay (ALBIA) described in Example 2(ALBIA-HMCGR).

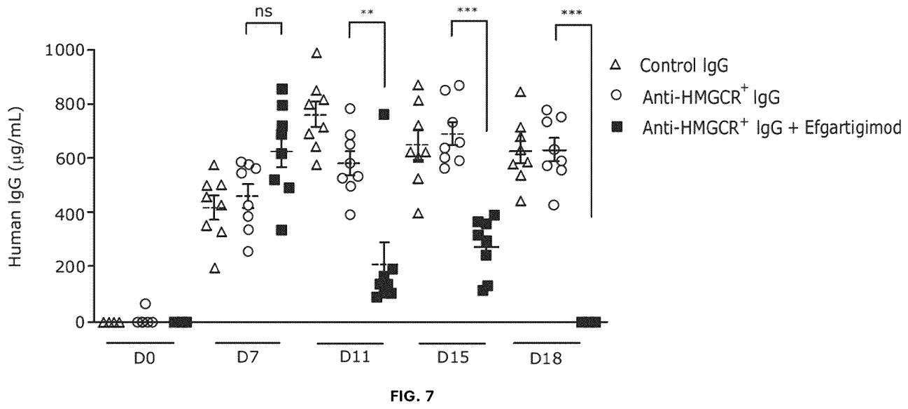

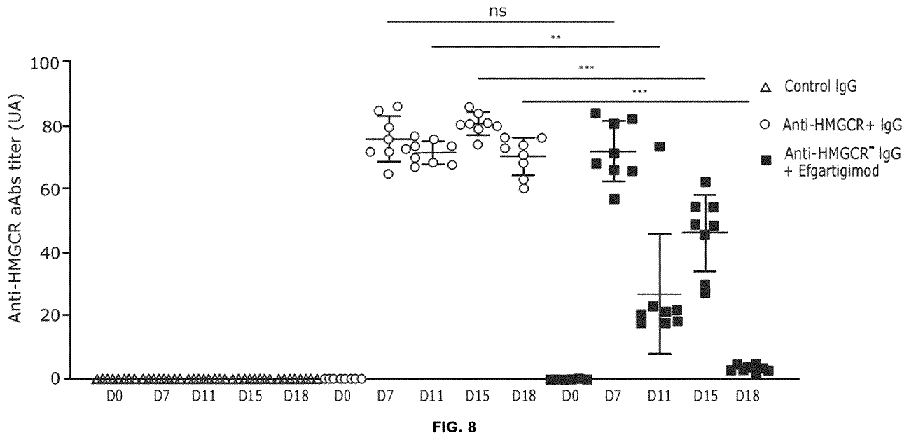

FIG. 7 shows that the injection of 2 mg total human IgG results in clearly measurable titers in the recipient mice. After the first treatment with efgartigimod on day 8, human IgG levels were significantly reduced on day 11, and remained reduced on day 15 and day 18, after new injections of total human IgG on day 12 and day 16, and treatment with efgartigimod on day 11 and day 15. Similarly, treatment with efgartigimod as of day 8 significantly reduced the titer of anti-HMGCR autoantibodies (FIG. 8).

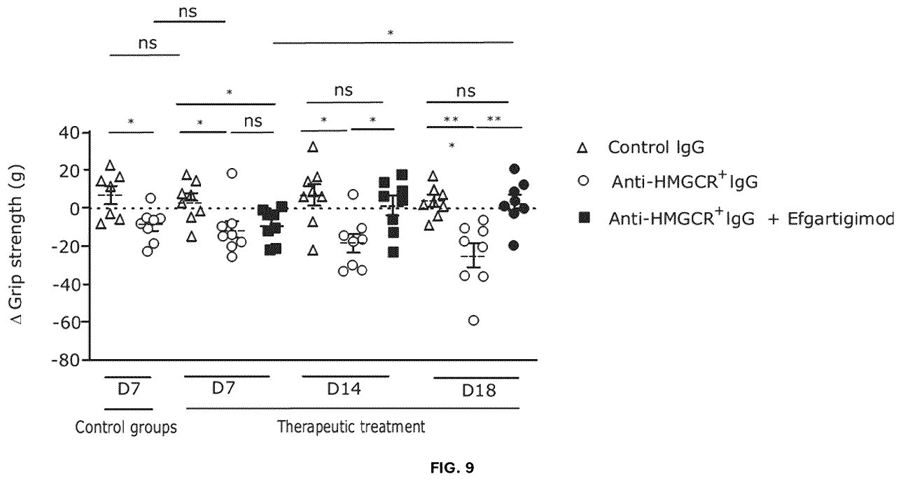

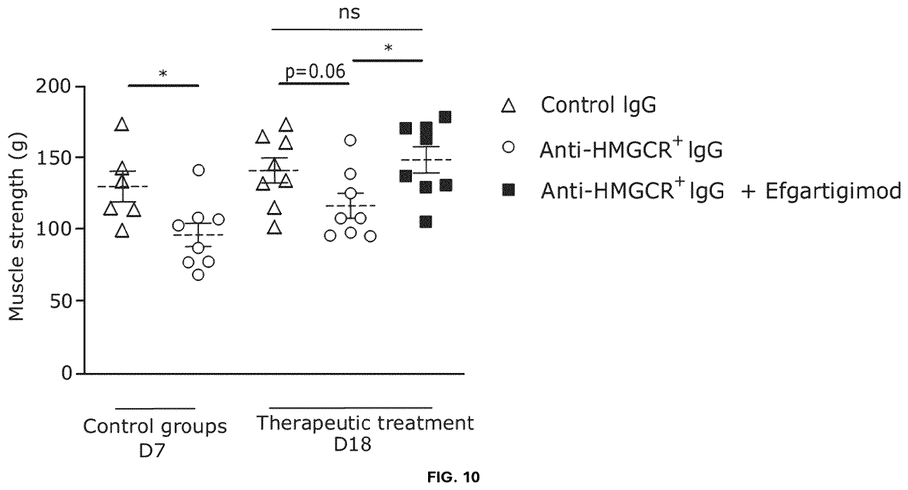

Passive transfer of purified total IgG from an anti-HMGCR positive patient significantly reduced grip strength in recipient mice already as of day 7, whilst mice receiving total IgG from healthy donors retained full grip strength (FIG. 9). Treatment of mice with efgartigimod on day 8, after onset of disease, fully restored grip strength as measured on day 14 and day 18. Similarly, passive transfer of purified total IgG from an anti-HMGCR positive patient resulted in significant loss of muscle strength as measured on day 7. Treatment with efgartigimod as of day 8 resulted in a full recovery of muscle strength on day 18 (FIG. 10).

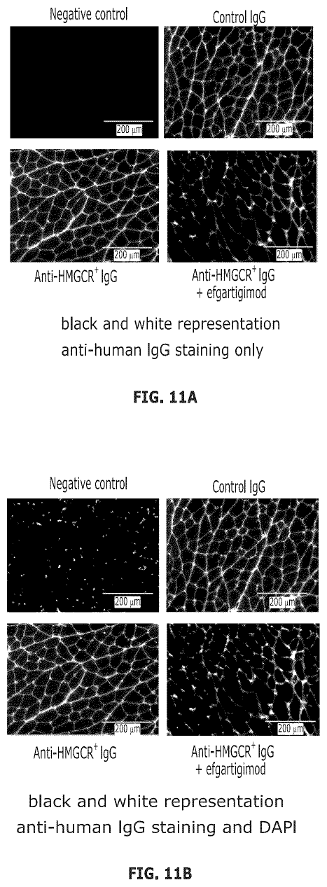

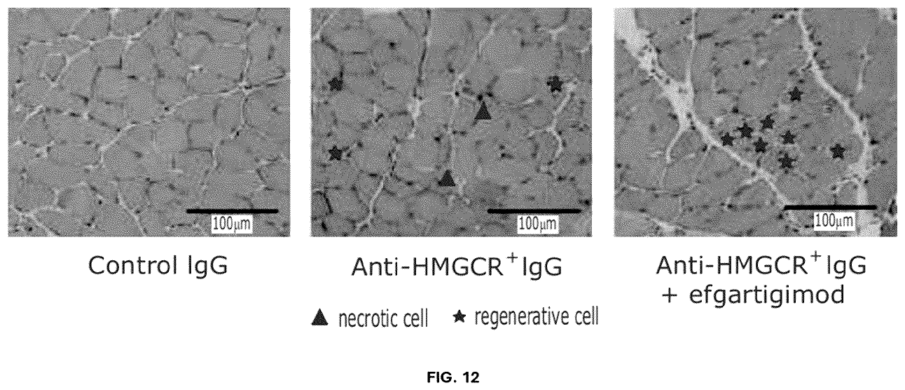

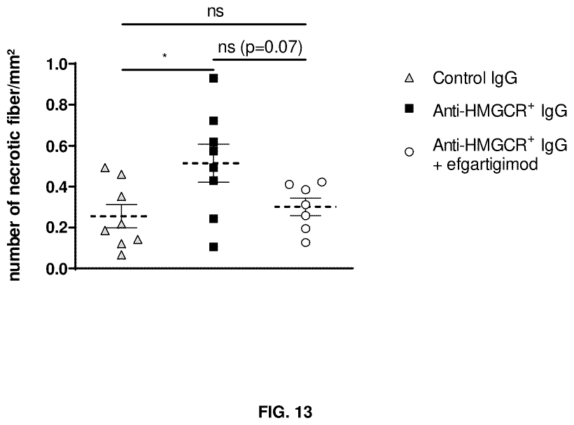

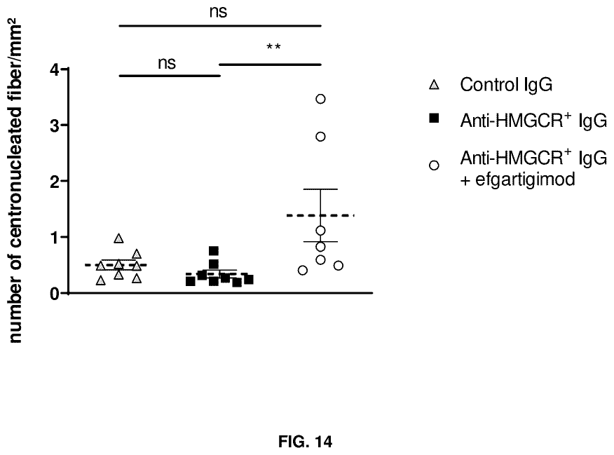

At the end of the in-life phase of the therapeutic experiment the mice were sacrificed and muscle tissue was stained with DAPI (nuclei) and anti-human IgG to stain for human IgG bound to muscle tissue. Mice treated with either total IgG from healthy donors or total IgG from an EIMNM patient positive for anti-HMGCR stained positive for human IgG bound to muscle tissue, while mice treated with efgartigimod and total IgG from an IMNM patient positive for anti-HMGCR stained substantially less positive for human IgG (FIGS. 11A-11B). H&E staining of cryosections of the gastrocnemius muscle tissue isolated from mice treated with total IgG, from healthy individuals, showed no necrotic cells (FIG. 12). Muscle tissue of mice treated with total IgG from anti-HMGCR positive IMNM patients, however, did show necrotic cells (triangles) and regenerating muscle fibers (stars). Regenerating muscle fibers appear as cells with one or more nuclei in the middle of the cell. Muscle tissue isolated from anti-HMGCR dosed mice that were treated as of day 8 with efgartigimod showed less necrotic cells, indicating that treatment with efgartigimod prevents further muscle tissue necrosis in anti-HMGCR-treated mice. Quantification of necrotic cells showed that treatment with efgartigimod results in significantly less necrotic cells as compared to mice dosed with total IgG from an IMNM patient positive for anti-HMGCR that were not treated with efgartigimod (FIG. 13). Muscle tissue isolated from anti-HMGCR dosed mice that were treated as of day 8 with efgartigimod showed substantially more regenerating muscle fibers (stars) as compared to tissue of anti-HMGCR dosed mice that were not treated with efgartigimod. Quantification of regenerating muscle fibers showed that treatment with efgartigimod results in significantly more regenerating muscle fibers as compared to mice dosed with total IgG from an IMNM patient positive for anti-HMGCR that were not treated with efgartigimod (FIG. 14).

In conclusion, therapeutic treatment of mice, in which the grip strength and muscle strength was reduced due to anti-HMGCR injections, results in a full recovery of grip strength and muscle strength. The efficacy of efgartigimod in this model of IMNM is supported by the reduction of pathogenic anti-HMGCR levels, prevention of further muscle fiber necrosis, and regeneration of muscle fibers.

### Example 4: Investigation of Efficacy and Safety of Efgartigimod in Patients with Active Idiopathic Inflammatory Myopathy (IIM)

Idiopathic inflammatory myopathy (IIM) is a heterogeneous group of diseases that includes subtypes with varying pathologies primarily targeting muscle and/or skin and other organs. In many subsets there is a potential role of myositis-specific autoantibodies, most of which are immunoglobulin G (IgG) in the disease pathogenesis. Many patients with IIM have persistent impairment of muscle function, which leads to difficulties in daily life activities and a low health-related quality of life. The typical treatment for IIM is high-dose glucocorticoids combined with immunosuppressive drugs. The deleterious long-term effects of corticosteroids have been well established and include osteoporosis, cataracts, and weight gain. There are no therapies approved by the United States Food and Drug Administration (FDA) or the European regulatory authorities based on results of randomized controlled trials for IMNM and PM. Only 1 licensed treatment (10% intravenous immunoglobulin [IVIg]) is available for adults with DM that was approved based on results of randomized controlled clinical trials.

The neonatal Fc receptor (FcRn) maintains constant levels of IgG in the serum by rescuing IgG antibodies from lysosomal degradation following uptake into cells. Efgartigimod is an engineered Fc fragment that inhibits FcRn function by outcompeting endogenous IgG binding, resulting in reduced IgG recycling and increased IgG degradation. Given efgartigimod's mechanism of action of reducing IgG levels, efgartigimod PH20 SC may benefit patients with these specific IIM subtypes.

ALKIVIA is a global Phase 2/3 randomized, double-blind, placebo-controlled trial of efgartigimod in patients with active IIM. The aim of the study is to investigate the efficacy, safety, tolerability, pharmacodynamics (PD), pharmacokinetics (PK), and immunogenicity of efgartigimod coformulated with recombinant human hyaluronidase PH20 (rHuPH20) administered subcutaneously (SC; referred to throughout as “efgartigimod PH20 SC”) in participants with immune-mediated necrotizing myopathy (IMNM), dermatomyositis (DM), or polymyositis (PM; including PM with antisynthetase syndrome [ASyS]) concomitant to their background treatment for idiopathic inflammatory myopathies (IIM).

To allow for convenient SC injection of investigational medicinal product (IMP), participants will be administered efgartigimod PH20 SC, a solution of efgartigimod coformulated with rHuPH20, or a matching placebo with the same concentration of rHuPH20. rHuPH20 is currently being used in coformulations with approved therapeutic antibodies to facilitate SC injection of volumes >2 mL. Efgartigimod, as both an IV formulation (efgartigimod IV) and efgartigimod PH20 SC, has been investigated in nonclinical studies, clinical studies of healthy volunteers, and clinical studies of patients with IgG-driven autoimmune diseases (generalized myasthenia gravis [gMG], primary immune thrombocytopenia [ITP], chronic inflammatory demyelinating polyneuropathy, and pemphigus).

In clinical studies that included PD assessments, efgartigimod IV and efgartigimod PH20 SC have effectively reduced IgG antibodies. Available clinical data support the clinical benefit of efgartigimod. Efficacy was proven in a pivotal phase 3 study in participants with gMG (ARGX-113-1704), and clear signs of efficacy were demonstrated in phase 2 studies in participants with primary ITP (ARGX-113-1603) or pemphigus (ARGX-113-1701).

The primary objective of ALKIVIA is to evaluate the efficacy and safety of efgartigimod PH20 subcutaneous (SC) treatment compared with placebo, in addition to standard-of-care immunomodulatory therapy, for the treatment of IIM.

### A. Study Design

Overall design

This is a randomized, double-blinded, placebo-controlled, parallel group, multicenter, operationally seamless phase 2/3 study to evaluate the efficacy, safety, tolerability, PK, PD, and immunogenicity of efgartigimod PH20 SC in adult participants with active IIM. This study consists of 2 distinct stages (a phase 2 stage and a phase 3 stage) with separate cohorts of participants.

Participants with the IIM subtypes of IMNM, DM, and PM will be included in the study. The PM subtype includes patients with ASyS as diagnosed under the Connors criteria (i.e., positive serologic testing for anti-tRNA synthetase antibody plus 1 or more of the following clinical features: Raynaud's phenomenon, arthritis, interstitial lung disease, fever that is not attributable to another cause, or evidence of myositis). Participants will be classified into IIM subtypes and meet the disease activity parameters specified.

At screening, participants must be receiving a background IIM treatment of either oral corticosteroids (OCS), 1 antimalarial, or 1 protocol-specified immunosuppressant. Participants may receive a combination of either OCS and 1 antimalarial or OCS and 1 immunosuppressant. Background treatment for IIM has a required duration of treatment at a stable dose. The screening period is up to 4 weeks, but an ad hoc extension may be permitted in certain circumstances.

All screening evaluations must be completed and reviewed to confirm that potential participants meet all eligibility criteria. After the assessments are complete, laboratory results are reported, and, if applicable, eligibility is adjudicated, then the participant may be randomized. Certain specified screening assessments will be submitted for medical monitor (MM) review before the investigator's decision to randomize a participant. The sponsor will also review specified screening assessments for a subset of participants.

An independent data monitoring committee (IDMC) is appointed for this study. In addition, an independent committee comprising members with expertise in IIM is appointed to review historical biopsy data and adjudicate the eligibility of participants with a diagnosis of PM or IN1M who had a negative MSA test result (central laboratory) or PM-Sjögren syndrome overlap (regardless of MSA result).

After screening, a 24-week (phase 2 stage) or 52-week (phase 3 stage) treatment period follows, during which participants will be randomized to receive either efgartigimod PH20 SC 1000 mg or matching placebo (with the same concentration of rHuPH20) weekly in addition to their background treatment for IIM. Background treatment must follow protocol guidelines during the treatment period.

In both stages, during the treatment period, participants will receive weekly SC fixed doses of IMP (either efgartigimod PH20 SC or matching placebo) except in the last week (week 24 [phase 2 stage] or week 52 [phase 3 stage]) in which participants will attend the final treatment period visit. At the end of the treatment period (i.e., week 24 [visit 7, phase 2 stage] or week 52 [visit 14, phase 3 stage]), eligible participants may enroll in the open-label extension (OLE) study ARGX-113-2011, in which all participants will be treated with efgartigimod PH20 SC. Otherwise, participants will continue a 56-day safety follow-up period.

During the treatment period, IMP will be administered at the investigative site at scheduled visits and may be performed at the site or at the participant's home, either by a home nurse or by fully trained participants/caregivers.

### Phase 2 Stage

Approximately 90 adult participants will be enrolled and randomized in the phase 2 stage of the study, stratified by (1) clinical IIM subtype (IMNM, DM, or PM) and (2) MDGA (severe [MDGA ≥5] or non-severe [MDGA <5]). Participants will be randomized in a 1:1 ratio within each stratum to receive either efgartigimod PH20 SC or matching placebo, concomitant to their background treatment for IIM. Randomization of approximately 30 participants of each JIM subtype is targeted. The number of MSA-seronegative participants will be capped at 30% of all participants.

Final data from the phase 2 stage will be analyzed to assess proof of concept in each disease subtype and to support decisions for a predictive enrichment, to stop the study for futility if there is a lack of efficacy in all disease subtypes, and to confirm the design of the phase 3 stage. The unblinded phase 2 stage data will be analyzed and used to recommend adaptation of the design of the phase 3 stage. Only the phase 2 stage data will be unblinded; the sponsor and all clinical staff will remain blinded to all phase 3 stage data until the study is completed.

Phase 3 stage

Recruitment for the phase 3 stage of the study will commence after 90 participants have been randomized in the phase 2 stage. The two stages of this study will run in parallel during the time between commencing of recruitment for the phase 3 stage and the last participant visit in the phase 2 stage.

The design and sample size for the phase 3 stage will be confirmed after analysis of the phase 2 stage data. It is planned that approximately 150 adult participants will be enrolled and randomized in the phase 3 stage, stratified by (1) clinical IIM subtype (IMNM, DM, or PM) and (2) MDGA (severe [MDGA ≥5] or nonsevere [MDGA <5]). Participants will be randomized in a 1:1 ratio within each stratum to receive either efgartigimod PH20 SC or placebo, concomitant to their background treatment for IIM. The number of MSA-seronegative participants will be capped at 30% of all participants.

For any participants in the phase 3 stage of the study with a deselected IIM subtype based on data from the phase 2 stage, the investigator and the participants will be informed. Participants will permanently discontinue the study and complete the EDV and safety follow-up visit. However, if the participant is benefiting from IMP and the investigator determines that continuing treatment with the IMP is in the participant's best interest, they will have the option to discontinue from this study, complete the EDV, and enroll in ARGX-113-2011.

### Rationale for Primary Endpoint

The primary endpoint of the study is the TIS (as assessed by the 2016 American College of Rheumatology [ACR]/European League Against Rheumatism [EULAR] criteria) in the placebo and efgartigimod groups. This score is the weighted sum of improvement in 6 core set measures (CSMs) for disease activity: MDGA, PGA, MMT8, HAQ-DI, muscle enzymes, and extramuscular disease activity.

This composition of measures is designed to allow demonstration of efficacy in DM and PM (of which IMNM is a subset). To facilitate combined clinical studies, myositis experts used a common response criterion for adult DM/PM, acknowledging the limitation that the ACR/EULAR response criteria were developed using a PM diagnosis based on Bohan and Peter's classification criteria, in which PM may include different syndromes, such as IMNM. These response criteria will still be applicable to these newer entities given that the data driven and consensus-driven processes used herein were inclusive of those syndromes.

The response criteria have been endorsed by the ACR and EULAR. The TIS comprises CSMs scored both by the patient and the physician, considering the muscle manifestations and the extramuscular disease activity and physical function. It also includes objective disease activity parameters, i.e., the activity of the muscle enzymes. The International Myositis Assessment and Clinical Studies Group (IMACS) recommends the minimal TIS as the primary endpoint in IIM therapeutic studies. The criteria may be used as a continuous outcome measure, using the TIS, or as a categorical outcome of improvement (minimal, moderate, or major improvement). IMACS considers the threshold of minimal TIS to differentiate between treatment groups in clinical studies as clinically significant. IMACS has previously developed and partially validated International Consensus on Preliminary Definitions of Improvement in Adult and Juvenile Myositis, which combined CSMs of disease activity and defined a minimal degree of clinically meaningful change in each of the CSMs. These criteria were published and previously used as endpoints in myositis therapeutic studies. With the development of the TIS, these criteria/definitions of improvement are no longer recommended for use. To date, several phase 3 studies in patients with IIM have used the TIS as a primary endpoint (NCT02728752, NCT03981744, NCT04044690, and NCT04999020).

Response rates at the end of the treatment period, time to response, and response duration in the TIS (secondary endpoints) will provide supporting evidence for the primary endpoint.

### Rationale for Inclusion Criteria Cutoffs

The inclusion threshold cutoffs for MDGA, PGA, MMT8, and CDASI are based on previous clinical trials and input from key opinion leaders over the past 10 to 15 years.

For MDGA and PGA, the minimum threshold of 2/10 or more is based on the “Rituximab in Myositis” study, which was the largest clinical study in myositis funded by the National Institutes of Health (NIH). These thresholds were based on discussions among the key opinion leaders and underwent a peer review process for the NIH grant. More importantly, these thresholds have been validated by multiple phase 2 and 3 clinical studies in participants with IIM, including the ProDERM study, the Acthar study, the Rituximab in Myositis study, and the tocilizumab clinical study (NCT02043548).

The MMT8 threshold of 142/150 is based on the ProDERM study and key opinion leader discussions. An MMT8 of <142/150 is identified as significant weakness that leads to difficulty in activities of daily living and affects quality of life. Moreover, a threshold of 142 gives approximate mean MMT8 of 125, which is severe weakness in patients with IIM.

For eligibility of participants with DM, the CDASI threshold for the phase 2 stage has been set to ≥7, which is established as the mean for mild skin disease activity. The CDASI threshold for the phase 3 stage has been set to >14, which is the cutoff for mild to moderate skin disease activity.

The 30% cap on MSA-seronegative participants is based on a review of real-world data and corresponds to estimates of MSA prevalence in patients with DM or PM.

### Rationale for Treatment Periods

The selected 24-week treatment period during the phase 2 stage follows the International Council for Harmonisation (ICH) E1 guideline for the safety evaluation of drugs intended for the long-term treatment (chronic or repeated intermittent use for longer than 6 months) of non-life threatening diseases. This guideline states that the number of patients treated for 6 months at dosage levels intended for clinical use should be adequate to characterize the pattern of adverse drug effects over time.

The selected 52-week treatment period during the phase 3 stage accounts for the different JIM subtypes that are slowly progressive and chronic with a waxing and waning disease course. After confirming treatment effect in the selected disease subtypes, this longer treatment period will more thoroughly assess the durability of treatment effect and permit full assessment of the primary and key secondary endpoints.

### Justification for Dose

The sponsor has selected a dose of 1000 mg efgartigimod with rHuPH20 to be administered as weekly SC injections over the full duration of the study as the appropriate dosage regimen for further clinical development.

Initially, the clinical development of efgartigimod was based on IV dosing. Results from phase 1 studies in healthy subjects, phase 2 and 3 studies in patients with gMG, a phase 2 study in patients with primary ITP, a phase 2 study in patients with pemphigus, and a PK/PD modeling analysis showed that efgartigimod IV 10 mg/kg administered once weekly achieved nearly maximal reduction in serum IgG levels. In interventional studies of patients with gMG or ITP, this dose regimen resulted in clinical efficacy outcomes and reduction in pathogenic autoantibody levels while also maintaining a favorable safety and tolerability profile.

The SC route of administration offers convenience for patients with IIM, their caregivers, and health care providers, because SC injections are easier to administer than IV injections. Additionally, the coformulation of efgartigimod with rHuPH20 permits SC dosing of higher volumes than typical SC injections, because rHuPH20 reduces resistance to fluid flow and increases dispersion and absorption of injected medicines and fluids, which allows for a larger volume to be injected with limited skin swelling or pain.

In this phase 2/3 study, participants will be administered efgartigimod PH20 SC as a flat dose (rather than an adjusted dose based on body weight changes), because this is more convenient for study participants and site staff and may lower the risk of dosing errors. Results from a PK/PD modeling analysis indicate that efgartigimod PH20 SC 1000 mg once weekly would result in serum IgG levels equivalent to a once-weekly dose of efgartigimod IV 10 mg/kg. Results from a phase 1 study comparing the PK and PD properties of IV and SC administrations of efgartigimod in healthy subjects (ARGX-113-1907) confirmed that 4 once-weekly injections of efgartigimod PH20 SC 1000 mg was noninferior to 4 once-weekly infusions of efgartigimod IV 10 mg/kg in reducing serum IgG levels. Therefore, the sponsor has selected efgartigimod PH20 SC 1000 mg once weekly as the dose regimen for this study.

The dosage regimen of weekly dose administration throughout the study was selected to achieve and maintain the maximal PD effect: a steady-state reduction of IgGs, including pathogenic autoantibodies. This dose regimen is anticipated to maximize the chance of inducing a fast clinical improvement and to maintain this improvement during the 24-week study period. Based on the chronic dosage data from nonclinical studies and the completed and ongoing clinical studies with efgartigimod PH20 SC, it is anticipated that this chronic dosage regimen in IIM will be well tolerated.

### B. Study Population

Prospective approval of protocol deviations to recruitment and enrollment criteria, also known as protocol waivers or exemptions, is not permitted.

Certain specified screening assessments will be submitted for sponsor review before the investigator's decision to randomize a participant.

In addition, the eligibility of those participants with a clinical diagnosis of PM or IMNM who had a negative MSA test result (central laboratory) or PM Sjögren overlap (regardless of their MSA test result) will be adjudicated by an independent committee selected for their expertise in IIM.

### Inclusion Criteria

Participants are eligible to be included in the study only if all of the following criteria apply:
Aged at least 18 years (or the legal age of consent in the jurisdiction in which the study is taking place) at the time of signing the informed consent;
A definite or probable clinical diagnosis of IIM under the 2017 EULAR/ACR classification criteria;
One of the following medical histories:
Diagnosis of DM or juvenile DM (JDM), fulfilling the 2017 EULAR/ACR criteria for DM or JDM (age of disease onset <18 years of age). The diagnosis date for JDM should not be >5 years from the screening date; Diagnosis of PM (including ASyS), having either of the following: 1) a prior muscle biopsy diagnostic for IIM; 2) a positive test at the central laboratory for at least 1 anti-aminoacyl-tRNA synthetase MSA (-Jo-1, -PL-7, -PL-12, -EJ, -OJ);
Diagnosis of IMNM, meeting the 2017 European Neuromuscular Center classification criteria, and having any of the following: 1) anti-signal recognition particle (SRP) positive (at the central laboratory) myopathy; 2) anti-3-hydroxy-3-methylglutaryl coenzyme A reductase (HMGCR) positive (at the central laboratory) myopathy; 3) a prior muscle biopsy with pathological features of EINM.
Participants with PM overlap with Sjögren's syndrome must meet the ACR/EULAR 2016 classification criteria for Sjögren's syndrome and will be included only if the muscle biopsy does not demonstrate any of the following: P62 (+) inclusions, rimmed vacuoles, or increased number of ragged red fibers and cytochrome oxidase fibers for the patient's age.
Note: Participants with a clinical diagnosis of PM who have a negative MSA result and participants with a clinical diagnosis of PM Sjögren's overlap regardless of their MSA result will be included after adjudication by an independent committee selected for their expertise in IIM.

Note: Participants with a clinical diagnosis of IMNM who have negative anti-SRP and anti-HMGCR results will be included after adjudication by an independent committee selected for their expertise in IIM.
Active disease as defined by the presence of at least 1 of the following criteria: Abnormal levels of at least 1 of the following enzymes: CK (≥4×upper limit of normal [ULN]), aldolase (≥4×ULN), lactate dehydrogenase (LDH) (≥4×ULN), AST (≥4×ULN), or ALT (≥4×ULN), based on central laboratory results;
Electromyography demonstrating active disease within the past 3 months;
Active DM skin rash (Gottron's papules, Gottron's signs, or heliotrope rash) or CDASI ≥7 (phase 2 stage) or ≥14 (phase 3 stage) at screening and baseline;
Muscle biopsy indicative of active JIM in the past 3 months;
Magnetic resonance imaging within the past 3 months indicative of active inflammation.
Muscle weakness as assessed by an MMT8 score of ≤142/150 and abnormalities in any 2 of the other 5 CSMs: 1) MDGA ≥2; 2) PGA ≥2; 3) Extramuscular global ≥2; 4) HAQ-DI ≥0.25; 5) Muscle enzyme ≥1.5×ULN.
Receiving a permitted background treatment for JIM. Permitted background treatment includes: OCS; 1 antimalarial (hydroxychloroquine, quinacrine, or chloroquine); or 1 of the following immunosuppressants: methotrexate, azathioprine, mycophenolate mofetil, mycophenolic acid, tacrolimus, cyclosporine, leflunomide, or mizoribine. Participants may receive a combination of either OCS and up to 1 antimalarial or OCS and up to 1 immunosuppressant.
Permitted background treatment must meet the following dose duration(s):
1) Immunosuppressants or antimalarials administered for at least 12 weeks before screening, and at a stable dose for at least 8 weeks before screening and then through baseline. Participants who stopped treatment with an immunosuppressant or an antimalarial are eligible if their last dose was >8 weeks before screening (except for leflunomide). Participants who stopped treatment with leflunomide are eligible if their last dose was >12 weeks before screening unless leflunomide washout treatment was used, in which case participants are eligible if the washout treatment was completed >4 weeks before screening. Note: The MM should be contacted if a participant is receiving Chinese traditional medicine to discuss any possible immunosuppressive properties thereof. 2) OCS (≤20 mg prednisone or dose equivalent) initiated >8 weeks before screening and at a stable dose for ≥4 weeks before screening through baseline. Participants who stopped treatment with OCS before enrollment are eligible if the last dose of the steroid was >4 weeks before screening. Concurrent stable (>2 weeks before screening) use of topical corticosteroid (or other immunomodulator) therapy is permitted if used for a non-IIM related condition.
Note: If a participant is receiving both OCS and an additional agent, the dose durations described for both (1) and (2) must be met.
Contraceptive use by nonsterilized male participants and women of childbearing potential (WOCBP) will be consistent with local regulations, where available, for individuals participating in clinical studies. WOCBP must have a negative serum pregnancy test during screening and a negative urine pregnancy test at baseline to establish the nonpregnant state before receiving IMP.
Capable of giving signed informed consent, which includes compliance with the requirements and restrictions listed in the informed consent form (ICF) and this protocol.

### Exclusion Criteria

Participants are excluded from the study if the following criteria apply:
A clinically significant uncontrolled active or chronic bacterial, viral, or fungal infection at screening.
A COVID-19 polymerase chain reaction (PCR)-positive test <72 hours before enrollment. COVID-19 testing will be done only if the participant is symptomatic or required by applicable law. In these cases, a negative PCR test (central or local laboratory) is required within 72 hours before enrollment and should occur regardless of a participant's vaccination status.
Any other known autoimmune disease that, in the investigator's opinion, would interfere with an accurate assessment of clinical symptoms of IIM or put the patient at undue risk.
A history of malignancy unless considered cured by adequate treatment, with no evidence of recurrence for ≥3 years before the first administration of IMP. Adequately treated participants with the following cancers can be included at any time: 1) Basal cell or squamous cell skin cancer; 2) Carcinoma in situ of the cervix; 3) Carcinoma in situ of the breast; 4) Incidental histological finding of prostate cancer (TNM stage T1a or T1b). Note: Because patients with IIM have an increased risk of an associated malignancy, it is recommended that participants in the study comply with and are current with local recommendations for cancer screening procedures (based on their age, gender, and autoantibodies).
Severe muscle damage defined as a global muscle damage score of >5 on a 10-cm visual analog scale (VAS) scale on the Myositis Damage Index (MDI).
Glucocorticoid-induced myopathy that the investigator considers the primary cause of muscle weakness or permanent weakness linked to a non-IIM cause.
JDM diagnosed >5 years from screening or JDM with extensive calcinosis (defined as calcinosis involving the torso or 2 extremities) or severe calcinosis (indicated by a calcinosis associated with severe loss of function).
Uncontrolled interstitial lung disease or any other uncontrolled IIM manifestation that, in the opinion of the investigator, would be likely to require treatment with prohibited medication (e.g., cyclophosphamide) during the study.
Other inflammatory and noninflammatory myopathies: inclusion body myositis (based on the biopsy or when the weakness affects the finger and/or the wrist flexors out of proportion to shoulder abductors), overlap myositis (connective tissue disease-associated myositis, except an overlap with Sjögren's syndrome, which is allowed provided that the biopsy does not demonstrate any of the features listed in inclusion criterion 3b), metabolic myopathies, muscle dystrophies or a family history of muscle dystrophy, drug induced or endocrine induced myositis (except statin-induced IMNM), and juvenile myositis (other than JDM).
Clinically significant disease, recent major surgery (within 3 months of screening) or intends to have surgery during the study, or has any other condition in the opinion of the investigator that could confound the results of the study or put the patient at undue risk.
Known hypersensitivity reaction to IMP or 1 of its excipients.
Received a live or live-attenuated vaccine less than 4 weeks before screening. The receipt of other types of vaccines at any time before screening is not considered exclusionary. It is recommended that participants are up to date with vaccinations before the first dose of IMP.
Lack of clinical response to plasmapheresis/plasma exchange (PLEX).
Positive serum test at screening for active viral infection with any of the following conditions: 1) Hepatitis B virus (HBV) indicative of an acute or chronic infection, unless associated with a negative HBV DNA test; 2) Hepatitis C virus (HCV) based on HCV antibody assay, unless associated with a negative HCV RNA test; 3) HIV based on test results associated with either: an AIDS-defining condition or a CD4 count of ≤200 cells/mm3 or no adequate treatment with antiretroviral therapy.
Any of the following prior therapy or procedures: 1) Treatment within 2 weeks before screening: topical corticosteroids or topical immunomodulators (e.g., tacrolimus) for IIM-related rash; 2) Treatment within 4 weeks before screening: local corticosteroid injections (intra articular, bursa, and tendon sheath injections), anakinra, etanercept, Janus kinase (JAK) inhibitors, intramuscular or IV corticosteroids, PLEX, immunoadsorption; 3) Treatment within 8 weeks before screening: SC or intramuscular corticosteroid precursors; 4) Treatment within 12 weeks before screening: IVIg, SCIg, tocilizumab, abatacept, infliximab, adalimumab, golimumab, certolizumab, ustekinumab; 5) Treatment within 24 weeks before screening: rituximab or other anti-CD20 antibody, cyclophosphamide; 6) Use of a nonbiologic investigational product within 12 weeks or 5 half-lives (whichever is longer) before screening, unless the drug washout is specified in the protocol; 7) Use of a biologic therapy and/or monoclonal antibody (including biologic investigational product) within 24 weeks or 5 half-lives (whichever is longer) before screening, unless the drug washout is specified in the protocol; 8) Treatment with OCS dose ≥20 mg prednisone or dose equivalent; 9) Treatment with ≥1 immunosuppressant, ≥1 antimalarial, or a combination of an immunosuppressant and an antimalarial.
Participant has previously participated in an efgartigimod clinical study and received at least 1 dose of IMP.
Participant is concurrently participating in any other clinical study, including a noninterventional study.
Participant has IgG <4 g/L at screening.
Participant has a current or history (i.e., within 12 months of screening) of alcohol, drug, or medication abuse.
Participant is pregnant or lactating or intends to become pregnant during the study.
Participant has severe renal impairment (estimated glomerular filtration rate ≤30 mL/min/1.73 m²) at screening.
Participant is institutionalized by a court or other governmental order or is in a dependent relationship with the sponsor or investigator.

### C. IMP(s) and Concomitant Therapy

Investigational medicinal product (IMP) is defined as any investigational intervention(s), marketed product(s), placebo, or medical device(s) intended to be administered to a study participant according to the study protocol.

### IMP(s) Administered

TABLE S1
IMP(s) administered

| | | |
|---|---|---|
| Intervention | Efgartigimod PH20 SC | Placebo |
| Label | | |
| Intervention | Efgartigimod PH20 SC | Placebo PH20 SC |
| Name | | |
| Intervention | Efgartigimod PH20 SC will be | Placebo PH20 SC will be provided as a |
| Description | provided as a sterile, clear to | sterile, colorless, clear solution for |
| | opalescent, yellowish solution for SC | injection in glass vials covered with a |
| | injection in glass vials covered with a | blinding shell, with the same |
| | blinding shell. | formulation as the efgartigimod PH20 |
| | | SC solution for injection, but without |
| | | the active ingredient (efgartigimod). |
| Type | Biologic | Other: placebo |
| Dose | Efgartigimod + 2000 U/mL rHuPH20 | Matching placebo + 2000 U/mL |
| Formulation | solution for SC injection to be dosed at | rHuPH20 solution for injection |
| | a fixed dose of 1000 mg per injection | |
| Unit Dose | 180 mg/mL efgartigimod (with | Placebo (with 2000 U/mL rHuPH20) |
| Strength(s) | 2000 U/mL rHuPH20) | |
| Dosage Level(s) | 1000 mg efgartigimod (and | Placebo (and approximately 11 000 U |
| | approximately 11 000 U rHuPH20) | rHuPH20) weekly |
| | weekly | |
| Route of | SC injection | SC injection |
| Administration | | |
| Use | Experimental | Placebo-comparator |
| IMP and AxMP | IMP | IMP |
| Sourcing | Provided centrally by the sponsor | Provided centrally by the sponsor |
| Packaging and | IMP will be provided in glass vials in | IMP will be provided in glass vials in |
| Labeling | secondary packaging boxes. Each vial | secondary packaging boxes. Each vial |
| | will be labeled and provided in | will be labeled and provided in |
| | secondary packaging as required per | secondary packaging as required per |
| | country requirements. Because there is | country requirements. Because there is a |
| | a slight color difference between | slight color difference between |
| | efgartigimod PH20 SC and placebo, | efgartigimod PH20 SC and placebo, |
| | each vial will be masked with a | each vial will be masked with a blinding |
| | blinding shell. | shell. |

AxMP = auxiliary medicinal product;
rHuPH20 = recombinant human hyaluronidase PH20;
SC = subcutaneous

Any medication or vaccine (including over the counter or prescription medicines, recreational drugs, vitamins, and/or herbal supplements [including Chinese traditional medicine]) or other specific categories of interest that the participant is receiving at the time of screening or receives during the study must be recorded and include the following information: 1) Reason for use; 2) Dates of administration including start and end dates; 3) Dosage information including dose and frequency.

The participant must be receiving background treatment for IIM and follow the requirements described. Allowed background treatments for IIM include any of the following: 1) Oral corticosteroid (OCS); 2) 1 antimalarial (hydroxychloroquine, quinacrine, or chloroquine); 3) 1 of the following immunosuppressants-methotrexate, azathioprine, mycophenolate mofetil, mycophenolic acid, tacrolimus, cyclosporine, leflunomide, or mizoribine (Japan only).

Participants must be taking at least 1 of these specified background treatments and may receive a combination of either OCS and up to 1 antimalarial or OCS and up to 1 immunosuppressant. A combination of an antimalarial and an immunosuppressant is not permitted. Administration of >1 medication with immunosuppressive properties, including Chinese traditional medications, is prohibited. If a participant is receiving Chinese traditional medicine to discuss any possible immunosuppressive properties.

An ongoing physical therapy or exercise program should follow the guidelines described.

Concurrent use of topical corticosteroid therapy for IIM-related rash is not permitted. Topical treatment for a non-IIM condition in the participant's medical history is permitted at a stable dose within 2 weeks from screening and throughout the study. Topical treatment is also permitted to treat a non-IIM related TEAE.

Intramuscular or IV corticosteroids are permitted for the treatment of a non-IIM-related TEAE if the requirements for corticosteroids are met. However, participants who require intramuscular or IV corticosteroids during screening will be considered screening failures.

Local corticosteroid injections are permitted to treat non-IIM-related TEAEs (e.g., local tendon, joint, bursal inflammation). However, participants who require a local corticosteroid injection during screening will be considered screening failures.

Statin use is permitted. Keeping the type and dose of statin consistent from screening to the end of the treatment period is advised. However, dose changes are permitted based on the investigator's judgment.

Analgesics and nonsteroidal anti-inflammatory drugs are permitted as required, based on the investigator's judgment.

Vaccines are permitted during the study except for live or live-attenuated vaccines. Vaccines must be documented as concomitant medications. Other types of vaccines are allowed at the discretion of the investigator during the study.

A corticosteroid rescue is defined as an OCS dose increase higher than the participant's baseline dose (e.g., a steroid burst). The guidance for rescue treatment is provided below.

### Background IIM Treatment

Immunosuppressants or antimalarials can be administered for a minimum of 12 weeks before screening, and at a stable dose for a minimum of 8 weeks before screening and then through baseline.

Participants who stopped treatment with an immunosuppressant (except for leflunomide) or an antimalarial are eligible if their last dose was >8 weeks before screening.

Participants who stopped treatment with leflunomide are eligible if their last dose was >12 weeks before screening unless leflunomide washout treatment was used, in which case participants are eligible if the washout treatment was completed >4 weeks before screening.

Dose regimens must remain stable from baseline to end of the treatment period but may be decreased for toxicity or to optimize management of an AE, such as infection. The toxicity/event must be confirmed as a documented AE. The dose can be returned to the baseline level if the toxicity/event resolves and if clinically indicated.

OCS (≤20 mg prednisone or dose equivalent) can be initiated >8 weeks before screening and must be at a stable dose for a minimum of 4 weeks before screening through baseline. Participants who stopped treatment with OCS before enrollment are eligible if the last dose was >4 weeks before screening.

All corticosteroid doses are expressed in equivalent prednisone dose.

Increasing the dose to more than the baseline dose is defined as a steroid burst. The increased dose must not exceed 20 mg more than the participant's baseline dose. The following sections describe the conditions under which steroid bursts are permitted.

The following schedule is recommended if the steroid burst is performed because of IIM disease worsening: the maximum dose (≤20 mg more than the baseline dose) for 3 days, then decrease the dose by up to 5 mg every 2 to 3 days.

Disease worsening criteria: Disease worsening is defined as when any of the following conditions occur (compared with baseline): MDGA worsening by ≥2 cm on the VAS and MMT8 worsening by ≥20%; global extramuscular activity worsening by ≥2 cm on the 10-cm MDAAT VAS; any 3 of 6 IMACS CSMs worsening by ≥30%.

IIM worsening must by documented in the worsening report. A non-IIM-related TEAE must be documented as an AE.

### Phase 2 Stage:

Through week 12: The dose must be kept stable, if possible, based on the participant's condition. A steroid burst is allowed if a participant meets the IIM disease worsening treatment described above (i.e., rescue treatment) or if needed for treatment of a non-IIM-related TEAE. The dose must be reduced to the participant's baseline dose within 14 days of the increase or no later than visit 4, whichever is sooner. A steroid burst starting less than 14 days before visit 4 must not extend past visit 4. One steroid burst is allowed only if there is an immediate risk to the participant's safety as determined by the investigator.

After week 12 through week 24: The dose must remain stable from visit 4 through visit 7. A dose increase is allowed only if there is an immediate risk to the participant's safety as determined by the investigator.

### Phase 3 Stage:

Through week 40: A steroid burst is allowed for IIM worsening (see above) and non-IIM-related TEAEs. A maximum of 2 steroid bursts are allowed through visit 11, but only 1 can be because of IIM worsening. The dose must be reduced to the participant's baseline dose within 14 days of the increase or no later than visit 11, whichever is sooner. A steroid burst starting less than 14 days before visit 11 must not extend past visit 11.

After week 40 to week 52: The participant's dose must remain stable from visit 11 through visit 14. Dose increase is allowed only if there is an immediate risk to the participant's safety as determined by the investigator.

### OCS Tapering:

### Phase 2 Stage:

Tapering of a participant's OCS dose is not permitted unless it is required to reduce toxicity or optimize management of an AE. The steroid toxicity or AE must be documented as an AE. The dose can be returned to baseline level if the toxicity or AE is resolved and if clinically indicated.

### Phase 3 Stage:

Through week 16: Tapering of a participant's OCS dose is not permitted unless it is required to reduce toxicity or optimize management of an AE. The steroid toxicity or AE must be documented as an AE. The dose can be returned to baseline level if the toxicity or AE is resolved and if clinically indicated.

After week 16 through week 40: OCS tapering must start at visit 5 and continue through visit 14 in participants receiving ≥5 mg of corticosteroids unless any of the disease worsening criteria are met. In this case, tapering can be delayed until the participant no longer meets these criteria. Tapering will be based on the participant's baseline dose and on prednisone equivalent doses if the participant is receiving other OCS. After the first decrease, participants will decrease their OCS by 2.5 mg every 4 weeks. The first OCS tapering dose may be less than a 2.5 mg dose reduction. For a baseline dose of 18 to 20 mg, the first decreased dose will be 17.5 mg. For a baseline dose of 16 to 17.5 mg, the first decreased dose will be 15 mg. For a baseline dose of 13 to 15 mg, then the first decreased dose will be 12.5 mg. For a baseline dose of 11 to 12.5 mg, the first decreased dose will be 10 mg. For a baseline dose of 8 to 9 mg, the first decreased dose will be 7.5 mg. For a starting dose of 6 to 7 mg, the first decreased dose will be 5 mg. Tapering is not required for participants receiving ≤5 mg of OCS. Tapering may continue to reduce safety or tolerability issues or if a decrease is otherwise clinically indicated.

A participant who has an increase in IIM disease activity when tapering is attempted may reverse the taper by ≤1 step based on the investigator's judgment. Reversing the taper by ≥1 step is recommended to reverse the taper by the minimum number of steps required to reduce IIM disease activity. Reversing the taper up to the participant's baseline OCS dose is not considered rescue treatment.

After week 40 through week 52: The participant's OCS dose must remain stable from visit 11 through visit 14. Decreases in the OCS dose after visit 11 are only permitted to reduce toxicity or optimize the management of an AE. The steroid toxicity or AE must be documented as an AE. The dose can be reverted to the week 40 dose if the toxicity or AE resolves and if it is clinically indicated.

### Prohibited Medications

The use of cyclophosphamide, any immunomodulating biologic agent, JAK inhibitors, immunoglobulin therapy, PLEX, any other experimental/study IMP, or live or live-attenuated vaccines are not permitted from their respective washout periods before enrollment through visit 7 for the phase 2 stage, visit 14 for the phase 3 stage, or the IMP discontinuation visit for participants who permanently discontinue IMP. Participants who use any of these prohibited therapies will be permanently discontinued from IMP. These restrictions do not apply to biologics that do not have an immunomodulatory effect (e.g., insulins). If the participant's condition mandates treatment with any prohibited medications based on the judgment of the investigator, IMP must be permanently discontinued before prohibited medication is administered.

### D. Study Assessments and Procedures

TABLE S2
Schedule of activities (Phase 2 stage)

| Study visit | | | | | | | | | | | | |
|---|---|---|---|---|---|---|---|---|---|---|---|---|
| | | | | | | | | | | IMP d/c | | Safety |
| SCR | V1/BL | Opt-in | V2 | V3 | V4 | V5 | V6 | V7^{a} | UNS^{b} | visit^{c} | EDV^{d} | follow-up^{e} |
| Study week | | | | | | | | | | | | |
| NA | 0 | NA | 4 | 8 | 12 | 16 | 20 | 24 | NA | NA | NA | NA |
| Study day (±day) | | | | | | | | | | | | |
| | | | | | | | | | | Next | Within 7 d | 56 d (±3 d) |
| | | 3 | 29 | 57 | 85 | 113 | 141 | 169 | | scheduled | (+3 d) after | after final |
| −28 to −1^{f} | 1 | (±1) | (±2) | (±2) | (±2) | (±2) | (±2) | (±2) | NA | visit | final dose | dose |

| | | | | | | | | | | | | |
|---|---|---|---|---|---|---|---|---|---|---|---|---|
| Informed consent | X | | | | | | | | | | | |
| Eligibility criteria | X | X^{g} | | | | | | | | | | |
| Demographic | X | | | | | | | | | | | |
| characteristics^{h} | | | | | | | | | | | | |
| Medical and | X | | | | | | | | | | | |
| surgical history | | | | | | | | | | | | |
| Adjudication committee | (X)ⁱ | | | | | | | | | | | |
| review of historical | | | | | | | | | | | | |
| muscle biopsy slides | | | | | | | | | | | | |
| 12-lead ECG^{j} | | X | | | X | | | X | (X) | X | X | X |
| Height | | X | | | | | | | | X | X | |
| Weight | X | X | | | | | | | (X) | X | X | |
| Physical | X | X | | | | | | | (X) | | | |
| examination (full) | | | | | | | | | | | | |
| Physical examination | | | X | X | X | X | X | X | (X) | X | X | X |
| (abbreviated)^{k} | | | | | | | | | | | | |
| Vital signs^{k} | X | X | X | X | X | X | X | X | (X) | X | X | X |
| MDI | X | X | | | | | | | (X) | | | |
| Randomization | | X | | | | | | | | | | |
| Dispense | | X | | | | | | | | | | |
| administration log | | | | | | | | | | | | |
| and patient diary | | | | | | | | | | | | |

TIS CSMs

| | | | | | | | | | | | |
|---|---|---|---|---|---|---|---|---|---|---|---|
| MMT8^{k, l} | X^{m} | X | X | X | X | X | X | X | (X) | X | X |
| HAQ-DI^{k} | X | X | X | X | X | X | X | X | (X) | X | X |
| MDGA^{k, l} | X | X | X | X | X | X | X | X | (X) | X | X |
| PGA^{k} | X | X | X | X | X | X | X | X | (X) | X | X |
| MDAAT^{k, l, n} | X | X | X | X | X | X | X | X | (X) | X | X |
| Blood sample for muscle | X | X | X | X | X | X | X | X | (X) | X | X |
| enzymes^{k, o} | | | | | | | | | | | |

Clinical efficacy parameters

| | | | | | | | | | | | |
|---|---|---|---|---|---|---|---|---|---|---|---|
| m30 STS | | X | | | X | | | X | (X) | X | X |
| Abbreviated handheld | | X | X | X | X | X | X | X | (X) | X | X |
| dynamometry | | | | | | | | | | | |
| CGI-S^{k, l} | | X | X | X | X | X | X | X | (X) | X | X |
| CGI-C^{l} | | | X | X | X | X | X | X | (X) | X | X |
| FI-3^{l} | | X | | | X | | | X | (X) | X | X |
| CDASI^{l, p} | X | X | | | X | | | X | (X) | X | X |

| | |
|---|---|
| Actigraphy measures^{q} | Recorded 7 days after BL and 7 days before V7 |

Patient-reported outcomes and quality of life assessments

| | | | | | | | | | | |
|---|---|---|---|---|---|---|---|---|---|---|
| PGI-S^{k} | X | X | X | X | X | X | X | (X) | X | X |
| PGI-C | | X | X | X | X | X | X | (X) | X | X |
| SF-36v2 | X | X | X | X | X | X | X | (X) | X | X |
| EQ-5D-5L | X | X | X | X | X | X | X | (X) | X | X |
| PROMIS Pain Interference | X | X | X | X | X | X | X | (X) | X | X |
| PROMIS Fatigue | X | X | X | X | X | X | X | (X) | X | X |
| PROMIS Physical Function | X | X | X | X | X | X | X | (X) | X | X |
| Pain NRS^{k} | X | X | X | X | X | X | X | (X) | X | X |
| Fatigue NRS^{k} | X | X | X | X | X | X | X | (X) | X | X |
| Pruritus NRS^{p} | X | X | X | X | X | X | X | (X) | X | X |
| Skindex-16^{p} | X | X | X | X | X | X | X | (X) | X | X |

Clinical laboratory assessments

| | | | | | | | | | | | | |
|---|---|---|---|---|---|---|---|---|---|---|---|---|
| Blood sample for clinical | X | X | X | X | X | X | X | X | (X) | X | X | X |
| laboratory tests^{k, o} | | | | | | | | | | | | |
| HBV, HCV, and HIV tests | X | | | | | | | | (X) | | | |
| COVID-19 test^{r} | (X) | | | | | | | (X) | (X) | | | |
| Serum pregnancy test^{s} | X | | | | | | | | (X) | | | |
| Urine pregnancy test^{j, s} | | X | X | X | X | X | X | X | (X) | X | X | X |
| Urinalysis^{j} | X | X | | | X | | | X | (X) | X | X | X |

Other blood samples^{o}

| | | | | | | | | | | | | | |
|---|---|---|---|---|---|---|---|---|---|---|---|---|---|
| MSA determination | X | | | | | | | | | (X) | | | |
| MSA/MAA titers^{j} | X | X | | X | X | X | X | X | X | (X) | X | X | X |
| PD (total IgG)^{j} | X^{t} | X^{t} | | X | X | X | X | X | X | (X) | X | X | X |
| Immunophenotyping | | X | | | | X | | | X | (X) | X | X | |
| of PBMC | | | | | | | | | | | | | |
| IFN score measurement^{u} | | X | | | | X | | | X | (X) | X | X | |
| IFN concentrations | | X | | | | X | | | X | (X) | X | X | |
| Serological markers of | | X | | | | X | | | X | (X) | X | X | |
| complement activation | | | | | | | | | | | | | |
| Immunogenicity^{j} | X^{t} | X^{t} | | X | X | X | X | X | X | (X) | X | X | X |
| PK^{j} | | X^{t} | X^{v} | X | X | X | X | X | X | (X) | X | X | X |
| MSA exploratory research | | X | | | | | | | | | | | |
| (optional) | | | | | | | | | | | | | |
| Future biomarker research | | X | | | | | | | | | | | |
| (optional) | | | | | | | | | | | | | |
| IMP administration | | (X) | | (X) | (X) | (X) | (X) | (X) | | | | | |
| training^{w} | | | | | | | | | | | | | |
| Review administration log | | | | X | X | X | X | X | X | (X) | X | X | |
| and patient diary | | | | | | | | | | | | | |
| IMP | | X | | X | X | X | X | X | | | | | |
| administration^{x} | | | | | | | | | | | | | |

| | |
|---|---|
| Concomitant therapies^{k} | Continuous monitoring from signing the ICF to last study-related activity |
| AEs^{k, y} | Continuous monitoring from signing the ICF to last study-related activity |

AE = adverse event;
BL = baseline visit;
CDASI = Cutaneous Dermatomyositis Disease Area and Severity Index;
CGI-C = Clinical Global Impression of Change;
CGI-S = Clinical Global Impression of Severity;
CSM = core set measure;
d/c = discontinuation;
DM = dermatomyositis;
ECG = electrocardiogram;
FI-3 = Functional Index-3;
HAQ-DI = Health Assessment Questionnaire Disability Index;
HBV = hepatitis B virus;
HCV = hepatitis C virus;
ICF = informed consent form;
IFN = interferon;
IgG = immunoglobulin G;
IIM = idiopathic inflammatory myopathy;
IMP = investigational medicinal product;
m30 STS = Modified 30-Second Sit-to-Stand Test;
MDAAT = Myositis Disease Activity Assessment Tool;
MDGA = physician global assessment of disease activity;
MDI = Myositis Damage Index;
MMT8 = manual muscle testing-8;
MSA = myositis-specific antibody;
NA = not applicable;
NRS = numeric rating scale;
OLE = open-label extension;
PBMC = peripheral blood mononuclear cell;
PCR = polymerase chain reaction;
PD = pharmacodynamics;
PGA = Patient Global Assessment of Disease Activity;
PGI-C = Patient Global Impression of Change;
PGI-S = Patient Global Impression of Severity;
PK = pharmacokinetics;
PROMIS = Patient-Reported Outcomes Measurement Information System;
SCR = screening;
SF-36v2 = 36-Item Short Form Survey version 2;
TEAE = treatment-emergent adverse event;
TIS = total improvement score;
UNS = unscheduled visit
Note:
The treatment period ends at visit 7. At this visit, participants will not receive IMP but may enroll in the OLE study ARGX-113-2011 if they are eligible. If the participant does not enroll in the OLE study, they will complete the safety follow-up visit.
Note:
(X) = visit activities that are optional or only required under specific circumstances. Refer to associated footnotes for more details.
^{a}This visit will be this study's final visit for participants rolling over to ARGX-113-2011.
^{b}It is advised that the 6 CSMs of the TIS be assessed during an unscheduled visit before changing the participant's corticosteroid dose to treat disease worsening. The investigator can decide which of the remaining assessments will be performed at each unscheduled visit.
^{c}Participants who permanently discontinue IMP will attend the IMP discontinuation visit and then attend every other visit of their regularly scheduled visits, without receiving IMP. The IMP discontinuation visit will be performed at the next scheduled visit after permanent IMP discontinuation. If a participant discontinues at a scheduled visit, that visit will become the IMP discontinuation visit.
^{d}The EDV visit is performed when participants discontinue the study.
^{e}The safety follow-up visit applies to all participants unless a participant has been temporarily interrupted from IMP and >56 days have elapsed since their final dose or enrolls in ARGX-113-2011. If IMP is permanently discontinued >56 days from week 24 and the participant agrees to continue with on-site prescheduled visits (refer to footnote k), the safety follow-up visit assessments will be performed at their scheduled visit closest to 56 days after their final dose.
^{f}Ad hoc extensions of the screening period may be permitted in certain circumstances (e.g., when screening laboratory results are pending or if the participant is unable to visit the site for the scheduled baseline [week 0] visit). Screening period extensions will only occur on a case-by-case basis with the sponsor's approval.
^{g}Laboratory eligibility criteria will be reviewed at screening. These data collected at baseline will not be used for determining eligibility.
^{h}Demographic characteristics comprise age, birth year, sex, race, and ethnicity (per local regulations). Race and ethnicity data will be source verified only if permitted by local laws.
ⁱThese activities will only be performed if specific eligibility criteria apply.
^{j}For participants who permanently discontinue IMP, the 12-lead ECG, the urine pregnancy test, urinalysis, the MSA/MAA titers, PD, immunogenicity, and PK assessments only need to be performed at the IMP discontinuation visit and the scheduled visit closest to 56 days (±3 days) after the final dose.
^{k}Participants permanently discontinuing IMP attending every other visit of their scheduled on-site visits will undergo the following assessments: abbreviated physical examination, vital signs, MMT8, HAQ-DI, MDGA, PGA, the extramuscular global assessment of the MDAAT, muscle enzymes, CGI-S, PGI-S, Pain NRS, Fatigue NRS, clinical laboratory tests, concomitant therapy monitoring, and AE monitoring. Participants performing phone visits will only undergo monitoring for concomitant medication and AEs. All participants who permanently discontinue IMP and remain in the study will perform an on-site visit at week 24.
^{l}A participant's disease activity assessments must be performed by the same individual throughout the study, unless it is not feasible to do so. All disease activity assessments except the m30 STS and the FI-3 must be performed by a physician; the m30 STS and FI3 must be performed by a trained rater.
^{m}At screening, the following muscles should be assessed for weakness (in addition to those tested in the MMT8): wrist flexors, finger flexors (flexor digitorum profundus, flexor pollicis longus), and finger extensors. All 15 MMT8 muscle groups must be assessed at screening to determine eligibility. If any of the MMT8 muscle groups are not assessable, the participant will not be considered eligible.
ⁿFollowing IMP discontinuation, only the extramuscular global assessment of the MDAAT needs to be performed.
^{o}Blood samples must be taken predose on dosing days, preferably within 2 hours before administering IMP. It is recommended that blood samples are collected before the MMT8 and other muscle assessments.
^{p}Assessment will be performed on only participants with DM. Both the CDASI activity and damage scores will be calculated. However, only the activity score will be considered for the participant's eligibility, per inclusion criterion 4.
^{q}Actigraphy measures include step counts, cadence, vector magnitude, and time spent in different levels of daily activities (mild to moderate to strenuous activity and sedentary activity).
^{r}Participants will be tested for SARS-CoV-2 if they are symptomatic or if applicable law requires testing; if applicable, a negative PCR test (central or local laboratory) is required within 72 hours before enrollment and should occur regardless of a participant's vaccination status. Week 24 (V7) COVID-19 testing applies only to participants who will enroll in ARGX-113-2011 and only if the participant is symptomatic or applicable law requires testing.
^{s}Serum and urine pregnancy tests apply only to women of childbearing potential.
^{t}Blood samples may also be used to cross-validate the PK, PD, and immunogenicity assays in the IIM matrix (serum and plasma).
^{u}Refer to genetics assessment described herein.
^{v}This optional PK blood sampling should preferably occur at the site, but if needed, the sample may be taken at the participant's home by a qualified person.
^{w}Training sessions for the participant or their caregiver can occur at any time during the study.
^{x}IMP will be administered weekly. At scheduled visits, the administration will be performed by site staff after all visit activities have been completed. The last administration of the double-blinded treatment period is at week 23 (study day 162 ± 2 days).
^{y}Any AEs that are ongoing at the time when a participant enrolls in ARGX-113-2011 will be followed up within the OLE study.

TABLE S3
Schedule of activities (Phase 3 stage)

| | Study visit | | | | | | | | | | |
|---|---|---|---|---|---|---|---|---|---|---|---|
| | SCR | V1/BL | V2 | V3 | V4 | V5 | V6 | V7 | V8 | V9 | V10 |
| | Study week | | | | | | | | | | |
| | NA | 0 | 4 | 8 | 12 | 16 | 20 | 24 | 28 | 32 | 36 |
| | Study day (±day) | | | | | | | | | | |
| | | | 29 | 57 | 85 | 113 | 141 | 169 | 197 | 225 | 253 |
| | −28 to −1^{f} | 1 | (±2) | (±2) | (±2) | (±2) | (±2) | (±2) | (±2) | (±2) | (±2) |
| Informed consent | X | | | | | | | | | | |
| Eligibility criteria | | X^{g} | | | | | | | | | |
| Demographic | X | | | | | | | | | | |
| characteristics^{h} | | | | | | | | | | | |
| Medical and surgical | X | | | | | | | | | | |
| history | | | | | | | | | | | |
| Adjudication committee | (X)ⁱ | | | | | | | | | | |
| review of historical | | | | | | | | | | | |
| muscle biopsy slides | | | | | | | | | | | |
| 12-lead ECG^{j} | | X | | | X | | | X | | | X |
| Height | | X | | | | | | | | | |
| Weight | X | X | | | | | | X | | | |
| Physical examination | X | X | | | | | | | | | |
| (full) | | | | | | | | | | | |
| Physical examination | | | X | X | X | X | X | X | X | X | X |
| (abbreviated)^{k} | | | | | | | | | | | |
| Vital signs^{k} | X | X | X | X | X | X | X | X | X | X | X |
| MDI | X | X | | | | | | | | | |
| Randomization | | X | | | | | | | | | |
| Dispense administration | | X | | | | | | | | | |
| log and patient diary | | | | | | | | | | | |

TIS CSMs

| | | | | | | | | | | | |
|---|---|---|---|---|---|---|---|---|---|---|---|
| MMT8^{k, l} | X^{m} | X | X | X | X | X | X | X | X | X | X |
| HAQ-DI^{k} | X | X | X | X | X | X | X | X | X | X | X |
| MDGA^{k, l} | X | X | X | X | X | X | X | X | X | X | X |
| PGA^{k} | X | X | X | X | X | X | X | X | X | X | X |
| MDAAT^{k, l, n} | X | X | X | X | X | X | X | X | X | X | X |
| Blood sample for | X | X | X | X | X | X | X | X | X | X | X |
| muscle enzymes^{k, o} | | | | | | | | | | | |

Clinical efficacy parameters

| m30 STS | | X | X | | | X | | | X | | |
|---|---|---|---|---|---|---|---|---|---|---|---|
| Abbreviated handheld | | X | X | X | X | X | X | X | X | X | X |
| dynamometry | | | | | | | | | | | |
| CGI-S^{k, l} | | X | X | X | X | X | X | X | X | X | X |
| CGI-C^{l} | | | X | X | X | X | X | X | X | X | X |
| FI-3^{l} | | X | X | | | X | | | X | | |
| CDASI^{l, p} | X | X | X | | | X | | | X | | |
| Actigraphy measures^{q} | | Recorded 7 days after BL, 7 days before week 24, and 7 days before week 52 | | | | | | | | | |
| C-GTI | | X | | | | | | X | | | |

Patient-reported outcomes and quality of life assessments

| | | | | | | | | | | |
|---|---|---|---|---|---|---|---|---|---|---|
| PGI-S^{k} | X | X | X | X | X | X | X | X | X | X |
| PGI-C | | X | X | X | X | X | X | X | X | X |
| SF-36v2 | X | X | X | X | X | X | X | X | X | X |
| EQ-5D-5L | X | X | X | X | X | X | X | X | X | X |
| PROMIS Pain | X | X | X | X | X | X | X | X | X | X |
| Interference | | | | | | | | | | |
| PROMIS Fatigue | X | X | X | X | X | X | X | X | X | X |
| PROMIS Physical | X | X | X | X | X | X | X | X | X | X |
| Function | | | | | | | | | | |
| Pain NRS^{k} | X | X | X | X | X | X | X | X | X | X |
| Fatigue NRS^{k} | X | X | X | X | X | X | X | X | X | X |
| Pruritus NRS^{p} | X | X | X | X | X | X | X | X | X | X |
| Skindex-16^{p} | X | X | X | X | X | X | X | X | X | X |

Clinical laboratory assessments

| | | | | | | | | | | | |
|---|---|---|---|---|---|---|---|---|---|---|---|
| Blood sample for | X | X | X | X | X | X | X | X | X | X | X |
| clinical laboratory | | | | | | | | | | | |
| tests^{k, o} | | | | | | | | | | | |
| HBV, HCV, and HIV | X | | | | | | | | | | |
| tests | | | | | | | | | | | |
| COVID-19 test^{r} | (X) | | | | | | | | | | |
| Serum pregnancy test^{s} | X | | | | | | | | | | |
| Urine pregnancy test^{j, s} | | X | X | X | X | X | X | X | X | X | X |
| Urinalysis^{j} | X | X | X | | | X | | | X | | |

Other blood samples^{o}

| | | | | | | | | | | | |
|---|---|---|---|---|---|---|---|---|---|---|---|
| MSA determination | X | | | | | | | | | | |
| MSA/MAA titers^{j} | X | X | X | X | X | X | X | X | X | X | X |
| PD (total IgG)^{j} | X^{t} | X^{t} | X | X | X | X | X | X | X | | X |
| Immunophenotyping | | X | | | X | | | X | | | |
| of PBMC | | | | | | | | | | | |
| IFN score | | X | | | X | | | X | | | |
| measurement^{u} | | | | | | | | | | | |
| IFN concentrations | | X | | | X | | | X | | | |
| Serological markers | | X | | | X | | | X | | | |
| of complement | | | | | | | | | | | |
| activation | | | | | | | | | | | |
| Immunogenicity^{j} | X^{t} | X^{t} | X | X | X | X | X | X | X | | X |
| PK^{j} | | X^{t} | X | X | X | X | X | X | X | | X |
| MSA exploratory | | X | | | | | | | | | |
| research (optional) | | | | | | | | | | | |
| Future biomarkers | | X | | | | | | | | | |
| research (optional) | | | | | | | | | | | |
| IMP administration | | (X) | (X) | (X) | (X) | (X) | (X) | (X) | (X) | (X) | (X) |
| training^{v} | | | | | | | | | | | |
| Review administration | | | X | X | X | X | X | X | X | X | X |
| log and patient diary | | | | | | | | | | | |
| IMP administration^{w} | | X | X | X | X | X | X | X | X | X | X |

| | |
|---|---|
| Concomitant therapies^{k} | Continuous monitoring |
| Adverse events^{k, x} | Continuous monitoring |

| | Study visit | | | | | | | |
|---|---|---|---|---|---|---|---|---|
| | | | | | | IMP d/c | | Safety |
| | V11 | V12 | V13 | V14^{a} | UNS^{b} | visit^{c} | EDV^{d} | follow-up^{e} |
| | Study week | | | | | | | |
| | 40 | 44 | 48 | 52 | NA | NA | NA | NA |
| | Study day (±day) | | | | | | | |
| | | | | | | Within | Within | |
| | | | | | | 7 d | 7 d | 56 d |
| | | | | | | (+3 d) | (+3 d) | (±3 d) |
| | | | | | | after | after | after |
| | 281 | 309 | 337 | 365 | | final | final | final |
| | (±2) | (±2) | (±2) | (±2) | NA | dose | dose | dose |
| Informed consent | | | | | | | | |
| Eligibility criteria | | | | | | | | |
| Demographic | | | | | | | | |
| characteristics^{h} | | | | | | | | |
| Medical and surgical | | | | | | | | |
| history | | | | | | | | |
| Adjudication committee | | | | | | | | |
| review of historical | | | | | | | | |
| muscle biopsy slides | | | | | | | | |
| 12-lead ECG^{j} | | | X | X | (X) | X | | X |
| Height | | | | X | | X | | |
| Weight | | | | X | (X) | X | | |
| Physical examination | | | | | (X) | | | |
| (full) | | | | | | | | |
| Physical examination | X | X | X | X | (X) | X | | X |
| (abbreviated)^{k} | | | | | | | | |
| Vital signs^{k} | X | X | X | X | (X) | X | | X |
| MDI | | | | | (X) | | | |
| Randomization | | | | | | | | |
| Dispense administration | | | | | | | | |
| log and patient diary | | | | | | | | |
| TIS CSMs | | | | | | | | |
| MMT8^{k, l} | X | X | X | X | (X) | X | | |
| HAQ-DI^{k} | X | X | X | X | (X) | X | | |
| MDGA^{k, l} | X | X | X | X | (X) | X | | |
| PGA^{k} | X | X | X | X | (X) | X | | |
| MDAAT^{k, l, n} | X | X | X | X | (X) | X | | |
| Blood sample for | X | X | X | X | (X) | X | | |
| muscle enzymes^{k, o} | | | | | | | | |
| Clinical efficacy parameters | | | | | | | | |
| m30 STS | X | | | X | (X) | X | | |
| Abbreviated handheld | X | X | X | X | (X) | X | | |
| dynamometry | | | | | | | | |
| CGI-S^{k, l} | X | X | X | X | (X) | X | | |
| CGI-C^{l} | X | X | X | X | (X) | X | | |
| FI-3^{l} | X | | | X | (X) | X | | |
| CDASI^{l, p} | X | | | X | (X) | X | | |
| Actigraphy measures^{q} | Recorded 7 days after BL, 7 days before | | | | (X) | | | |
| | week 24, and 7 days before week 52 | | | | | | | |
| C-GTI | | | | X | | X | | |
| Patient-reported outcomes and quality of life assessments | | | | | | | | |
| PGI-S^{k} | X | X | X | X | (X) | X | | |
| PGI-C | X | X | X | X | (X) | X | | |
| SF-36v2 | X | X | X | X | (X) | X | | |
| EQ-5D-5L | X | X | X | X | (X) | X | | |
| PROMIS Pain | X | X | X | X | (X) | X | | |
| Interference | | | | | | | | |
| PROMIS Fatigue | X | X | X | X | (X) | X | | |
| PROMIS Physical | X | X | X | X | (X) | X | | |
| Function | | | | | | | | |
| Pain NRS^{k} | X | X | X | X | (X) | X | | |
| Fatigue NRS^{k} | X | X | X | X | (X) | X | | |
| Pruritus NRS^{p} | X | X | X | X | (X) | X | | |
| Skindex-16^{p} | X | X | X | X | (X) | X | | |
| Clinical laboratory assessments | | | | | | | | |
| Blood sample for | X | X | X | X | (X) | X | | X |
| clinical laboratory | | | | | | | | |
| tests^{k, o} | | | | | | | | |
| HBV, HCV, and HIV | | | | | (X) | | | |
| tests | | | | | | | | |
| COVID-19 test^{r} | | | | (X) | (X) | | | |
| Serum pregnancy test^{s} | | | | | (X) | | | |
| Urine pregnancy test^{j, s} | X | X | X | X | (X) | X | | X |
| Urinalysis^{j} | X | | | X | (X) | X | | X |
| Other blood samples^{o} | | | | | | | | |
| MSA determination | | | | | (X) | | | |
| MSA/MAA titers^{j} | X | X | X | X | (X) | X | | X |
| PD (total IgG)^{j} | | X | | X | (X) | X | | X |
| Immunophenotyping | | | | X | (X) | X | | |
| of PBMC | | | | | | | | |
| IFN score | | | | X | (X) | X | | |
| measurement^{u} | | | | | | | | |
| IFN concentrations | | | | X | (X) | X | | |
| Serological markers | | | | X | (X) | X | | |
| of complement | | | | | | | | |
| activation | | | | | | | | |
| Immunogenicity^{j} | | X | | X | (X) | X | | X |
| PK^{j} | | X | | X | (X) | X | | X |
| MSA exploratory | | | | | | | | |
| research (optional) | | | | | | | | |
| Future biomarkers | | | | | | | | |
| research (optional) | | | | | | | | |
| IMP administration | (X) | (X) | (X) | | | | | |
| training^{v} | | | | | | | | |
| Review administration | X | X | X | | (X) | | | |
| log and patient diary | | | | | | | | |
| IMP administration^{w} | X | X | X | | | | | |

| | |
|---|---|
| Concomitant therapies^{k} | Continuous monitoring |
| Adverse events^{k, x} | Continuous monitoring |

AE = adverse event;
C-GTI = composite glucocorticoid toxicity index;
CDASI = Cutaneous Dermatomyositis Disease Area and Severity Index;
CGI-C = Clinical Global Impression of Change;
CGI-S = Clinical Global Impression of Severity;
CSM = core set measure;
d/c = discontinuation;
DM = dermatomyositis;
ECG = electrocardiogram;
FI-3 = Functional Index-3;
HAQ-DI = Health Assessment Questionnaire Disability Index;
HBV = hepatitis B virus;
HCV = hepatitis C virus;
IFN = interferon;
IgG = immunoglobulin G;
IIM = idiopathic inflammatory myositis;
IMP = investigational medicinal product;
m30 STS = Modified 30-Second Sit-to-Stand Test;
MDAAT = Myositis Disease Activity Assessment Tool;
MDGA = physician global assessment of disease activity;
MDI = Myositis Damage Index;
MMT8 = manual muscle testing-8;
MSA = myositis-specific antibody;
NA = not applicable;
NRS = numeric rating scale;
OLE = open-label extension;
PD = pharmacodynamics;
PGA = Patient Global Assessment of Disease Activity;
PGI-C = Patient Global Impression of Change;
PGI = S = Patient Global Impression of Severity;
PK = pharmacokinetics;
PROMIS = Patient-Reported Outcomes Measurement Information System;
SCR = screening;
SF-36v2 = 36-Item Short Form Survey version 2;
TEAE = treatment-emergent adverse event;
TIS = total improvement score;
UNS = unscheduled visit
Note:
The treatment period ends at visit 14. At this visit, participants will not receive IMP, but may enroll in the OLE study ARGX-113-2011 if they are eligible. If the participant does not enroll in the OLE study, they will complete the safety follow-up visit.
Note:
(X) = visit activities that are optional or only required under specific circumstances. Refer to associated footnotes for more details.
^{a}This visit will be this study's final visit for participants rolling over to ARGX-113-2011.
^{b}It is advised that the 6 CSMs of the TIS be assessed during an unscheduled visit before changing the participant's corticosteroid dose to treat disease worsening. The investigator can decide which of the remaining assessments will be performed at each unscheduled visit.
^{c}Participants who permanently discontinue IMP will attend the IMP discontinuation visit and then attend every other visit of their regularly scheduled visits, without receiving IMP. The IMP discontinuation visit will be performed at the next scheduled visit after permanent IMP discontinuation. If a participant discontinues at a scheduled visit, that visit will become the IMP discontinuation visit.
^{d}The EDV visit is performed when participants discontinue the study.
^{e}The safety follow-up visit applies to all participants unless a participant has been temporarily interrupted from IMP and >56 days have elapsed since their final dose or enrolls in ARGX-113-2011. If IMP is permanently discontinued >56 days from week 52 and the participant agrees to continue with on-site prescheduled visits (refer to footnote k), the safety follow-up visit assessments will be performed at their scheduled visit closest to 56 days after their final dose.
^{f}Ad hoc extensions of the screening period may be permitted in certain circumstances (e.g., when screening laboratory results are pending or if the participant is unable to visit the site for the scheduled baseline [week 0] visit). Screening period extensions will only occur on a case-by-case basis with the sponsor's approval.
^{g}Laboratory eligibility criteria will be reviewed at screening. These data collected at baseline will not be used for determining eligibility.
^{h}Demographic characteristics comprise age, birth year, sex, race, and ethnicity (per local regulations). Race and ethnicity data will be source verified only if permitted by local laws.
ⁱThese activities will only be performed if specific eligibility criteria apply.
^{j}For participants who permanently discontinue IMP, the 12-lead ECG, the urine pregnancy test, urinalysis, the MSA/MAA titers, PD, immunogenicity, and PK assessments only need to be performed at the IMP discontinuation visit and the scheduled visit closest to 56 days (±3 days) after the final dose.
^{k}Participants permanently discontinuing IMP attending every other visit of their scheduled on-site visits will undergo the following assessments: abbreviated physical examination, vital signs, MMT8, HAQ-DI, MDGA, PGA, the extramuscular global assessment of the MDAAT, muscle enzymes, CGI-S, PGI-S, Pain NRS, Fatigue NRS, clinical laboratory tests, concomitant therapy monitoring, and AE monitoring. Participants performing phone visits will only undergo monitoring for concomitant medication and AEs. All participants who permanently discontinue IMP and remain in the study will perform an on-site visit at week 52.
^{l}A participant's disease activity assessments must be performed by the same individual throughout the study, unless it is not feasible to do so. All disease activity assessments except the m30 STS and the FI-3 must be performed by a physician; the m30 STS and FI3 must be performed by a trained rater.
^{m}At screening, the following muscles should be assessed for weakness (in addition to those tested in the MMT8): wrist flexors, finger flexors (flexor digitorum profundus, flexor pollicis longus), and finger extensors. All 15 MMT8 muscle groups must be assessed at screening to determine eligibility. If any of the MMT8 muscle groups are not assessable, the participant will not be considered eligible.
ⁿFollowing IMP discontinuation, only the extramuscular global assessment of the MDAAT needs to be performed.
^{o}Blood samples must be taken predose on dosing days, preferably within 2 hours before administering IMP. It is recommended that blood samples are collected before the MMT8 and other muscle assessments.
^{p}Assessment will be performed on only participants with DM. Both the CDASI activity and damage scores will be calculated. However, only the activity score will be considered for the participant's eligibility, per inclusion criterion 4.
^{q}Actigraphy measures include step counts, cadence, vector magnitude, and time spent in different levels of daily activities (mild to moderate to strenuous activity and sedentary activity).
^{r}Participants will be tested for SARS-CoV-2 if they are symptomatic or if applicable law requires testing; if applicable, a negative PCR test (central or local laboratory) is required within 72 hours before enrollment and should occur regardless of a participant's vaccination status. Week 52 (V14) COVID-19 testing applies only to participants who will enroll in ARGX-113-2011 and only if the participant is symptomatic or applicable law requires testing.
^{s}Serum and urine pregnancy tests apply only to women of childbearing potential.
^{t}Blood samples may also be used to cross-validate the PK, PD, and immunogenicity assays in the IIM matrix (serum and plasma).
^{u}Refer to genetics assessment described herein.
^{v}Training sessions for the participant or their caregiver can occur at any time during the study.
^{w}IMP will be administered weekly. At scheduled investigative site visits, the administration will be performed by site staff after all visit activities have been completed. The last administration of the double-blinded treatment period is at week 51 (study day 358 ± 2 days).
^{x}Any AEs that are ongoing at the time when a participant enrolls in ARGX-113-2011 will be followed up within the OLE study.

### TIS and CSMs

The TIS assesses minimal, moderate, and major clinical response, and is assessed using the ACR/EULAR criteria.

The ACR/EULAR criteria calculates TIS using the 6 CSMs: MDGA, PGA, MMT8, HAQ-DI, muscle enzymes, and extramuscular global assessment (assessed by the Myositis Disease Activity Assessment Tool [MDAAT]).

The absolute percentage change from baseline in each measure with varying weights is combined to obtain a TIS on a scale from 0 to 100. Higher scores indicate greater improvement. Improvements are defined by the following increases in TIS: Minimal improvement—at least 20 points; Moderate improvement—at least 40 points; Major improvement—at least 60 points.

### MDGA

The MDGA is a tool that measures the physician's global evaluation of the participant's overall disease activity, defined as potentially reversible pathology or physiology resulting from IIMN. The physician rates disease activity on the MDGA using a 10-cm VAS.

Overall disease activity is rated by drawing a vertical mark on a 10-cm VAS from the left end of the line (no evidence of disease activity), midpoint of the line (moderate disease activity), and the right end of the line (extremely active or severe disease activity).

Data will not be collected for the MDGA Likert scale in this study.

### PGA

The PGA is a tool that measures a patient's global evaluation of their overall disease activity at the time of assessment using a 10-cm VAS.

The participant rates their overall disease activity by drawing a vertical mark on a 10-cm VAS from the left end of the line (no evidence of disease activity) to the right end of the line (extremely active or severe disease activity).

### MMT8

The MMT8 is a physician assessment of muscle strength in a set of 8 designated muscles tested bilaterally (proximal muscles [deltoids, biceps, gluteus maximus, gluteus medius, and quadriceps] and distal muscles [wrist extensors and ankle dorsiflexors], potential score 0 to 140) and axially (neck flexors, potential score 0 to 10). The MMT8 is scored according to the Kendall scale. The highest total potential MMT8 score is 150. All 15 MMT8 muscles groups must be assessed at screening to determine eligibility. If any of the MMT8 muscle groups are not assessable at screening, the participant will not be considered eligible. The following muscles should be assessed for weakness at screening (in addition to those tested in the MMT8): wrist flexors, finger flexors (flexor digitorum profundus, flexor pollicis longus), and finger extensors.

### HAQ-DI

The HAQ-DI assesses physical function and is recommended by IMACS as a CSM in therapeutic studies reporting on myositis disease activity.

The participant assesses their usual abilities within the past week in 8 categories: dressing and grooming, arising, walking, reach, eating, hygiene, grip, and activities. Each category has 2 to 3 questions scored by the participant using a 4-point scale from 0 (without any difficulty) to 3 (unable to do). Scores may be adjusted based on the participant's responses to separate questions regarding their need for aids and devices or help from another person.

### Muscle Enzymes

Muscle-associated enzymes include CK, the transaminases (ALT, AST), LDH, and aldolase. These enzymes will be measured using validated methods from blood sampled as indicated in Tables 2 and 3. The actual date and time of the blood sample collection will be collected and included in the central laboratory data transfer.

### MDAAT

The MDAAT is a combined tool that includes the MYOACT, which is a series of physician's assessments of disease activity of various organ systems modified from the Vasculitis Activity Index, and the MITAX, which is modified from the British Isles Lupus Assessment Group approach to assess disease activity in lupus.

Among the MYOACT components of the MDAAT assessments, the extramuscular global assessment is a CSM of the TIS and is a tool used by the physician for an overall evaluation of disease activity in all the extramuscular organ systems. This assessment specifically excludes muscle disease activity. The physician uses a 10-cm VAS to rate the participant's overall disease activity in all extramuscular systems within the past 4 weeks that are caused by active IIM disease.

The MITAX component of the MDAAT assesses disease activity of various organ systems by assessing 26 clinical features within the past 4 weeks that are caused by active disease, using a 5 point scale: 0=not present in the past 4 weeks; 1=improving; 2=the same; 3=worse; 4=new.

### PGI-S and PGI-C

The PGI-S and PGI-C are simple, valid, participant-rated, single-item global measures of their condition.

The PGI-S asks participants to rate the severity of their disease symptoms over the past 7 days on a 4-point Likert scale, where scores range from “no symptoms” to “severe.”

The PGI-C asks participants to rate how much their overall status has changed from the start of the study on a 7-point Likert scale, where scores range from “much better” to “much worse.”

### CGI-S and CGI-C

The CGI-S and CGI-C are simple, valid, physician-rated, single-item global measures of the participant's disease.

The CGI-S asks the physician to rate the severity of the participant's disease on a 4-point Likert scale, where scores range from “no activity” to “severe.”

The CGI-C asks the physician to rate how much the participant's overall disease has changed from the start of the study on a 7-point Likert scale, where scores range from “much better” to “much worse.”

### FI-3

The FI-3 is a validated method for assessing functional disability (i.e., muscle endurance and stamina) in patients with DM or PM. Participants will attempt to perform 3 different muscle group tasks: head lift (neck flexion), shoulder flexion, and hip flexion.

The movement pace for each task will be standardized using a metronome (40 beats/min). Participants will attempt to perform up to 60 hip and shoulder flexion repetitions within 3 minutes and up to 30 head flexion repetitions within 1.5 minutes. After performing each task, participants will rate their perceived muscle exertion on the Borg CR-10 scale from 0 (no exertion) to 10 (maximal exertion).

### PROMIS Assessments

PROMIS is a publicly available system of highly reliable, precise measures of patient-reported health status for physical, mental, and social well-being. PROMIS instruments measure concepts including pain, fatigue, and physical function.

PROMIS Fatigue (Short Form 7a) instrument assesses the impact and experience of fatigue over the past 7 days. This validated 7-question scale has 5 response options, with scores ranging from 1 to 5. Scores are converted to a T-score, and higher scores indicate higher levels of fatigue. A decrease in score (negative change from baseline) indicates improvement in fatigue.

The PROMIS Pain Interference (Short Form 6a) instrument measures the self-reported consequences of pain across aspects of life, including social, cognitive, emotional, physical, and recreational activities over the past 7 days. This validated 6-question scale has 5 response options, with scores ranging from 1 to 5. Scores are converted to a T-scores, and higher scores indicate greater pain interference. A decrease in score (negative change from baseline) indicates improvement in pain interference.

The PROMIS Physical Function (Short Form 8b) instrument measures self-reported capability rather than actual performance of physical activities. A single physical function capability score is obtained from a short form. This validated 8-question scale has 5 response options, with scores ranging from 1 to 5. Scores are converted to T-scores, and higher scores indicate better self-reported capability in physical function. An increase in score (positive change from baseline) indicates improvement in self-reported physical function.

### Pain NRS

Participants will record a score (0 to 10) for worst pain experienced within the past 7 days

### Fatigue NRS

Participants will record a score (0 to 10) for worst physical fatigue experienced within the past 7 days.

### SF-36v2

The SF-36v2 is a 36-item scale constructed to survey health-related quality of life on 8 domains: 1) Limitations in physical activities caused by health problems; 2) Limitations in social activities caused by physical or emotional problems; 3) Limitations in usual role activities caused by physical health problems; 4) Bodily pain; 5) General mental health (psychological distress and well-being); 6) Limitations in usual role activities caused by emotional problems; 7) Vitality (energy and fatigue); 8) General health perceptions.

### EQ-5D-5L

The EQ-5D-5L questionnaire is a standardized test recognized by many health authorities as a generic measure of health status for clinical and economic appraisal.

The descriptive system comprises 5 dimensions: 1) Mobility; 2) Self-care; 3) Usual activities; 4) Pain/discomfort; 5) Anxiety/depression.

Each dimension has 5 levels: 1) No problem; 2) Slight problem; 3) Moderate problem; 4) Severe problem; 5) Extreme problem.

The participant will be asked to indicate his/her health state by ticking the (or placing a cross in) the box against the most appropriate statement in each of the 5 dimensions. This decision results in a 1-digit number expressing the level selected for that dimension. The digits for 5 dimensions are combined in a 5-digit number describing the respondent's health state. A unique health state is defined by combining 1 level from each of the 5 dimensions. A total of 3125 possible health states could be defined in this way. Each state is referred to in terms of a 5-digit code. For example, state 11111 would indicate no problems in any of the 5 dimensions, and state 12345 would indicate no problem with mobility, slight problems with washing or dressing, moderate problems with doing usual activities, severe pain or discomfort, and extreme anxiety or depression.

A VAS is included in the EQ-5D-5L. Participants will mark their health status from 0 (the worst health you can imagine) to 100 (the best health you can imagine) on the day the interview is conducted.

m30 STS test

The m30 STS test measures proximal muscle weakness. Participants will be seated in a standard chair with armrests and instructed to stand up and then sit down again for 30 seconds. Participants may use their hands and arms to help stand if needed. The number of sit-to-stand repetitions will be recorded.

### Monitor Actigraphy

Participants will be instructed to wear an actigraph (a physical activity monitor) at the times specified in the SoA. The actigraph will record step counts, cadence, vector magnitude, and time spent in different levels of daily activities (mild, moderate, to strenuous activity, and sedentary activity).

### Abbreviated Handheld Dynamometry

Handheld dynamometry measures the peak isometric force generated from a muscle group and is used to quantify muscle strength in patients with JIM. Muscle strength will be assessed by handheld dynamometry in the deltoid and iliopsoas bilaterally using a provided dynamometer and according to methods designated in the study manual.

CDASI (Participants with DM Only)

CDASI is a skin-specific outcome measure used to assess disease in patients with DM. Disease in 15 different anatomical locations is rated using 3 activity measures (erythema, scale, erosion/ulceration) and 2 damage measures (poikiloderma, calcinosis). The 3 activity measures on the 15 anatomical locations can add up to 90 points; Gottron's papules on the hands, periungual, and alopecia can add up to 10 points. The 2 damage measures on the 15 anatomical locations can add up to 30 points with 2 additional points for Gottron's hands. CDASI also assesses the presence and severity of Gottron's papules, periungual changes, and alopecia. The resulting activity and damage scores range from 0 to 100 and 0 to 32, respectively. Higher scores indicate greater disease severity.

Pruritis NRS (Participants with DM Only)

Pruritic symptoms of DM will be indicated by the participant on the Pruritis NRS, recording an average and a worst score (0 to 10) for itch experienced within the past 24 hours.

Skindex-16 (Participants with DM Only)

The Skindex-16 is a single-page survey that assesses how the symptoms, emotions, and functioning from the cutaneous symptoms of DM affect the participant's quality of life. Using a Likert scale of 0 (never) to 6 (always), the participant rates how often of each skin disease effect has been over the preceding week. Skindex-16 provides a single score for each domain (symptoms, emotions, and functioning) and an average score.

### MDI

The MDI is a tool used for physician assessment of the extent and severity of disease damage in 11 organ systems. Only the muscle damage VAS will be used in this study, where the severity of damage is measured by a physician's overall rating of disease damage to the muscular system, using a 10-cm VAS.

Damage is defined as a persistent/permanent change in anatomy, physiology, pathology or function, which is considered to have occurred after the diagnosis of myositis and has been present for at least 6 months. Damage may be the result of prior active disease (causing scarring, fibrosis and atrophy), complications of therapy, co-morbid conditions, or other events. A portion of disease damage is disease chronicity. Features of damage are ascertained by clinical assessment and must be present for at least 6 months (or the pathology that led to the feature must have been present for at least 6 months) despite prior immunosuppressive or other therapy, including exercise and rehabilitation. Examples of muscle damage include, but are not limited to, muscle atrophy (assessed by decreased muscle mass at clinical exam and/or assessed by radiographic methods), muscle weakness not attributable to active muscle disease, and muscle dysfunction (e.g., decrease in aerobic exercise tolerance not attributable to other factors).

### Muscle Biopsy

Participants who require a muscle biopsy diagnostic for inclusion in the study will have their historical muscle biopsy slides submitted for biopsy adjudication committee review. Full details of this process are described in the biopsy adjudication charter.

If a participant who requires a muscle biopsy diagnostic does not have historical biopsy data available, the participant will not be considered eligible for enrollment. If a biopsy is performed after a screening failure that is within the course of the participant's routine medical care, then that biopsy may be used if the participant is rescreened.

### C-GTI (Phase 3 Stage Only)

The C-GTI serves as a primary instrument to capture toxicities likely caused by glucocorticoid exposure and enables monitoring of long-term tolerability of glucocorticoids during prolonged use. The C-GTI has 9 functional domains: body mass index, glucose control, BP, lipid metabolism, bone mineral density, muscle strength, skin toxicity, neuropsychiatric effects, and infection. Each domain includes several weighted items that correspond to varying degrees of glucocorticoid toxicity. The bone mineral density domain will not be assessed in this study.

Two analytical scores are generated from the weighted C-GTI items: the CWS and the AIS. The CWS is designed to assess cumulative glucocorticoid toxicity, and the AIS can be used to assess whether a new therapy is effective in reducing glucocorticoid toxicity over time. Together, the CWS and AIS provide complementary information about the ability of an investigational agent to reduce overall glucocorticoid toxicity.

### E. Pharmacokinetics

Blood samples for PK analysis will be collected for measurement of serum concentrations of efgartigimod, as specified in Tables S2 and S3. Sampling must be taken pre-dose, preferably within 2 hours before IMP is administered. During unscheduled visits, blood samples for PK will be collected only if IMP is administered.

Serum efgartigimod concentrations will be determined using a validated assay.

In the phase 2 stage of the study only, an optional additional PK sample will be taken at day 3 (±1 day; opt-in visit) at selected investigative sites until these additional PK samples are obtained from at least 32 participants. This blood sampling should preferably take place at the site, but if needed, the sample may be taken at the participant's home by a qualified person.

Intervention concentration information that may unblind the study will not be reported to investigative sites or blinded personnel.

### F. Pharmacodynamics

Blood samples will be collected for the determination of total IgG serum levels, as described in Tables S2 and S3. Total IgG will be determined using a validated assay.

At screening, total IgG serum levels will be measured to assess eligibility.

For baseline and all post-baseline PD assessment time points, sample collection will be performed pre-dose.

Total IgG concentrations will be quantified at a central laboratory, and results will not be reported to investigative sites or other study personnel to maintain study blind.

PD blood samples collected at the screening visit may be used for methodology validation and/or future research purposes. Such use of these samples is permitted only after obtaining consent from the participant.

### G. Genetics

Assessment will include measuring the IFN signature, which is a normalized score calculated from a set of transcripts that are systemically induced by IFNs (IFN score). This assessment is mandatory, unless it is classified as genetic testing by local regulations. In that case, this assessment is optional and will only be assessed if the participant consents.

### H. Biomarkers

Blood samples will be collected and serum will be aliquoted for the measurement of biomarkers, according to Tables S2 and S3. Full details of measurements and methods are described in a laboratory manual provided separately to sites.

### MSAs and MAAs

Blood samples will be collected and serum will be aliquoted for the measurement of MSAs and MAAs. Samples will be collected according to the schedule described in Tables S2 and S3. For MSAs and MAAs, separate samples will be collected as follows:

A screening sample for presence of specified MSAs used for eligibility and classification of the participant's IIM subtype.

Measurement of MSA/MAA titers. The MSA/MAA panel may include but is not limited to the following: MSA: anti-SRP, -HMGCR, -Mi-2, -TIF1, -SAE, —NXP, -MDA5, -Jo-1, -PL-7, -PL-12, -EJ, -OJ, PM/Scl-75, and Ku; MAA: snRNP, -Ro52 (SSA), -Ro/60 (SSA), -La (SSB).

An optional baseline sample for research in MSA assay testing and methodology. These samples may be used for further MSA assay testing and research in IIM population only. Therefore, where local regulations and IRB/IECs allow, a blood sample may be collected for future exploratory research.

### Other Exploratory Biomarkers

Samples will be collected for the following measurements, as specified in Tables 2 and 3:

Immunophenotyping of PBMC, including but not limited to the assessment of the proportion of CD19+ B cells within the CD45+ population, and may include assessment of myositis-antigen specificity.

Assessment of IFN concentrations, including but not limited to IFN-alpha and IFN-beta protein levels.

Assessment of IFN gene expression score (regulation of IFN-regulated gene expression) by Nanostring technology, including but not limited to the mRNA quantification of the following 6 interferon-stimulated genes (ISGs): interferon alpha inducible protein 27 (IFI27), interferon induced protein 44 (IFI44L), interferon induced protein with tetratricopeptide repeats 1 (IFIT1), ISG15 ubiquitin like modifier (ISG15), radical S-adenosyl methionine domain containing 2 (RSAD2), and sialic acid binding Ig like lectin 1 (SIGLEC1). Also, 3 housekeeping genes will be assessed: actin beta (ACTB), hypoxanthine phosphoribosyltransferase (HPRT1), and RNA polymerase II subunit A (POLR2A). Testing of this sample is mandatory, unless it is classified as a genetic test by local regulations. If it is considered genetic testing, this assessment is optional.

Serological markers of complement activation, including but not limited to C3, C4, and/or split products thereof (C3b/c and C4b/c).

### I. Immunogenicity Assessments

Blood samples will be collected to assess the serum levels of ADA to efgartigimod and plasma levels of antibodies to rHuPH20 from all participants described in Tables S2 and S3. Full details of measurements and methods are described in a laboratory manual provided separately to sites. The immunogenicity sample taken at screening will be used for validation purposes only.

All samples will be analyzed in a 3-tiered approach using validated immunogenicity method. First, all samples will be evaluated in a screening assay (tier 1) and scored positive or negative. Second, screened positive samples will be evaluated in a confirmatory assay (tier 2) to assess the specificity of the immunogenicity response. The samples will be scored confirmed positive or confirmed negative. Samples confirmed positive in tier 2 will be further analyzed in a titration assay to characterize the magnitude of the antibody response and a neutralizing antibody assay to assess the antibodies for neutralizing activity (tier 3).

### J. Objectives and Endpoints

TABLE S4
Objectives and endpoints

| | |
|---|---|
| Objectives | Endpoints |

Primary

| | |
|---|---|
| To evaluate the clinical improvement | Total improvement score (TIS) |
| of efgartigimod PH20 SC treatment | |
| compared with placebo, in addition to | |
| standard-of-care immunomodulatory | |
| therapy | |

Key secondary

| | |
|---|---|
| To evaluate additional measures of | Time to reach TIS ≥20 (first “minimal clinical |
| the efficacy of efgartigimod PH20 SC | improvement”) |
| in achieving clinical response | Percentage of participants with TIS ≥20 |
| | Time to reach TIS ≥40 (first “moderate clinical |
| | improvement”) |
| | Percentage of participants with TIS ≥40 |
| To evaluate the effect of efgartigimod | Change in manual muscle testing-8 (MMT8) score |
| PH20 SC on muscle strength | |
| To evaluate the effect of efgartigimod | Change in Patient Global Assessment of Disease Activity |
| PH20 SC on patient and physician | (PGA) |
| global assessments of disease activity | Change in Physician Global Assessment of Disease Activity |
| | (MDGA) |
| To evaluate the effect of efgartigimod | Change in Health Assessment Questionnaire Disability |
| PH20 SC on physical function | Index (HAQ-DI) |
| parameters | |
| To evaluate the steroid-sparing effect | Change in steroid dose from baseline to last visit |
| of efgartigimod PH20 SC (phase 3 | Proportion of participants achieving target dose of ≤5 mg |
| stage only) | (prednisone equivalent) |
| To evaluate the effect of efgartigimod | Change in the Extramuscular Global Assessment |
| PH20 SC on extramuscular disease | |
| activity | |

Other secondary

| | |
|---|---|
| To evaluate the effect of efgartigimod | Change in abbreviated handheld dynamometry |
| PH20 SC on additional measures of | |
| muscle strength | |
| To evaluate the effect of efgartigimod | Change in Patient Global Impression of Severity (PGI-S) |
| PH20 SC on additional measures of | Patient Global Impression of Change (PGI-C) |
| patient and physician global | Change in Clinical Global Impression of Severity (CGI-S) |
| assessments of disease activity | Clinical Global Impression of Change (CGI-C) |
| To evaluate the effect of efgartigimod | Change in the Myositis Functional Index (FI-3) |
| PH20 SC on muscle endurance | |
| To evaluate the effect of efgartigimod | Change in Patient-Reported Outcomes Information System |
| PH20 SC on pain | (PROMIS) Pain Interference 6a v1.0 |
| | Change in pain numeric rating scale (NRS) assessing worst |
| | pain in the past 7 days |
| To evaluate the effect of efgartigimod | Change in PROMIS Fatigue 7a v1.0 |
| PH20 SC on fatigue | Change in Fatigue NRS assessing worst physical fatigue in |
| | the past 7 days |
| To evaluate the effect of efgartigimod | Change in PROMIS Physical Function 8b v2.0 |
| PH20 SC on additional physical | Change in the Physical Functioning subscale and Physical |
| functioning measures | Component Summary scores of the 36-Item Short Form |
| | Survey version 2 (SF-36v2) |
| To assess the health impact of | Composite Glucocorticoid Toxicity Index (C-GTI) |
| glucocorticoid use and the steroid- | comprising the Aggregate Improvement Score (AIS) and the |
| sparing effect of efgartigimod PH20 | Cumulative Worsening Score (CWS) |
| SC (phase 3 stage only) | |
| To evaluate the effect of efgartigimod | Change in EQ-5D-5L utilities |
| PH20 on health-related quality of life | Change in EQ-5D-5L VAS |
| To evaluate the effect of efgartigimod | Change in the most abnormal enzyme in each participant: |
| PH20 SC on muscle enzymes | creatine kinase (CK), alanine aminotransferase (ALT), |
| | aspartate aminotransaminase (AST), aldolase, or lactate |
| | dehydrogenase (LDH) |
| To evaluate the effect of efgartigimod | Proportion of participants who meet protocol-defined |
| PH20 SC in preventing disease | worsening criteria |
| deterioration | Time to worsening |
| To assess the PK of efgartigimod | Efgartigimod serum concentrations |
| PH20 SC | |
| To assess the PD effect on total IgG | Absolute values, change from baseline, and percent |
| levels of efgartigimod PH20 SC | reduction from baseline in total IgG levels |
| To assess the immunogenicity of | Incidence and prevalence of antidrug antibodies (ADA) |
| efgartigimod and rHuPH20 | against efgartigimod and antibodies against rHuPH20 |
| To assess the safety and tolerability of | Incidence and severity of treatment-emergent AEs (TEAEs), |
| efgartigimod PH20 SC | AESIs, and serious AEs (SAEs) presented by system organ |
| | class (SOC) and preferred term (PT) |
| | Changes and abnormalities in vital signs, electrocardiogram |
| | (ECG), and laboratory parameters |

Exploratory

| | |
|---|---|
| To evaluate the efficacy of | Time to reach TIS ≥60 (first “major clinical improvement”) |
| efgartigimod PH20 SC in achieving | Percentage of participants with TIS ≥60 |
| major clinical response | |
| To evaluate efgartigimod PH20 SC | Percentage of participants withdrawing from IMP because of |
| midstudy treatment failure | lack of clinical response |
| To evaluate the effect of efgartigimod | Change in Myositis Intention-to-Treat Activity Index |
| PH20 SC on additional extramuscular | (MITAX) total and individual scores (constitutional, |
| disease activity parameters | cutaneous, skeletal, gastrointestinal, pulmonary, muscle and |
| | cardiac disease activity) |
| | Change in Myositis Disease Activity Assessment Visual |
| | Analog Scales (MYOACT) total and individual scores |
| | (constitutional, cutaneous, skeletal, gastrointestinal, |
| | pulmonary and cardiac disease activity) |
| To evaluate the effect of efgartigimod | Change in Modified 30-Second Sit-to-Stand Test (m30 STS) |
| PH20 SC on proximal muscle | |
| weakness | |
| To evaluate the effect on physical | Change in the mean daily activity levels over time using step |
| activity using monitor actigraphy | counts, cadence, vector magnitude, and time spent in |
| | different levels of daily activities (mild to moderate to |
| | strenuous activity and sedentary activity variables; average |
| | scores across 7 consecutive days) |
| To assess the efficacy of efgartigimod | TIS in each IIM subtype separately (IMNM, DM, PM) |
| PH20 SC in each IIM subtype | Time to first minimal, moderate, and major improvement on |
| separately (IMNM, DM, PM) | the TIS in each IIM subtype separately (IMNM, DM, PM) |
| | Percentage of participants with at least “minimal clinical |
| | improvement,” percentage of participants with at least |
| | “moderate clinical improvement,” percentage of participants |
| | with “major clinical improvement” in TIS in each IIM |
| | subtype separately (IMNM, DM, PM) |
| | Change in MMT8, FI-3 and CK in participants with IMNM |
| | Change in MMT8, FI-3 and CK in participants with PM |
| | Change in MMT8, most abnormal enzyme, FI-3, Cutaneous |
| | Dermatomyositis Disease Area and Severity Index (CDASI), |
| | Pruritis NRS, and Skindex-16 in participants with DM |
| To assess additional changes in | Change in the remaining 7 subscales and the Mental |
| functional status (beyond physical | Component Summary Score of the SF-36v2 |
| functioning) | |
| To evaluate the effect of efgartigimod | Change in MSA and MAA levels |
| PH20 SC on myositis antibodies | |
| To evaluate the effect of efgartigimod | Changes in lymphocyte dynamics including but not limited |
| PH20 SC on the immunological | to the proportion of CD19+ B cells within the CD45+ |
| profiles | population |
| | Changes in serum cytokine profiles (including but not |
| | limited to interferon [IFN] type I signature) |
| | Changes in serum markers of complement activation |
| | including but not limited to C3, C4, and/or split products |
| | thereof |
| To assess additional measures of the | Duration of clinical response (maximal duration of |
| efficacy of efgartigimod PH20 SC in | consecutive visits with TIS ≥20) |
| achieving clinical response | Duration of moderate clinical response (maximal duration |
| | of consecutive visits with TIS ≥40) |

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

1. A method of treating myositis in a subject in need thereof, the method comprising administering to the subject an effective amount of a human neonatal Fc receptor (FcRn) antagonist, wherein the FcRn antagonist comprises or consists of a variant Fc region or FcRn binding fragment thereof.

2-5. (canceled)

6. The method of claim 1, wherein the FcRn antagonist comprises or consists of a variant Fc region, wherein the variant Fc region comprises or consists of a first Fc domain and a second Fc domain which form a homodimer or heterodimer, and wherein the first Fc domain and/or the second Fc domain comprise amino acids Y, T, E, K, and F at EU positions 252, 254, 256, 433, and 434, respectively.

7. (canceled)

8. The method of claim 6, wherein the first Fc domain and/or the second Fc domain comprise amino acids Y, T, E, K, F, and Y at EU positions 252, 254, 256, 433, 434, and 436, respectively.

9. The method of claim 6, wherein the first Fc domain and/or the second Fc domain comprise an amino acid sequence independently selected from the group consisting of SEQ ID NO: 1, SEQ ID NO: 2, and SEQ ID NO: 3.

10. (canceled)

11. The method of claim 1, wherein the FcRn antagonist is efgartigimod.

12. (canceled)

13. The method of claim 1, wherein the FcRn antagonist is administered subcutaneously to the subject at a fixed dose of 20 mg to 20,000 mg once weekly, once every two weeks, once every three weeks, once every four weeks, once monthly, or once every six weeks: or wherein the FcRn antagonist is administered intravenously at a dose of 0.2 mg/kg to 200 mg/kg once weekly or once every two weeks.

14-16. (canceled)

17. The method of claim 13, wherein the FcRn antagonist is first administered subcutaneously at a fixed dose of about 1000 mg twice on the same day.

18. (canceled)

19. The method of claim 11, wherein the efgartigimod is administered subcutaneously at a fixed dose of about 1000 mg once weekly.

20. The method of claim 11, wherein the efgartigimod is administered subcutaneously at a fixed dose of 1000 mg once weekly.

21. The method of claim 1, wherein the FcRn antagonist is co-formulated with hyaluronidase and administered subcutaneously.

22-26. (canceled)

27. The method of claim 13, wherein the FcRn antagonist is administered intravenously at a dose of 10 mg/kg or 25 mg/kg once weekly or once every two weeks.

28-30. (canceled)

31. The method of claim 1, wherein the FcRn antagonist is administered for at least 24 weeks or for at least 52 weeks.

32. (canceled)

33. The method of claim 1, further comprising administering to the subject a dose of corticosteroid.

34-39. (canceled)

40. The method of claim 1, wherein the myositis is selected from the group consisting of immune-mediated necrotizing myopathy (IMNM), dermatomyositis (DM), juvenile dermatomyositis (JDM), polymyositis (PM), and antisynthetase syndrome (ASyS).

41-44. (canceled)

45. The method of claim 1, wherein the subject has an active DM skin rash.

46-47. (canceled)

48. The method of claim 1, wherein the subject has a detectable serum level of a myositis-associated antibody (MAA) or a myositis-specific antibody (MSA).

49. The method of claim 48, wherein:
the MSA is an anti-aminoacyl-tRNA synthetase antibody, wherein the anti-aminoacyl-tRNA synthetase antibody is selected from the group consisting of anti-Jo-1, anti-PL-7, anti-PL-12, anti-EJ, and anti-OJ antibodies: or
the MSA is selected from the group consisting of anti-SRP, anti-HMGCR, anti-Mi-2, anti-TIF1, anti-SAE, anti-NXP, and anti-MDA5 antibodies: or
the MAA is selected from the group consisting of anti-PM/Scl 75, anti-Ku, anti-snRNP, anti-Ro52 (SSA), anti-Ro/60 (SSA), and anti-La (SSB) antibodies.

50-52. (canceled)

53. The method of claim 48, wherein the subject shows a reduction in serum level of the MAA or MSA following administration of the anti-FcRn antagonist.

54-55. (canceled)

56. The method of claim 1, wherein the subject shows at least a 20 point total improvement score (TIS) after administration of the FcRn antagonist.

57-60. (canceled)

61. The method of claim 1, wherein the subject shows at least a 20% improvement in manual muscle testing-8 (MMT8) score after administration of the FcRn antagonist.

62. (canceled)

63. The method of claim 1, wherein the subject shows at least a 20% decrease in patient's global assessment of disease activity (PGA) after administration of the FcRn antagonist.

64. (canceled)

65. The method of claim 1, further comprising measuring at least one muscle-associated enzyme in the subject, wherein a reduction in the at least one muscle-associated enzyme in the subject is indicative of disease improvement.

66-70. (canceled)

71. A method of treating myositis in a subject that has received a first FcRn antagonist and is receiving a corticosteroid dosing regimen, the method comprising:
a) administering to the subject an effective amount of a second FcRn antagonist;
b) measuring in vitro a serum level of an MAA or an MSA in a blood sample taken from the subject; and
c) comparing the serum level of the MAA or the MSA to a reference value associated with myositis in the subject,
wherein the corticosteroid dosing regimen is maintained if the serum level of the MAA or the MSA in the sample is greater than or equal to the reference value, or wherein the corticosteroid dosing regimen is tapered if the serum level of the MAA or the MSA is less than the reference value.

72. A method for treating myositis in a subject comprising:
(a) administering to the subject one or more initial doses of an effective amount of a first FcRn antagonist,
(b) administering to the subject one or more further doses of an effective amount of a second FcRn antagonist if the serum level of an MAA or an MSA in the subject after step (a) is greater than or equal to a reference value associated with myositis in the subject, or discontinuing treatment with the first FcRn antagonist if the serum level of the MAA or the MSA in the subject after step (a) is less than a reference value associated with active disease in the subject.

73. A method for treating myositis in a subject, the method comprising: administering to the subject an effective amount of a second FcRn antagonist,
wherein the myositis has relapsed in the subject following prior therapy with a first FcRn antagonist and wherein the subject has a serum level of an MAA or an MSA that is greater than or equal to a reference value associated with myositis in the subject.

74-102. (canceled)
